(19) United States
(12) Patent Application Publication
(10) Pub. No.: US 2011/0046042 A1
(43) Pub. Date: Feb. 24, 2011

(54) Long Interspersed Nuclear Element Polypeptide Compositions and Methods of Use Thereof

(75) Inventors: Douglas F. Nixon, San Francisco, CA (US); Keith Garrison, Alameda, CA (US); Duncan Meiklejohn, West Lebanon, NH (US); Mario Ostrowski, Toronto (CA); R. Bradley Jones, Toronto (CA); Ashish Agrawal, Fremont, CA (US); Frederick M. Hecht, San Francisco, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignees: THE J. DAVID GLADSTONE INSTITUTES, San Francisco, CA (US); THE REGENTS OF THE UNIVERSITY OF CALIFORNIA, Oakland, CA (US)

(21) Appl. No.: 12/677,278

(22) PCT Filed: Sep. 19, 2008
(86) PCT No.: PCT/US2008/010883

§ 371 Date: Nov. 5, 2010

Related U.S. Application Data
(60) Provisional application No. 60/973,993, filed on Sep. 20, 2007.

(51) Int. Cl. A61K 38/08; C07K 7/06; C07H 21/04; C07K 7/08; A61P 31/18; C12N 15/63; A61K 31/7088; A61K 38/10; C12Q 1/02; A61P 35/00; A61P 31/12

(52) U.S. Cl. 514/3.7; 514/21.6; 530/328; 536/23.1; 530/327; 435/320.1; 514/44 R; 514/3.8; 514/21.5; 435/29

(57) ABSTRACT

The present invention provides LINE polypeptides; and compositions, including immunogenic compositions, comprising a subject LINE polypeptide. The present invention provides a recombinant nucleic acid comprising a nucleotide sequence encoding a subject LINE polypeptide. A subject composition is useful for stimulating a T-cell immune response to a LINE peptide. The present invention further provides methods of stimulating an immune response in an individual to a retrovirus- or lentivirus-infected cell. The present invention further provides methods of treating cancers that are associated with tissues in which LINE polypeptides are aberrantly expressed. Also provided are methods of treating disorders, involving decreasing an immune response to a LINE polypeptide.

### CROSS-REFERENCE

This application claims the benefit of U.S. Provisional Patent Application No. 60/973,993, filed Sep. 20, 2007, which application is incorporated herein by reference in its entirety.

### STATEMENT REGARDING FEDERALLY SPONSORED RESEARCH

This invention was made with Government support under Federal Grant Nos. AI68498 and AI41531 awarded by the National Institutes of Health. The Government has certain rights in this invention.

### BACKGROUND

Retroelements can be divided into at least three classes: exogenous retroviruses, retrotransposons containing long terminal repeats (LTRs) and retrotransposons lacking LTRs. The non-LTR retrotransposons can be subdivided further into long interspersed nuclear elements (LINES) and short interspersed nuclear elements (SINEs). LINE-1 (“L1”) is a non-LTR retrotransposon found in all mammalian genomes and is the most common human LINE. There are ˜500,000 LINE-1 elements in the human genome which account for ˜17% of its total sequence. Out of the total LINE-1 genomic population, there are ˜100 full length elements, the remainder being truncated to varying extents. LINE-1 is transcribed to give rise to a ˜6 kb mRNA that encodes two proteins, ORF1p (or p40) and ORF2p (or p150). ORF1p encodes an RNA binding protein with nucleic acid chaperone activity, and ORF2p encodes the enzymes necessary for retrotransposition: endonuclease and reverse transcriptase. LINE-1 retrotransposition occurs by a target-primed reverse transcription (TPRT) mechanism, where reverse transcription occurs in concert with integration.

### Literature

Bogerd et al. (2006) Proc. Natl. Acad. Sci. USA 103(23):8780-5; Hohjoh et al. (1997) EMBO J. 16 (19):6034-6043; Boissinot et al. (2000) Mol. Biol. Evol. 18(12):2186-2194; Ergun et al. (2004)J. Biol. Chem. 279 (26): 27753-27763; U.S. Pat. No. 5,280,108.

### SUMMARY OF THE INVENTION

The present invention provides LINE polypeptides; and compositions, including immunogenic compositions, comprising a subject LINE polypeptide. The present invention provides a recombinant nucleic acid comprising a nucleotide sequence encoding a subject LINE polypeptide. A subject composition is useful for stimulating a T-cell immune response to a LINE peptide, such that a LINE peptide present on a virus-infected cell is recognized by a LINE-specific T cell. The present invention further provides methods of stimulating an immune response in an individual to a retrovirus- or lentivirus-infected cell. The present invention further provides methods of treating cancers that are associated with tissues in which LINE polypeptides are aberrantly expressed. Also provided are methods of treating disorders, involving decreasing an immune response to a LINE polypeptide.

### FEATURES OF THE INVENTION

The present invention provides an immunogenic composition comprising a LINE polypeptide. In some embodiments, a subject immunogenic composition comprises a subject LINE polypeptide and a pharmaceutically acceptable carrier. A subject LINE polypeptide can comprise an amino acid sequence having at least about 75% amino acid sequence identity to any one of SEQ ID NOs:1-22. In further embodiments, a subject LINE polypeptide comprises an amino acid sequence set forth in any one of SEQ ID NOs:1-101. A subject immunogenic composition can be formulated in various ways, including for parenteral administration or for administration to a mucosal tissue. In some embodiments, a subject immunogenic composition further comprises an adjuvant. In some embodiments, the adjuvant is aluminum hydroxide, MF59, or monophosphoryl lipidA.

The present invention provides an immunogenic composition comprising a nucleic acid comprising a nucleotide sequence encoding a LINE polypeptide. The encoded LINE polypeptide can comprise an amino acid sequence having at least about 75% amino acid sequence identity to any one of SEQ ID NOs:1-22. In some embodiments, a subject immunogenic composition is formulated for parenteral administration or for administration to a mucosal tissue. The nucleic acid comprising a nucleotide sequence encoding a LINE polypeptide will in some embodiments be a recombinant vector, e.g., a viral vector.

A method of inducing a T lymphocyte response in an individual to a host cell infected with or at risk of infection with a pathogenic virus is also provided, wherein the method comprises administering to an individual in need thereof an immunogenic composition comprising a subject LINE polypeptide and a pharmaceutically acceptable carrier or a nucleic acid comprising a nucleotide sequence encoding a subject LINE polypeptide. In some embodiments, the T lymphocyte response comprises a CD8⁺ T cell response or a CD4⁺ T cell response. In further embodiments, the T lymphocyte response comprises a mucosal T lymphocyte response. Individuals who are suitable for treatment using a subject method include, e.g., individuals infected with, or at risk of becoming infected with, a pathogenic virus such as a human retrovirus, e.g., a human immunodeficiency virus.

A subject method for inducing a T lymphocyte response in an individual to a host cell infected with or at risk of infection with a pathogenic virus can be carried out on an individual who has not been infected with a pathogenic virus. In these embodiments, a subject method can involve administering an immunogenic composition comprising a LINE polypeptide and a pharmaceutically acceptable carrier or a nucleic acid comprising a nucleotide sequence encoding a LINE polypeptide to an individual who has not been infected with a pathogenic virus. In other embodiments, a subject method induces a T lymphocyte response in an individual who has been infected with the pathogenic virus.

Also provided are methods of inducing a T lymphocyte response to a cancer cell in an individual, where the cancer cell exhibits LINE expression and displays LINE epitopes on its surface, the methods generally involving administering to the individual a subject immunogenic composition comprising a subject LINE polypeptide and a pharmaceutically acceptable carrier, or administering to the individual a subject immunogenic composition comprising a nucleic acid comprising a nucleotide sequence encoding a subject LINE polypeptide.

Also provided are methods of generating a population of CD8⁺ T cells specific for a LINE polypeptide, wherein the methods generally involve contacting a population of unstimulated CD8⁺ T cells in vitro with a subject isolated LINE polypeptide in association with an antigen-presenting platform, wherein the contacting provides for production of a population of LINE peptide-specific CD8⁺ T cells.

### BRIEF DESCRIPTION OF THE DRAWINGS

FIGS. 1A and 1B depict expression of L1-P150 in HIV-1-infected primary CD4⁺ T cells.

FIG. 2 depicts immune responses to L1 in peripheral blood of HIV-1-infected individuals, but not in uninfected individuals.

FIGS. 3A-E depict specific recognition of HIV-1-infected cells by L1-specific CD8⁺ T cells.

FIGS. 4A and 4B depict elimination of HIV-1-infected cells and suppression of virus production by L1-specific CD8⁺ T cell clones.

FIGS. 5A-E depict an L1-specific T cell clone recognition assay with diverse HIV-1 panel and HIV-2 (experiment “TO1”).

FIGS. 6A-D depict L1-specific T cell clone diverse HIV-1 panel recognition assay “SF1.”

FIG. 7 depicts L1-specific T cell clone diverse HIV-1 panel recognition assay “TO2.”

FIG. 8 depicts correlation of the degree of responsiveness of clone L1-3O to autologous cells infected with diverse HIV-1 isolates with the level of infection.

FIG. 9 depicts blockade of infected cell recognition by pre-incubation with anti-HLA-A,B,C antibody.

FIG. 10 is a table which provides candidate LINE polypeptides identified using BLAST searches of LINE amino acid sequences against HIV-1 proteins.

FIG. 11 is a table which provides exemplary sequence alignments between subject LINE polypeptides and HIV protein sequences resulting from BLAST search of LINE amino acid sequences against HIV-1 proteins.

FIG. 12 is a table which presents LINE-1 peptides identified using in silico epitope prediction.

FIG. 13 is a table which provides ELISPOT assay results for isolated LINE polypeptides LiD9R, LiE 13E, LiK10I, Lil9C, Lim12T, LiN13V and LiQ9E.

FIG. 14 is a table which provides ELISPOT assay results for isolated LINE polypeptides LiIV9, LiKI9, LiRV9, LiTV9.

FIG. 15 is a table which presents peptide sequences and characteristics of the peptides referred to in FIG. 2.

FIG. 16 is a table which provides information regarding HIV-1-infected subjects.

FIG. 17 is a table which provides information regarding HIV-1-infected subjects.

FIG. 18 is a table which provides information regarding viral isolates.

FIG. 19 depicts the results of a LINE-1 (L1)-specific T cell clone killing assay with diverse HIV-1 panel.

FIG. 20 depicts amino acid sequences of 15-mer LINE polypeptides.

FIGS. 21A-C depict amino acid sequences of 15-mer LINE polypeptides.

### DEFINITIONS

As used herein, a “biological sample” encompasses a variety of sample types obtained from an individual and can be used in a diagnostic or monitoring assay. The term “biological sample” encompasses blood and other liquid samples of biological origin, solid tissue samples such as a biopsy specimen or tissue cultures or cells derived therefrom and the progeny thereof. The term “biological sample” also includes samples that have been manipulated in any way after their procurement, such as by treatment with reagents; washed; or enrichment for certain cell populations, such as CD4⁺ T lymphocytes, CD8+ T lymphocytes, glial cells, macrophages, tumor cells, peripheral blood mononuclear cells (PBMC), and the like. The term “biological sample” encompasses a clinical sample, and also includes cells in culture, cell supernatants, tissue samples, organs, bone marrow, blood, plasma, serum, cerebrospinal fluid, and the like.

The term “retrovirus” is well understood in the art, and includes single-stranded, positive sense, enveloped RNA viruses that include, e.g., the genus Gammaretrovirus (e.g., murine mammary tumor virus); the genus Epsilonretrovirus; the genus Alpharetrovirus (e.g., avian leukosis virus); the genus Betaretrovirus; the genus Deltaretrovirus (e.g., bovine leukemia virus; human T-lymphotrophic virus (HTLV)); the genus Lentivirus; and the genus Spumavirus. The term “lentivirus,” as used herein, refers to a genus of viruses of the Retroviridae family, and includes human immunodeficiency virus-1 (HIV-1); human immunodeficiency virus-2 (HIV-2); simian immunodeficiency virus (SIV); and feline immunodeficiency virus (FIV).

As used herein, “gene delivery vehicle” refers to a construct which is capable of delivering, and, within some embodiments expressing, one or more gene(s) or nucleotide sequence(s) of interest in a host cell. Representative examples of such vehicles include viral vectors, nucleic acid expression vectors, naked DNA, and certain eukaryotic cells (e.g., producer cells).

As used herein, “operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct-the expression thereof. Thus, for example, intervening untranslated yet transcribed sequences can be present between a promoter sequence and the coding sequence and the promoter sequence can still be considered “operably linked” to the coding sequence.

The terms “polypeptide,” “peptide,” and “protein,” used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like. NH₂ refers to the free amino group present at the amino terminus of a polypeptide. COOH refers to the free carboxyl group present at the carboxyl terminus of a polypeptide. In keeping with standard polypeptide nomenclature, J. Biol. Chem., 243 (1969), 3552-59 is used.

As used herein the term “isolated” is meant to describe a polynucleotide, a polypeptide, or a cell that is in an environment different from that in which the polynucleotide, the polypeptide, or the cell naturally occurs. An isolated genetically modified host cell may be present in a mixed population of genetically modified host cells. An isolated polypeptide will in some embodiments be synthetic. “Synthetic polypeptides” are assembled from amino acids, and are chemically synthesized in vitro, e.g., cell-free chemical synthesis, using procedures known to those skilled in the art. An isolated polypeptide will in some embodiments be purified.

By “purified” is meant a compound of interest (e.g., a polypeptide) has been separated from components that accompany it in nature. “Purified” can also be used to refer to a compound of interest (e.g., a polypeptide) separated from components that can accompany it during manufacture (e.g., in chemical synthesis). In some embodiments, a compound (e.g., a polypeptide) is substantially pure when it is at least 50% to 60%, by weight, free from organic molecules with which it is naturally associated or with which it is associated during manufacture. In some embodiments, the preparation is at least 75%, at least 90%, at least 95%, or at least 99%, by weight, of the compound of interest. Thus, e.g., a subject polypeptide that is “purified” is present in a composition where the polypeptide is present in an amount of at least 75%, at least 90%, at least 95%, or at least 99%, by weight, of the composition. A substantially pure compound can be obtained, for example, by extraction from a natural source (e.g., bacteria), by chemically synthesizing a compound, or by a combination of purification and chemical modification. A substantially pure compound can also be obtained by, for example, enriching a sample having a compound that binds an antibody of interest. Purity can be measured by any appropriate method, e.g., chromatography, mass spectroscopy, high performance liquid chromatography analysis, etc.

The term “heterologous,” as used herein in the context of a LINE polypeptide, where a LINE polypeptide fusion protein comprises a LINE polypeptide and a “heterologous” polypeptide, refers to a polypeptide that is other than a LINE polypeptide, e.g., a polypeptide that is not normally associated in nature with a LINE polypeptide. For example, a heterologous polypeptide bears no significant amino acid sequence identity to the LINE polypeptide, e.g., the heterologous polypeptide has less than about 50%, less than about 40%, less than about 30%, or less than about 20% amino acid sequence identity to the LINE polypeptide.

An “antigen” is defined herein to include any substance that may be specifically bound by an antibody molecule or a T cell receptor. An “immunogen” is an antigen that is capable of initiating lymphocyte activation resulting in an antigen-specific immune response.

By “epitope” is meant a site on an antigen to which specific B cells and/or T cells respond. The term is also used interchangeably with “antigenic determinant” or “antigenic determinant site.” B cell epitope sites on proteins, polysaccharides, or other biopolymers may be composed of moieties from different parts of the macromolecule that have been brought together by folding. Epitopes of this kind are referred to as conformational or discontinuous epitopes, since the site is composed of segments of the polymer that are discontinuous in the linear sequence but are continuous in the folded conformation(s). Epitopes that are composed of single segments of biopolymers or other molecules are termed continuous or linear epitopes. T cell epitopes are generally linear peptides. Antibodies that recognize the same epitope can be identified in a simple immunoassay showing the ability of one antibody to block the binding of another antibody to a target antigen.

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

“Recombinant,” as used herein in the context of a nucleic acid, means that a particular nucleic acid (DNA or RNA) is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Thus, e.g., the term “recombinant” polynucleotide or nucleic acid refers to one which is not naturally occurring, e.g., is made by the artificial combination of two otherwise separated segments of sequence through human intervention.

“Operably linked” refers to a juxtaposition wherein the components so described are in a relationship permitting them to function in their intended manner. For instance, a promoter is operably linked to a coding sequence if the promoter affects its transcription or expression.

The terms “cancer,” “neoplasm,” and “tumor” are used interchangeably herein to refer to cells which exhibit relatively autonomous growth, so that they exhibit an aberrant growth phenotype characterized by a significant loss of control of cell proliferation. Cells of interest for treatment in the present application include precancerous, malignant, pre-metastatic, metastatic, and non-metastatic cells, as well as carcinoma in situ.

“Cancerous phenotype” generally refers to any of a variety of biological phenomena that are characteristic of a cancerous cell, which phenomena can vary with the type of cancer. The cancerous phenotype is generally identified by abnormalities in, for example, cell growth or proliferation (e.g., uncontrolled growth or proliferation), regulation of the cell cycle, cell mobility, cell-cell interaction, or metastasis, etc.

The terms “subject,” “individual,” “host,” and “patient” are used interchangeably herein to refer to a mammal, including, but not limited to, murines (rats, mice), felines, non-human primates (e.g., simians), humans, canines, ungulates, etc.

The terms “treatment,” “treating,” “treat,” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a long interspersed nuclear element (LINE) polypeptide” includes a plurality of such polypeptides and reference to “the immunogenic composition” includes reference to one or more immunogenic compositions and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present invention provides LINE polypeptides (e.g., isolated LINE polypeptides; synthetic LINE polypeptides); and compositions, including immunogenic compositions, comprising a subject LINE polypeptide. The present invention provides a nucleic acid comprising a nucleotide sequence encoding a subject LINE polypeptide. The present invention provides compositions comprising a subject LINE polypeptide or a subject LINE polynucleotide. The immunogenic compositions are useful for stimulating a T cell immune response to a LINE polypeptide, e.g., on a virus-infected cell. For example, a subject immunogenic composition is useful for stimulating a T cell immune response that can recognize a LINE polypeptide (or fragment thereof) on an HTLV- or an HIV-infected cell. As described in more detail below, stimulating a T cell immune response to a LINE polypeptide or fragment thereof on an HTLV- or an HIV-infected cell can provide treatment of a viral infection (e.g., an HTLV infection, an HIV infection). The present invention further provides methods of stimulating an immune response in an individual to a retrovirus- or lentivirus-infected cell. The present invention further provides methods of treating cancers in which LINE polypeptides are aberrantly expressed by cancerous cells. For example, a subject immunogenic composition is useful for stimulating a T cell immune response that can recognize a LINE polypeptide (or fragment thereof) on a cancerous or precancerous cell. Also provided are methods of treating disorders, involving decreasing an immune response to a LINE polypeptide.

In some embodiments, a subject immunogenic composition induces a T cell immune response specific for a retrovirus-infected cell, e.g., a human immunodeficiency virus (HIV)-infected cell. Epitopes displayed by a subject LINE polypeptide stimulate or enhance a T cell immune response to the epitopes. Where the LINE epitopes are also present on the surface of a retrovirus-infected cell, a T cell response to the retrovirus-infected cell also occurs. A “T cell immune response” includes one or more of: 1) an increase in the number and/or activity of CD4⁺ T cells specific for the LINE epitope; 2) an increase in the number and/or activity (e.g., cytotoxicity) of CD8⁺ T cells specific for the LINE epitope; and 3) secretion of cytokines that induce or are indicative of a Th1-type immune response. Cytokines that induce or are indicative of a Th1 immune response include, but are not limited to, interferon-gamma (IFN-γ) and IL-2. T cell immune responses that are stimulated with a subject immunogenic composition include a mucosal T cell immune response and a systemic T cell immune response.

A subject immunogenic composition can be formulated in any of a variety of ways, including a formulation suitable for intravenous administration, subcutaneous administration, or other parenteral route of administration; a formulation suitable for administration to a mucosal tissue; and the like. The present invention provides pharmaceutical formulations comprising a subject immunogenic composition.

The present invention further provides LINE polypeptide compositions that are suitable for use in monitoring a patient's response to treatment for a retrovirus infection (e.g., an HTLV infection). Thus, the present invention further provides methods for monitoring a patient's response to treatment for a retrovirus infection (e.g., an HTLV infection).

The present invention further provides LINE polypeptide compositions that are suitable for use in monitoring a patient's response to treatment for a lentivirus infection (e.g., an HIV infection). Thus, the present invention further provides methods for monitoring a patient's response to treatment for a lentivirus infection (e.g., an HIV infection).

### Isolated LINE Polypeptides

The present invention provides LINE polypeptides, and compositions comprising a subject LINE polypeptide. A subject LINE polypeptide finds use in, e.g., generating immunogenic compositions (e.g., for enhancing an immune response in an individual to a LINE polypeptide or enhancing an immune response in an individual to an HIV epitope or polypeptide); generating immunomodulatory compositions (e.g., for reducing an immune response in an individual to a LINE polypeptide; monitoring patient response to therapy, e.g., therapy for a retrovirus infection; staging a disease; detecting a disease; and for generating CD8⁺ T cells for adoptive transfer methods. In some embodiments, a subject LINE polypeptide is isolated. In some embodiments, a subject isolated LINE polypeptide is synthetic (e.g., chemically synthesized). Thus, the present invention provides synthetic LINE polypeptides. In the discussion that follows, the term “subject isolated LINE polypeptide,” or simply “subject LINE polypeptide,” is used; however, it should be understood that the following discussion applies equally to a “subject synthetic LINE polypeptide.”

### LINE Polypeptides

LINE polypeptides include polypeptides encoded by any LINE clade, family, sub-family, class or group, e.g., CRE, R2, R4, L1, L2, RTE, Tad1, R1, LOA, Jockey, CR1, and I, and any subgroup thereof. LINE clades, families, sub-families, classes, groups, and subgroups are known in the art. See, e.g., Malik et al, Molecular Biology and Evolution 16 (6): 793. (1999); Lovsin et al, Molecular Biology and Evolution 18: 2213-2224.

In some embodiments, a subject isolated LINE polypeptide (or a subject synthetic LINE polypeptide) comprises a polypeptide comprising from about 6, 7, 8, 9, 10, 11, 12, 13-15, 15-17, 17-20, from 20 to 25, from 25 to 50, from 50 to 75, from 75 to 100, from 100 to 150, from 150 to 200, from 200 to 250, from 250 to 300, from 300 to 350, or from 350 to 400, or more, contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence of a LINE-encoded polypeptide. LINE-encoded polypeptides include polypeptides encoded by the ORF1p (p40) and ORF2p (p150) of a LINE.

In other embodiments, a subject isolated LINE polypeptide comprises a polypeptide comprising from about 6, 7, 8, 9, 10, 11, 12, 13-15, 15-17, 17-20, from 20 to 25, from 25 to 50, from 50 to 75, from 75 to 100, from 100 to 150, from 150 to 200, from 200 to 250, from 250 to 300, from 300 to 350, or from 350 to 400, or more, contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence of a LINE-encoded polypeptide, provided that the LINE-encoded polypeptide has an amino acid sequence which is shorter than that of the polypeptides encoded by either ORF1p (p40) or ORF2p (p150) of LINE-1.

In some embodiments, a subject isolated LINE polypeptide comprises a stretch of from about 6, 7, 8, 9, 10, 11, 12, 13-15, 15-17, 17-20, or from 20 to 25, or more contiguous amino acids having at least about 50%, at least about 60%, at least about 70%, at least about 80%, at least about 85%, at least about 90%, at least about 95%, at least about 98%, at least about 99%, or 100% amino acid sequence identity to a stretch of amino acids of the same length in an HIV-encoded protein, polypeptide, or epitope.

In other embodiments, a subject isolated LINE polypeptide consists of or consists essentially of a stretch of from about 6, 7, 8, 9, 10, 11, 12, 13-15, 15-17, 17-20, or from 20 to 25, or more contiguous amino acids having at least about 50%, at least about 60%, at least about 70%, at least about 75%, at least about 80%, at least about 85%, at least about 90%, at least about 95%, at least about 98%, at least about 99%, or 100% amino acid sequence identity to a stretch of amino acids of the same length in an HIV-encoded protein, polypeptide, or epitope.

In further embodiments, a subject isolated LINE polypeptide is a LINE polypeptide which is identified and isolated, wherein the LINE polypeptide is identified by searching a retroviral protein database, e.g., an HIV protein database for short nearly exact matches with a LINE polypeptide sequence. Such a search may be conducted, for example, by utilizing a BLAST search (Altschul et al., 1997) of LINE amino acid sequences against HIV proteins using the short nearly exact match (e-value=200000, PAM30 matrix, SEG filter OFF, word size=2) parameters for the algorithm. The short, nearly exact match parameters for the BLAST algorithm facilitate the detection of short matches between peptide sequences, normally overwhelmed by the divergence of the proteins. Although these regions of similarity or identity may not have significance for phylogenetic analysis, they can represent highly conserved functional domains of proteins and/or regions of potential cross-reactivity for T-cell recognition.

A subject LINE polypeptide can also be identified by analyzing the protein sequences predicted from LINE-1 ORF1 and ORF2 using epitope prediction software. For example, epitope peptides from LINE-1 can be identified by analyzing the protein sequences predicted from LINE-1ORF1 and ORF2 with the NETCTL™ epitope prediction program. NETCTL™ analyzes an entire protein to identify the sites of proteosomal cleavage, the subset of resulting breakdown products with the best potential to bind the transporter associated with antigen processing (TAP) machinery, and the peptides within those breakdown products with the best binding affinity for different human leukocyte antigen (HLA) molecules (Larsen et al., European Journal of Immunology. 35(8): 2295-303. 2005).

A subject isolated LINE polypeptide can be from 6 amino acids in length up to the length of a naturally-occurring LINE polypeptide, e.g., a LINE polypeptide can be 6 amino acids (aa), 7 aa, 8 aa, 9 aa, 10 aa, 11 aa, 12-15 aa, 15-20 aa, 20-25 aa, 25-30 aa, 30-40 aa, 40-50 aa, 50-100 aa, or longer than 100 amino acids, e.g., 100 aa to 150 aa, 150 aa to 200 aa. In some embodiments, a subject isolated LINE polypeptide has a length of from about 6 aa to about 150 aa, from about 6 aa to about 10 aa, from about 10 aa to about 15 aa, from about 15 aa to about 20 aa, from about 20 aa to about 25 aa, from about 25 aa to about 30 aa, from about 30 aa to about 40 aa, from about 40 aa to about 50 aa, from about 50 aa to about 75 aa, from about 75 aa to about 100 aa, from about 100 aa to about 125 aa, or from about 125 aa to about 150 aa.

Exemplary, non-limiting examples of LINE-encoded polypeptides are found in GenBank Accession Nos. AAC51261 (SEQ ID NO:23), AAC51262 (SEQ ID NO:24), AAC51263 (SEQ ID NO:25), AAC51264 (SEQ ID NO:26), AAC51265 (SEQ ID NO:27), AAC51266 (SEQ ID NO:28), AAC51267 (SEQ ID NO:29), AAC51268 (SEQ ID NO:30), AAC51269 (SEQ ID NO:31), AAC51270 (SEQ ID NO:32), AAC51271 (SEQ ID NO:33), AAC51272 (SEQ ID NO:34), AAC51273 (SEQ ID NO:35), AAC51274 (SEQ ID NO:36), AAC51275 (SEQ ID NO:37), AAC51276 (SEQ ID NO:38), AAC51277 (SEQ ID NO:39), AAC51278 (SEQ ID NO:40), AAC51279 (SEQ ID NO:41), etc.

In some embodiments, a subject isolated LINE polypeptide comprises a polypeptide comprising about 6, 7, 8, 9, 10, 11, or 12 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO:1:

| | |
|---|---|
| MNEMKREGKFRE. | (SEQ ID NO: 1) |

In some embodiments, a subject LINE polypeptide comprises a polypeptide comprising about 6, 7, 8, 9 or 10 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO: 2:

| | |
|---|---|
| SQLKELEKQE. | (SEQ ID NO: 2) |

In some embodiments, a subject isolated LINE polypeptide comprises a polypeptide comprising about 6, 7, 8, 9, 10, 11 or 12 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO:3:

| | |
|---|---|
| MLRAAREKGWVT. | (SEQ ID NO: 3) |

For example, a subject LINE polypeptide can comprise the amino acid sequence MLRAAREKGRVT; or the amino acid sequence MLRAAREEGRVT.

In some embodiments, a subject isolated LINE polypeptide comprises a polypeptide comprising about 6, 7, 8, 9 or 10 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO:4:

| | |
|---|---|
| KIDRLLARLI. | (SEQ ID NO: 4) |

For example, a subject LINE polypeptide can comprise the amino acid sequence KIDRPLARLI; or the amino acid sequence KIDRPLSRLI.

In some embodiments, a subject isolated LINE polypeptide comprises a polypeptide comprising about 6, 7, 8 or 9 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO:5:

| | |
|---|---|
| LRAAREKGC. | (SEQ ID NO: 5) |

In some embodiments, a subject isolated LINE polypeptide comprises a polypeptide comprising about 6, 7, 8, 9, 10, 11, 12 or 13 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO:6:

| | |
|---|---|
| NGKQKKAGFAILV. | (SEQ ID NO: 6) |

For example, a subject LINE polypeptide can comprise the amino acid sequence NGKQKKAGVAILV.

In some embodiments, a subject isolated LINE polypeptide comprises a polypeptide comprising about 6, 7, 8 or 9 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO:7:

| | |
|---|---|
| DELREEGVR. | (SEQ ID NO: 7) |

In some embodiments, a subject isolated LINE polypeptide comprises a polypeptide comprising about 6, 7, 8 or 9 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO:8:

| | |
|---|---|
| TMRYHLTPV. | (SEQ ID NO: 8) |

In some embodiments, a subject isolated LINE polypeptide comprises a polypeptide comprising about 6, 7, 8 or 9 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO:9:

| | |
|---|---|
| RPNLRLIGV. | (SEQ ID NO: 9) |

For example, a subject LINE polypeptide can comprise the amino acid sequence RPNLHLIGV.

In some embodiments, a subject isolated LINE polypeptide comprises a polypeptide comprising about 6, 7, 8 or 9 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO:10:

| | |
|---|---|
| KVIYRFNAI. | (SEQ ID NO: 10) |

For example, a subject LINE polypeptide can comprise the amino acid sequence KVIYRFSAI; or KVTYRFNTI.

In some embodiments, a subject isolated LINE polypeptide comprises a polypeptide comprising about 6, 7, 8 or 9 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO:11:

| | |
|---|---|
| IVYLENPIV. | (SEQ ID NO: 11) |

For example, a subject LINE polypeptide can comprise the amino acid sequence IVYLENPMV; or the amino acid sequence IVCLKNPIV.

In some embodiments, a subject isolated LINE polypeptide comprises a polypeptide comprising about 6, 7, 8, or 9 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO:12:

| | |
|---|---|
| SLQEIWDYV. | (SEQ ID NO: 12) |

In some embodiments, a subject isolated LINE polypeptide comprises a polypeptide comprising about 6, 7, 8, or 9 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO:13:

| | |
|---|---|
| NLEECITRI. | (SEQ ID NO: 13) |

In some embodiments, a subject isolated LINE polypeptide comprises a polypeptide comprising about 6, 7, 8, or 9 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO:14:

| | |
|---|---|
| TPRHIIVRF. | (SEQ ID NO: 14) |

For example, a subject LINE polypeptide can comprise the amino acid sequence TPRHVIVRF; or the amino acid sequence TPRHILVRF; or the amino acid sequence TPRHILVKF.

In some embodiments, a subject isolated LINE polypeptide comprises a polypeptide comprising about 6, 7, 8, or 9 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO:15:

| | |
|---|---|
| LLFNIVLEV. | (SEQ ID NO: 15) |

In some embodiments, a subject isolated LINE polypeptide comprises a polypeptide comprising about 6, 7, 8, or 9 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO:16:

| | |
|---|---|
| YTMEYYAAI. | (SEQ ID NO: 16) |

In some embodiments, a subject isolated LINE polypeptide comprises a polypeptide comprising about 6, 7, 8, or 9 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO:17:

| | |
|---|---|
| RARIAKSIL. | (SEQ ID NO: 17) |

For example, a subject LINE polypeptide can comprise the amino acid sequence RARMAKSIL; or the amino acid sequence RACIAKSIL; or the amino acid sequence RAHIAKSTL.

In some embodiments, a subject isolated LINE polypeptide comprises a polypeptide comprising about 6, 7, 8, or 9 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO:18:

| | |
|---|---|
| APRFIKQVL. | (SEQ ID NO: 18) |

In some embodiments, a subject isolated LINE polypeptide comprises a polypeptide comprising about 6, 7, 8, or 9 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO:19:

| | |
|---|---|
| ISYPAKLSF. | (SEQ ID NO: 19) |

For example, a subject LINE polypeptide can comprise the amino acid sequence ISFPAKLSF; or the amino acid sequence ISYPATLGF.

In some embodiments, a subject isolated LINE polypeptide comprises a polypeptide comprising about 6, 7, 8, or 9 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO:20:

| | |
|---|---|
| SSPATEQSW. | (SEQ ID NO: 20) |

For example, a subject LINE polypeptide can comprise the amino acid sequence SSPATDQSW; or the amino acid sequence SSLATEQSW.

In some embodiments, a subject isolated LINE polypeptide comprises a polypeptide comprising about 6, 7, 8, or 9 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO:21:

| | |
|---|---|
| KATVTKTAW. | (SEQ ID NO: 21) |

For example, a subject LINE polypeptide can comprise the amino acid sequence KATVTKTVW; or the amino acid sequence KATVTKTAC.

In some embodiments, a subject isolated LINE polypeptide comprises a polypeptide comprising about 6, 7, 8, or 9 contiguous amino acids of an amino acid sequence having at least about 50%, at least about 60%, at least about 70%, at least about 75%, at least about 80%, at least about 85%, at least about 90%, at least about 95%, at least about 98%, at least about 99%, or 100% amino acid sequence identity to the amino acid sequence set forth in SEQ ID NO:22:

| | |
|---|---|
| RVNRQPTTW. | (SEQ ID NO: 22) |

For example, a subject LINE polypeptide can comprise the amino acid sequence RANRQPTTW; or the amino acid sequence RVNRQPTEW; or the amino acid sequence RVNRQATEW.

In some embodiments, a subject isolated LINE polypeptide comprises one or more of the following amino acid sequences:

| | |
|---|---|
| MNEMKREGKFRE; | (SEQ ID NO: 1) |
| SQLKELEKQE; | (SEQ ID NO: 2) |
| MLRAAREKGWVT; | (SEQ ID NO: 3) |
| KIDRLLARLI; | (SEQ ID NO: 4) |
| LRAAREKGC; | (SEQ ID NO: 5) |
| NGKQKKAGFAILV; | (SEQ ID NO: 6) |
| DELREEGVR; | (SEQ ID NO: 7) |
| TMRYHLTPV; | (SEQ ID NO: 8) |
| RPNLRLIGV; | (SEQ ID NO: 9) |
| KVIYRFNAI; | (SEQ ID NO: 10) |
| IVYLENPIV; | (SEQ ID NO: 11) |
| SLQEIWDYV; | (SEQ ID NO: 12) |
| NLEECITRI; | (SEQ ID NO: 13) |
| TPRHIIVRF; | (SEQ ID NO: 14) |
| LLFNIVLEV; | (SEQ ID NO: 15) |
| YTMEYYAAI; | (SEQ ID NO: 16) |
| RARIAKSIL; | (SEQ ID NO: 17) |
| APRFIKQVL; | (SEQ ID NO: 18) |
| ISYPAKLSF; | (SEQ ID NO: 19) |
| SSPATEQSW; | (SEQ ID NO: 20) |
| KATVTKTAW; | (SEQ ID NO: 21) |
| RVNRQPTTW; | (SEQ ID NO: 22) |
| MLRAAREKGRVT; | (SEQ ID NO: 77) |
| MLRAAREEGRVT; | (SEQ ID NO: 78) |
| KIDRPLARLI; | (SEQ ID NO: 79) |
| KIDRPLSRLI; | (SEQ ID NO: 80) |
| NGKQKKAGVAILV; | (SEQ ID NO: 81) |
| RPNLHLIGV; | (SEQ ID NO: 82) |
| KVIYRFSAI; | (SEQ ID NO: 83) |
| KVTYRFNTI; | (SEQ ID NO: 84) |
| IVYLENPMV; | (SEQ ID NO: 85) |
| IVCLKNPIV; | (SEQ ID NO: 86) |
| TPRHVIVRF; | (SEQ ID NO: 87) |
| TPRHILVRF; | (SEQ ID NO: 88) |
| TPRHILVKF; | (SEQ ID NO: 89) |
| RARMAKSIL; | (SEQ ID NO: 90) |
| RACIAKSIL; | (SEQ ID NO: 91) |
| RAHIAKSTL; | (SEQ ID NO: 92) |
| ISFPAKLSF; | (SEQ ID NO: 93) |
| ISYPATLGF; | (SEQ ID NO: 94) |
| SSPATDQSW; | (SEQ ID NO: 95) |
| SSLATEQSW; | (SEQ ID NO: 96) |
| KATVTKTVW; | (SEQ ID NO: 97) |
| KATVTKTAC; | (SEQ ID NO: 98) |
| RANRQPTTW; | (SEQ ID NO: 99) |
| RVNRQPTEW; | (SEQ ID NO: 100) |
| and | |
| RVNRQATEW. | (SEQ ID NO: 101) |

In any of the above-described embodiments, a subject LINE polypeptide can be from 6 amino acids in length up to the length of a naturally-occurring LINE polypeptide, e.g., a LINE polypeptide can be 6 amino acids (aa), 7 aa, 8 aa, 9 aa, 10 aa, 11 aa, 12-15 aa, 15-20 aa, 20-25 aa, 25-30 aa, 30-40 aa, 40-50 aa, 50-100 aa, or longer than 100 amino acids, e.g., 100 aa to 150 aa, 150 aa to 200 aa. In any of the above-described embodiments, a subject LINE polypeptide has a length of from about 6 aa to about 150 aa, e.g., from about 6 aa to about 10 aa, from about 10 aa to about 15 aa, from about 15 aa to about 20 aa, from about 20 aa to about 25 aa, from about 25 aa to about 30 aa, from about 30 aa to about 40 aa, from about 40 aa to about 50 aa, from about 50 aa to about 75 aa, from about 75 aa to about 100 aa, from about 100 aa to about 125 aa, or from about 125 aa to about 150 aa.

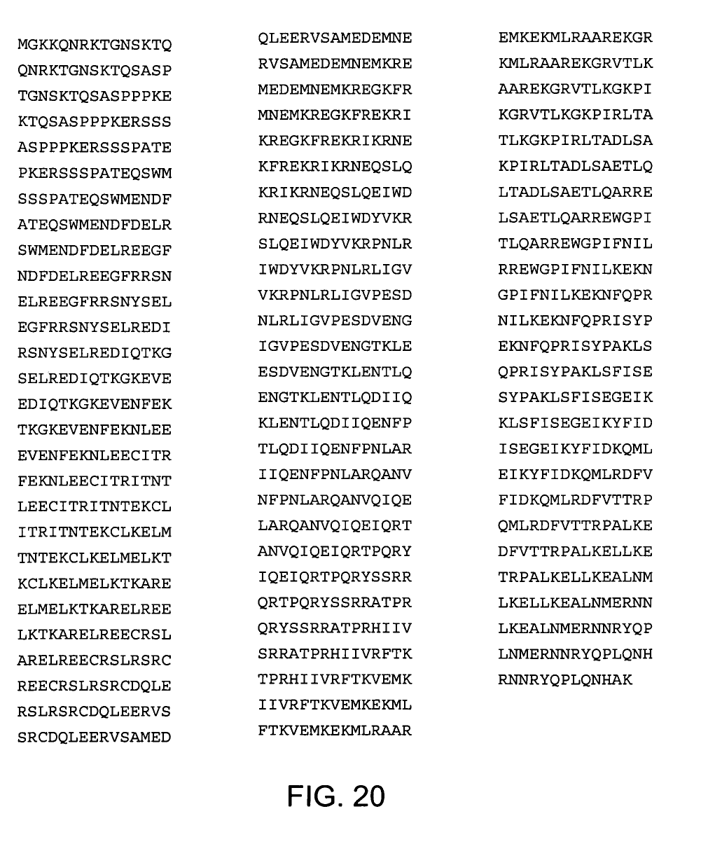

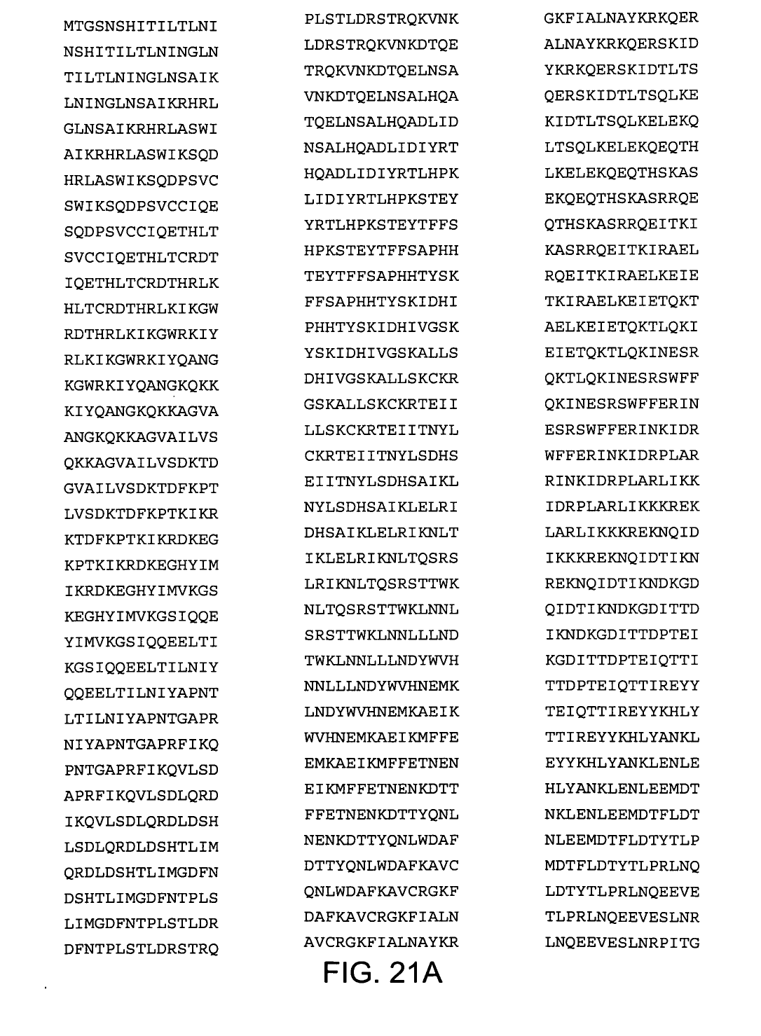

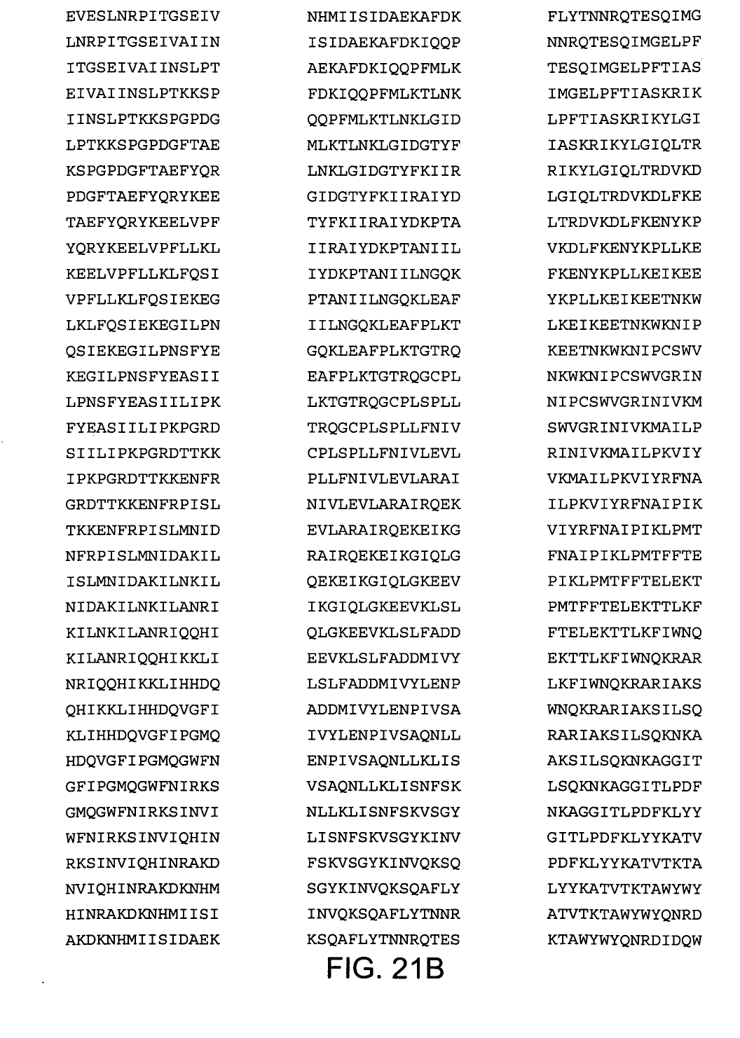

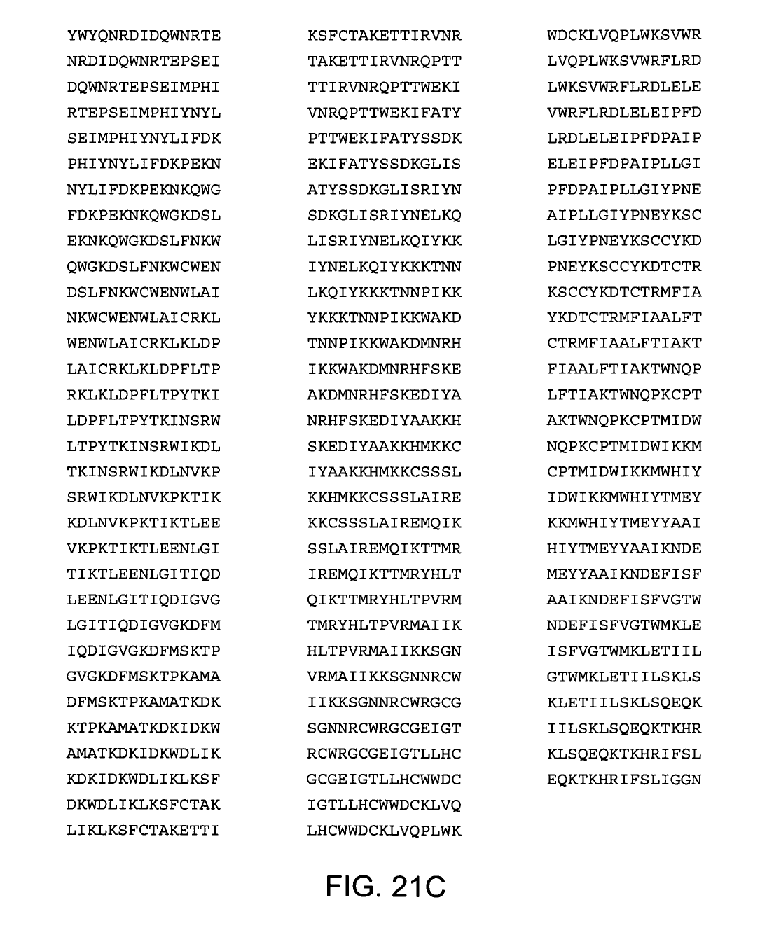

In some embodiments, a subject isolated LINE polypeptide comprises one or more of the 15-mer amino acid sequences depicted in FIG. 20, and in FIGS. 21A-C.

In some embodiments, a LINE polypeptide is a fusion protein, e.g., a LINE fusion protein comprises a LINE polypeptide covalently linked to a heterologous protein, where the heterologous protein is also referred to as a “fusion partner.” In some embodiments, the fusion partner is attached to the N-terminus of the LINE protein, e.g., NH₂-fusion partner-LINE-COOH. In other embodiments, the fusion partner is attached to the C-terminus of the LINE protein, e.g., NH₂-LINE-fusion partner-COOH. In other embodiments, the fusion partner is internal to the LINE protein, e.g., NH₂-(LINE₁-FP-(LINE₂-COOH)₂, where FP is a fusion partner, and LINE₁ and LINE₂ are N-terminal and C-terminal regions, respectively, of LINE.

Suitable fusion partners include, but are not limited to, immunological tags such as epitope tags, including, but not limited to, hemagglutinin, FLAG, myc, and the like; proteins that provide for a detectable signal, including, but not limited to, fluorescent proteins, enzymes (e.g., (β-galactosidase, luciferase, horse radish peroxidase, alkaline phosphatase, etc.), and the like; polypeptides that facilitate purification or isolation of the fusion protein, e.g., metal ion binding polypeptides such as 6His tags, glutathione-S-transferase, and the like; polypeptides that provide for subcellular localization; and polypeptides that provide for secretion from a cell. Fusion partners that provide for a detectable signal are also referred to as “reporters.” In some embodiments, a fusion partner is an immunomodulatory polypeptide other than a LINE polypeptide, e.g., an antigen, a cytokine, etc.

### Multimerized LINE Polypeptides

In some embodiments, a subject isolated LINE polypeptide is multimerized, e.g., two or more LINE polypeptides are linked in tandem. Multimers include dimers, trimers, tetramers, pentamers, etc. Monomeric LINE polypeptides are linked to one another directly or via a linker. Thus, in some embodiments, a subject LINE polypeptide has the formula (X₁-(Y)₀₋₄₀-X₂-(Y)₀₋₄₀)ₙ, where X₁ and X₂ are LINE polypeptides, Y is a linker, and n is an integer from 1 to about 10 (e.g., n=1, 2, 3, 4, 5, 6, 7, 8, 9, or 10). Where a linker is used, Y is one or more amino acids, or other linking groups. X₁ and X₂ can be the same or different, e.g., can have the same amino acid sequence, or can differ from one another in amino acid sequence. Thus, e.g., a subject LINE polypeptide can have the formula X₁-(Y)₀₋₄₀-X₂, e.g., where the LINE polypeptide is a dimer. As one non-limiting example, where a subject LINE polypeptide has the formula (X₁-(Y)₀₋₄₀-X₂-(Y)₀₋₄₀)ₙ, X₁ can be MNEMKREGKFRE (SEQ ID NO:1), and X₂ can be SQLKELEKQE (SEQ ID NO:2). As another non-limiting example, where a subject LINE polypeptide has the formula (X₁-(Y)₀₋₄₀-X₂-(Y)₀₋₄₀)ₙ, X₁ and X₂ can both be MNEMKREGKFRE (SEQ ID NO:1).

As another example, a subject LINE polypeptide can have the formula X₁-(Y)₀₋₄₀-X₂-(Y)₀₋₄₀-X₃, e.g., where the LINE polypeptide is a trimer. As one non-limiting example, where a subject LINE polypeptide has the formula X₁-(Y)₀₋₄₀-X₂-(Y)₀₋₄₀-X₃, X₁ can be MNEMKREGKFRE (SEQ ID NO:1), X₂ can be SQLKELEKQE (SEQ ID NO:2), and X₃ can be MLRAAREKGWVT (SEQ ID NO:3). As one non-limiting example, where a subject LINE polypeptide has the formula X₁-(Y)₀₋₄₀-X₂-(Y)₀₋₄₀-X₃, X₁, X₂, and X₃ can all be MNEMKREGKFRE (SEQ ID NO:1).

Where Y is a spacer peptide, it is generally of a flexible nature, although other chemical linkages are not excluded. Currently, it is contemplated that the most useful linker sequences will generally be peptides of between about 2 and about 40 amino acids in length, e.g., from about 2 amino acids to about 10 amino acids, from about 10 amino acids to about 20 amino acids, or from about 6 amino acids to about 25 amino acids in length. These linkers are generally produced by using synthetic, linker-encoding oligonucleotides to couple the proteins. Peptide linkers with a degree of flexibility will generally be used. The linking peptides may have virtually any amino acid sequence, bearing in mind that the preferred linkers will have a sequence that results in a generally flexible peptide. The use of small amino acids, such as glycine and alanine, are of use in creating a flexible peptide. Exemplary peptide linkers include (Gly)₂₋₄₀ (SEQ ID NO:74), (Ser)₂₋₄₀ (SEQ ID NO:75), and (Ala)₂₋₄₀ (SEQ ID NO:76). The creation of such sequences is routine to those of skill in the art. Many different linkers are commercially available and are considered suitable for use according to the disclosed embodiments. However, any flexible linker generally between about 2 amino acids and about 40 amino acids, e.g., from about 6 amino acids to about 10 amino acids in length may be used. Linkers may have virtually any sequence that results in a generally flexible peptide.

Linkages for homo- or hetero-polymers or for coupling to carriers can be provided in a variety of ways. For example, cysteine residues can be added at both the amino- and carboxyl-termini, where the peptides are covalently bonded via controlled oxidation of the cysteine residues. Also useful are a large number of heterobifunctional agents which generate a disulfide link at one functional group end and a peptide link at the other, including N-succidimidyl-3-(2-pyridyldithio) proprionate (SPDP). This reagent creates a disulfide linkage between itself and a cysteine residue in one protein and an amide linkage through the amino on a lysine or other free amino group in the other. A variety of such disulfide/amide forming agents is known. See, for example, Immun. Rev, 62:185 (1982). Other bifunctional coupling agents form a thioether rather than a disulfide linkage. Many of these thioether forming agents are commercially available and include reactive esters of 6-maleimidocaproic acid, 2 bromoacetic acid, 2-iodoacetic acid, 4-(N-maleimido-methyl)cyclohexane-1-carboxylic acid and the like. The carboxyl groups can be activated by combining them with succinimide or 1-hydroxy-2-nitro-4-sulfonic acid, sodium salt. An exemplary coupling agent is succinimidyl 4-(N-maleimidomethyl) cyclohexane-1-carboxylate (SMCC). Of course, it will be understood that linkage should not substantially interfere with either of the linked groups to function for its intended use, e.g., as an immunogen.

### Carriers

In some embodiments, a subject isolated LINE polypeptide is linked to a carrier. The term “linked,” as used herein interchangeably with the term “coupled,” refers to proximately associated, e.g., the LINE polypeptide and the carrier are in close spatial proximity. In some embodiments, the linkage is a covalent linkage. In other embodiments, the linkage is a non-covalent linkage. In some embodiments, the LINE polypeptide is linked directly to the carrier. In other embodiments, the LINE polypeptide is linked indirectly, e.g., via a linker molecule.

Examples of suitable carriers include large, slowly metabolized macromolecules such as: proteins; polysaccharides, such as sepharose, agarose, cellulose, cellulose beads and the like; polymeric amino acids such as polyglutamic acid, polylysine, and the like; amino acid copolymers; inactivated virus particles; inactivated bacterial toxins such as toxoid from diphtheria, tetanus, cholera, leukotoxin molecules; liposomes; inactivated bacteria; dendritic cells; and the like. Carriers are described in further detail below.

Suitable carriers are well known in the art, and include, e.g., thyroglobulin, albumins such as human serum albumin, tetanus toxoid; Diphtheria toxoid; polyamino acids such as poly(D-lysine:D-glutamic acid); VP6 polypeptides of rotaviruses; influenza virus hemagglutinin, influenza virus nucleoprotein; hepatitis B virus core protein, hepatitis B virus surface antigen; purified protein derivative (PPD) of tuberculin from Mycobacterium tuberculosis; inactivated Pseudomonas aeruginosa exotoxin A (toxin A); Keyhole Limpet Hemocyanin (KLH); filamentous hemagglutinin (FHA) of Bordetella pertussis; T helper cell (Th) epitopes of tetanus toxoid (TT) and Bacillus Calmette-Guerin (BCG) cell wall; recombinant 10 kDa, 19 kDa and 30-32 kDa proteins from M. leprae or from M. tuberculosis, or any combination of these proteins; and the like. See, e.g., U.S. Pat. No. 6,447,778 for a discussion of carriers, and for methods of conjugating peptides to carriers.

Pseudomonas aeruginosa exotoxin A (toxin A) has been used effectively as a carrier in conjugate vaccines. Pseudomonas aeruginosa exotoxin A may be purified from the supernatant of fermentor-grown cultures of Pseudomonas aeruginosa PA 103. Toxin A has been classified as a superantigen based upon results in animals. Toxin A can be completely and irreversibly detoxified by covalent coupling to adipic acid dihydrazide (ADH), a 4 carbon spacer molecule. This step destroys the ADPR-transferase activity of the toxin molecule, hence rendering it nontoxic. The non-reacted hydrazide group can be used to covalently couple a polypeptide to toxin A. Toxin A may also be coupled to a polypeptide using a carbodiimide reagent.

PPD-peptide conjugates are conveniently prepared with glutaraldehyde as coupling agent. See, e.g., Rubinstein et al. (1995) AIDS 9:243-51.

The methods by which a subject polypeptide is conjugated with a carrier include disulfide linkages through a C terminal peptide cysteine linkage, coupling with glutaraldehyde solution for two hours, coupling with tyrosine, or coupling with water soluble carbodiimide.

In some embodiments, a subject isolated LINE polypeptide is lipidated. Lipidation increases a cytotoxic T cell (CTL) response to the peptide that is linked to the lipid. The lipid residue, such as palmitic acid or the like, is attached to the amino terminus of the peptide. The lipid can be attached directly to the peptide, or, indirectly via a linkage, such as a Ser-Ser, Gly, Gly-Gly, Ser linkage or the like. As another example, E. coli lipoprotein, such as tripalmitoyl-S-glycerylcysteinyl-seryl-serine (P₃ CSS), can be used to prime specific CTL when covalently attached to the peptide. See, Deres et al., Nature 342:561-564 (1989). A LINE polypeptide can be conjugated with uncharged fatty acid residues of different chain lengths and degrees of unsaturation, ranging from acetic to stearic acid as well as to negatively charged succinyl residues via the appropriate carboxylic acid anhydrides. See, e.g., U.S. Pat. No. 6,419,931.

A subject isolated LINE polypeptide may be conjugated directly or indirectly, e.g., via a linker molecule, to a carrier. A wide variety of linker molecules are known in the art and can be used in the conjugates. The linkage from the peptide to the carrier may be through a peptide reactive side chain, or the N- or C-terminus of the peptide. A linker may be an organic, inorganic, or semi-organic molecule, and may be a polymer of an organic molecule, an inorganic molecule, or a co-polymer comprising both inorganic and organic molecules.

If present, the linker molecules are generally of sufficient length to permit the LINE polypeptide and a linked carrier to allow some flexible movement between the LINE polypeptide and the carrier. The linker molecules are generally about 6-50 atoms long. The linker molecules may also be, for example, aryl acetylene, ethylene glycol oligomers containing 2-10 monomer units, diamines, diacids, amino acids, or combinations thereof. Other linker molecules which can bind to polypeptides may be used in light of this disclosure.

### Compositions

The present invention provides compositions comprising a subject isolated LINE polypeptide. Compositions comprising a subject isolated LINE polypeptide can include one or more of: a salt, e.g., NaCl, MgCl, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a protease inhibitor; and the like. In some embodiments, as described in more detail below, a subject LINE composition is an immunogenic composition. In other embodiments, as described in more detail below, a subject LINE composition is a pharmaceutical composition, e.g., a composition comprising a subject isolated LINE polypeptide and a pharmaceutically acceptable excipient.

In some embodiments, a subject composition comprises a single type (or “species”) of a subject LINE polypeptide, e.g., in some embodiments, the LINE polypeptides in a subject composition all comprise substantially the same amino acid sequence. In other embodiments, a subject immunogenic composition comprises two or more different LINE polypeptides, e.g., the composition comprises a population of subject LINE polypeptides, the members of which population can differ in amino acid sequence. A subject composition can comprise from two to about 20 different LINE polypeptides, e.g., a subject composition can comprise two, three, four, five, six, seven, eight, nine, ten, 11-15, or 15-20 different LINE polypeptides, each having an amino acid that differs from the amino acid sequences of the other LINE polypeptides. For example, in some embodiments, a subject composition comprises a first LINE polypeptide having a first amino acid sequence; and at least a second LINE polypeptide having a second amino acid sequence, where the second amino acid sequence differs from the first amino acid sequence. As another example, in some embodiments, a subject composition comprises a first LINE polypeptide having a first amino acid sequence; second LINE polypeptide having a second amino acid sequence, where the second amino acid sequence differs from the first amino acid sequence; and at least a third LINE polypeptide having a third amino acid sequence, where the third amino acid sequence differs from both the first and the second amino acid sequences. In other embodiments, a subject composition comprises a multimerized LINE polypeptide, as described above.

### Production of LINE Polypeptides

A subject LINE polypeptide can be produced in a number of ways, including, e.g., by chemical synthesis, where the LINE polypeptide is a “synthetic” polypeptide; by isolation and purification from a naturally-occurring source; and by recombinant means, where the LINE polypeptide is a “recombinant” polypeptide. Recombinant means for producing a subject LINE polypeptide are well known in the art, and involve genetically modifying a host cell with a polynucleotide comprising a nucleotide sequence encoding a subject LINE polypeptide, culturing the host cell in vitro under conditions and for a suitable time such that the LINE polypeptide is produced by the genetically modified cell, and isolating the LINE polypeptide produced by the genetically modified cell.

### Pharmaceutical Compositions

The present invention provides a pharmaceutical composition comprising a subject LINE polypeptide, the composition comprising a subject LINE polypeptide and a pharmaceutically acceptable excipient.

A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents.

A subject LINE polypeptide pharmaceutical composition can be prepared by dissolving, suspending or emulsifying a subject LINE polypeptide in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

### Immunogenic Compositions Comprising a Subject LINE Polypeptide

The present invention provides immunogenic compositions comprising a subject LINE polypeptide. LINE polypeptides and isolated LINE polypeptides suitable for inclusion in a subject immunogenic composition are as described above.

In some embodiments, a subject immunogenic composition comprises a LINE polypeptide that comprises one or more T cell epitopes that, when presented on the surface of a retrovirus-infected cell, induce a T cell immune response specific for a retrovirus-infected cell, e.g., a human immunodeficiency virus (HIV)-infected cell or an HTLV-infected cell. A “T cell immune response” includes one or more of: 1) an increase in the number and/or activity of CD4⁺ T cells specific for the LINE epitope; 2) an increase in the number and/or activity of CD8⁺ T cells specific for the LINE epitope; and 3) secretion of cytokines that induce or are indicative of a Th1-type immune response. Cytokines that induce or are indicative of a Th1 immune response include, but are not limited to, interferon-gamma (IFN-γ) and IL-2.

In certain embodiments, administration of a subject immunogenic composition results in T cell mediated killing of a retrovirus-infected cell, e.g. an HIV infected cell, via specific T cell recognition of a LINE polypeptide or fragment thereof on the surface of a retrovirus-infected cell. In other embodiments, administration of a subject immunogenic composition results in T cell mediated killing of a retrovirus-infected cell, e.g. an HIV infected cell, via cross-reactivity of a T cell specific for a LINE polypeptide or fragment thereof with a retroviral epitope presented on the surface of a lentivirus-infected cell.

A subject immunogenic composition comprising a subject LINE polypeptide can be formulated in a number of ways, as described in more detail below. In some embodiments, a subject immunogenic composition comprises single species of LINE polypeptide, e.g., the immunogenic composition comprises a population of LINE polypeptides, substantially all of which have the same amino acid sequence. In other embodiments, a subject immunogenic composition comprises two or more different LINE polypeptides, e.g., the immunogenic composition comprises a population of LINE polypeptides, the members of which population can differ in amino acid sequence. A subject immunogenic composition can comprise from two to about 20 different LINE polypeptides, e.g., a subject immunogenic composition can comprise two, three, four, five, six, seven, eight, nine, ten, 11-15, or 15-20 different LINE polypeptides, each having an amino acid that differs from the amino acid sequences of the other LINE polypeptides. For example, in some embodiments, a subject immunogenic composition comprises a first LINE polypeptide having a first amino acid sequence; and at least a second LINE polypeptide having a second amino acid sequence, where the second amino acid sequence differs from the first amino acid sequence. As another example, in some embodiments, a subject immunogenic composition comprises a first LINE polypeptide having a first amino acid sequence; second LINE polypeptide having a second amino acid sequence, where the second amino acid sequence differs from the first amino acid sequence; and at least a third LINE polypeptide having a third amino acid sequence, where the third amino acid sequence differs from both the first and the second amino acid sequences. In other embodiments, a subject immunogenic composition comprises a multimerized LINE polypeptide, as described above.

A subject immunogenic composition can provided in a pharmaceutically acceptable diluent such as an aqueous solution, e.g., a saline solution, a semi-solid form (e.g., gel), or in powder form. Such diluents can be inert.

### Adjuvants

In some embodiments, a subject immunogenic composition comprises a subject LINE polypeptide (isolated or synthetic), and an adjuvant. Suitable adjuvants include those suitable for use in humans. Examples of known suitable adjuvants that can be used in humans include, but are not necessarily limited to, alum, aluminum phosphate, aluminum hydroxide, MF59 (4.3% w/v squalene, 0.5% w/v polysorbate 80 (Tween 80), 0.5% w/v sorbitan trioleate (Span 85)), a CpG-containing nucleic acid (where the cytosine is unmethylated), QS21 (saponin adjuvant), MPL (Monophosphoryl Lipid A), 3DMPL (3-O-deacylated MPL), extracts from Aquilla, ISCOMS (see, e.g., Sjölander et al. (1998) J. Leukocyte Biol. 64:713), LT/CT mutants, poly(D,L-lactide-co-glycolide) (PLG) microparticles, Quil A, interleukins, and the like. For veterinary applications including but not limited to animal experimentation, one can use Freund's, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion.

Further exemplary adjuvants to enhance effectiveness of the composition include, but are not limited to: (1) oil-in-water emulsion formulations (with or without other specific immunostimulating agents such as muramyl peptides (see below) or bacterial cell wall components), such as for example (a) MF59™ (WO90/14837; Chapter 10 in Vaccine design: the subunit and adjuvant approach, eds. Powell & Newman, Plenum Press 1995), containing 5% Squalene, 0.5% Tween 80 (polyoxyethylene sorbitan mono-oleate), and 0.5% Span 85 (sorbitan trioleate) (optionally containing muramyl tri-peptide covalently linked to dipalmitoyl phosphatidylethanolamine (MTP-PE)) formulated into submicron particles using a microfluidizer, (b) SAF, containing 10% Squalane, 0.4% Tween 80, 5% pluronic-blocked polymer L121, and thr-MDP either microfluidized into a submicron emulsion or vortexed to generate a larger particle size emulsion, and (c) RIBI™ adjuvant system (RAS), (Ribi Immunochem, Hamilton, Mont.) containing 2% Squalene, 0.2% Tween 80, and one or more bacterial cell wall components such as monophosphorylipid A (MPL), trehalose dimycolate (TDM), and cell wall skeleton (CWS), e.g., MPL+CWS (DETOX™); (2) saponin adjuvants, such as QS21 or STIMULON™ (Cambridge Bioscience, Worcester, Mass.) may be used or particles generated therefrom such as ISCOMs (immunostimulating complexes), which ISCOMS may be devoid of additional detergent e.g. WO 00/07621; (3) Complete Freund's Adjuvant (CFA) and Incomplete Freund's Adjuvant (IFA); (4) cytokines, such as interleukins (e.g. IL-1, IL-2, IL-4, IL-5, IL-6, IL-7, IL-12 (WO99/44636), etc.), interferons (e.g. gamma interferon), macrophage colony stimulating factor (M-CSF), tumor necrosis factor (TNF), other TNF superfamily molecules (e.g., CH40L, OX40L, and the like), etc.; (5) monophosphoryl lipid A (MPL) or 3-O-deacylated MPL (3dMPL) e.g. GB-2220221, EP-A-0689454, optionally in the substantial absence of alum when used with pneumococcal saccharides e.g. WO00/56358; (6) combinations of 3dMPL with, for example, QS21 and/or oil-in-water emulsions e.g. EP-A-0835318, EP-A-0735898, EP-A-0761231; (7) oligonucleotides comprising CpG motifs [Krieg Vaccine 2000, 19, 618-622; Krieg Curr Opin Mol Ther2001 3:15-24; Roman et al., Nat. Med., 1997, 3, 849-854; Weiner et al., PNAS USA, 1997, 94, 10833-10837; Davis et al, J. Immunol., 1998, 160, 870-876; Chu et al., J. Exp. Med, 1997, 186, 1623-1631; Lipford et al, Eur. J. Immunol., 1997, 27, 2340-2344; Moldoveanu et al., Vaccine, 1988, 16, 1216-1224, Krieg et al., Nature, 1995, 374, 546-549; Klinman et al., PNAS USA, 1996, 93, 2879-2883; Ballas et al, J. Immunol, 1996, 157, 1840-1845; Cowdery et al, J. Immunol, 1996, 156, 4570-4575; Halpern et al, Cell Immunol., 1996, 167, 72-78; Yamamoto et al, Jpn. J. Cancer Res., 1988, 79, 866-873; Stacey et al, J. Immunol., 1996, 157, 2116-2122; Messina et al, J. Immunol, 1991, 147, 1759-1764; Yi et al, J. Immunol, 1996, 157, 4918-4925; Yi et al, J. Immunol, 1996, 157, 5394-5402; Yi et al, J. Immunol, 1998, 160, 4755-4761; and Yi et al, J. Immunol, 1998, 160, 5898-5906; International patent applications WO96/02555, WO98/16247, WO98/18810, WO98/40100, WO98/55495, WO98/37919 and WO98/52581]i.e. containing at least one CG dinucleotide, where the cytosine is unmethylated; (8) a polyoxyethylene ether or a polyoxyethylene ester e.g. WO99/52549; (9) a polyoxyethylene sorbitan ester surfactant in combination with an octoxynol (WO01/21207) or a polyoxyethylene alkyl ether or ester surfactant in combination with at least one additional non-ionic surfactant such as an octoxynol (WO01/21152); (10) a saponin and an immunostimulatory oligonucleotide (e.g. a CpG oligonucleotide) (WO00/62800); (11) an immunostimulant and a particle of metal salt e.g. WO00/23105; (12) a saponin and an oil-in-water emulsion e.g. WO99/11241; (13) a saponin (e.g. QS21)+3dMPL+IM2 (optionally+a sterol) e.g. WO98/57659; (14) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE), etc.

A subject immunogenic composition can include a conventional pharmaceutically acceptable excipient, such as pharmaceutical grades of mannitol, lactose, starch, magnesium stearate, sodium saccharin, talcum, cellulose, glucose, sucrose, magnesium, carbonate, and the like. A subject immunogenic composition can include one or more pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antigen (e.g., a subject LINE polypeptide) in these formulations can vary widely, and can be selected based on various factors such as fluid volumes, viscosities, body weight and the like in accordance with the particular mode of administration selected and the patient's needs. The resulting compositions may be in the form of a solution, suspension, tablet, pill, capsule, powder, gel, cream, lotion, ointment, aerosol or the like.

The protein concentration of a subject immunogenic composition in the pharmaceutical formulations can vary widely, e.g., less than about 0.1%, from about 0.1% to about 2%, from about 2% to 20%, or from about 20% to about 50%, or more, by weight, and will be selected on the basis of various factors such as fluid volumes, viscosities, etc., in accordance with the particular mode of administration selected.

In some embodiments, a subject LINE polypeptide is formulated with one or more lipids. For example, liposomes of various sizes can be made. Small liposomes or vesicles formed are unilamellar and have a size in the range of about 20 to 400 nanometers and can be produced by subjecting multi-lamellar vesicles to ultrasound, by extrusion under pressure through membranes having pores of defined size, or by high pressure homogenization. Larger unilamellar liposomes having a size in the range of about 0.1 to 1 μm in diameter can be obtained when the lipid is solubilized in an organic solvent or a detergent and the solubilized agent is removed by evaporation or dialysis, respectively. The fusion of smaller unilamellar liposomes by methods requiring particular lipids or stringent dehydration-hydration conditions can yield unilamellar vessels as large or larger than cells.

Liposomes can comprise one or more cationic lipids, e.g., DDAB, dimethyldioctadecyl ammonium bromide; N-[1-(2,3-Dioloyloxy)propyl]-N,N,N-trimethylammonium methylsulfate; 1,2-diacyl-3-trimethylammonium-propanes, (including but not limited to, dioleoyl (DOTAP), dimyristoyl, dipalmitoyl, disearoyl); 1,2-diacyl-3-dimethylammonium-propanes, (including but not limited to, dioleoyl, dimyristoyl, dipalmitoyl, disearoyl) DOTMA, N-[1-[2,3-bis(oleoyloxy)]propyl]-N,N,N-trimethylammonium chloride; DOGS, dioctadecylamidoglycylspermine; DC-cholesterol, 3β-[N-(N′,N′-dimethylaminoethane)carbamoyl]cholesterol; DOSPA, 2,3-dioleoyloxy-N-(2(sperminecarboxamido)-ethyl)-N,N-dimethyl-1-propanaminium trifluoroacetate; 1,2-diacyl-sn-glycero-3-ethylphosphocholines (including but not limited to dioleoyl (DOEPC), dilauroyl, dimyristoyl, dipalmitoyl, distearoyl, palmitoyl-oleoyl); β-alanyl cholesterol; CTAB, cetyl trimethyl ammonium bromide; diC14-amidine, N-t-butyl-N′-tetradecyl-3-tetradecylaminopropionamidine; 14Dea2, O,O′-ditetradecanolyl-N-(trimethylammonioacetyl)diethanolamine chloride; DOSPER, 1,3-dioleoyloxy-2-(6-carboxy-spermyl)-propylamide; N,N,N,N′-tetramethyl-N,N′-bis(2-hydroxylethyl)-2,3-dioleoyloxy-1,4-butanediammonium iodide; 1-[2-acyloxy)ethyl]2-alkyl(alkenyl)-3-(2-hydroxyethyl)imidazolinium chloride derivatives such as 1-[2-(9(Z)-octadecenoyloxy)ethyl]-2-(8(Z)-heptadecenyl-3-(2-hydroxyethyl)imidazolinium chloride (DOTIM), 1-[2-(hexadecanoyloxy)ethyl]-2-pentadecyl-3-(2-hydroxyethyl)imidazolinium chloride (DPTIM); 1-[2-tetradecanoyloxy)ethyl]-2-tridecyl-3-(2-hydroxyethyl)imidazolium chloride (DMTIM)—as described in Solodin et al. (1995) Biochem. 43:13537-13544; 2,3-dialkyloxypropyl quaternary ammonium compound derivates, containing a hydroxyalkyl moiety on the quaternary amine, such as 1,2-dioleoyl-3-dimethyl-hydroxyethyl ammonium bromide (DORI); 1,2-dioleyloxypropyl-3-dimethyl-hydroxyethyl ammonium bromide (DORIE); 1,2-dioleyloxypropyl-3-dimethyl-hydroxypropyl ammonium bromide (DORIE-HP); 1,2-dioleyloxypropyl-3-dimethyl-hydroxybutyl ammonium bromide (DORIE-HB); 1,2-dioleyloxypropyl-3-dimethyl-hydroxypentyl ammonium bromide (DORIE-HPe); 1,2-dimyristyloxypropyl-3-dimethyl-hydroxylethyl ammonium bromide (DMRIE); 1,2-dipalmityloxypropyl-3-dimethyl-hydroxyethyl ammonium bromide (DPRIE); 1,2-disteryloxypropyl-3-dimethyl-hydroxyethyl ammonium bromide (DSRIE)—as described, e.g., in Felgner et al. (1994)J. Biol. Chem. 269:2550-2561. Many of the above-mentioned lipids are available commercially from, e.g., Avanti Polar Lipids, Inc.; Sigma Chemical Co.; Molecular Probes, Inc.; Northerm Lipids, Inc.; Roche Molecular Biochemicals; and Promega Corp.

Liposomes may comprise cationic lipids alone, or in admixture with other lipids, particularly neutral lipids such as: cholesterol; 1,2-diacyl-sn-glycero-3-phosphoethanolamines, (including but not limited to dioleoyl (DOPE), 1,2-diacyl-sn-glycero-3-phosphocholines; natural egg yolk phosphatidyl choline (PC), and the like; synthetic mono- and diacyl phosphocholines (e.g., monoacyl phosphatidyl choline (MOPC)) and phosphoethanolamines. Asymmetric fatty acids, both synthetic and natural, and mixed formulations, for the above diacyl derivatives may also be included.

Other suitable liposome compositions include dimyristoylphosphatidylcholine (DMPC) and cholesterol. Such liposomes are described in, e.g., U.S. Pat. No. 5,916,588. Additional suitable liposomal compositions, and methods of preparing same, are known in the art, and are described in various publications, including, e.g., U.S. Pat. Nos. 4,241,046 and 6,355,267.

### Line Polynucleotides

The present invention provides a recombinant (e.g., synthetic) nucleic acid comprises a nucleotide sequence encoding a subject LINE polypeptide. A recombinant (e.g., synthetic) nucleic acid comprising a nucleotide sequence encoding a subject LINE polypeptide is referred to herein as a “subject LINE nucleic acid” or a “subject LINE polynucleotide.” The present invention further provides compositions, including pharmaceutical compositions and immunogenic compositions, comprising a subject LINE polynucleotide.

In certain embodiments, a subject LINE polynucleotide comprises a nucleotide sequence encoding subject LINE polypeptide, where the LINE polypeptide comprises an amino acid sequence having at least about 75%, at least about 80%, at least about 85%, at least about 90%, at least about 95%, at least about 98%, at least about 99%, or 100%, amino acid sequence identity to the amino acid sequence as set forth in any one of SEQ ID NOs:1-22.

In some embodiments, a subject LINE nucleic acid comprises a nucleotide sequence encoding a single type (or “species”) of LINE polypeptide, e.g., in some embodiments, the LINE nucleic acids all comprise nucleotide sequences substantially the same amino acid sequence. In other embodiments, a subject LINE nucleic acid composition comprises two or more different LINE nucleic acids, e.g., the composition comprises a population of LINE nucleic acids encoding a population of LINE polypeptides, the members of which population can differ in amino acid sequence. A population of encoded LINE polypeptides can comprise from two to about 20 different LINE polypeptides, e.g., a subject composition can comprise two, three, four, five, six, seven, eight, nine, ten, 11-15, or 15-20 different LINE polypeptides, each having an amino acid that differs from the amino acid sequences of the other LINE polypeptides. For example, in some embodiments, a population of encoded LINE polypeptides comprises a first LINE polypeptide having a first amino acid sequence; and at least a second LINE polypeptide having a second amino acid sequence, where the second amino acid sequence differs from the first amino acid sequence. As another example, in some embodiments, a population of encoded LINE polypeptides a first LINE polypeptide having a first amino acid sequence; second LINE polypeptide having a second amino acid sequence, where the second amino acid sequence differs from the first amino acid sequence; and at least a third LINE polypeptide having a third amino acid sequence, where the third amino acid sequence differs from both the first and the second amino acid sequences. In other embodiments, the encoded LINE polypeptide is a multimerized LINE polypeptide, as described above.

### Expression Vectors and Delivery Vehicles

In some embodiments, a subject LINE polynucleotide is an expression vector. The expression vector will provide a transcriptional and translational initiation region, which may be inducible or constitutive, where the coding region is operably linked under the transcriptional control of the transcriptional initiation region, and a transcriptional and translational termination region. Thus, e.g., a subject LINE polynucleotide can comprise a nucleotide sequence encoding a subject LINE polypeptide, where the LINE polypeptide-encoding nucleotide sequence is operably linked to a transcriptional control element (e.g., a promoter), where the transcriptional control element can be inducible or constitutive.

Expression vectors generally have convenient restriction sites located near the promoter sequence to provide for the insertion of nucleic acid sequences encoding heterologous proteins (e.g., to provide for insertion of a nucleotide sequence encoding a subject LINE polypeptide). A selectable marker operative in the expression host may be present. Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; L1 and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:8186, 1998, Flannery et al., PNAS 94:6916 6921, 1997; Bennett et al., Invest Opthalmol V is Sci 38:2857 2863, 1997; Jomary et al., Gene Ther 4:683 690, 1997, Rolling et al., Hum Gene Ther 10:641648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Numerous suitable expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

Non-limiting examples of suitable eukaryotic promoters (promoters functional in a eukaryotic cell) include CMV immediate early, HSV thymidine kinase, early and late SV40, LTRs from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

A subject recombinant vector will in some embodiments include one or more selectable markers. In addition, the expression vectors will in many embodiments contain one or more selectable marker genes to provide a phenotypic trait for selection of transformed host cells such as dihydrofolate reductase or neomycin resistance for eukaryotic cell culture.

Other gene delivery vehicles and methods may be employed, including polycationic condensed DNA linked or unlinked to killed adenovirus alone, for example Curiel (1992) Hum. Gene Ther. 3:147-154; ligand linked DNA, for example see Wu (1989) J. Biol. Chem. 264:16985-16987; eukaryotic cell delivery vehicles cells; deposition of photopolymerized hydrogel materials; hand-held gene transfer particle gun, as described in U.S. Pat. No. 5,149,655; ionizing radiation as described in U.S. Pat. No. 5,206,152 and in WO 92/11033; nucleic charge neutralization or fusion with cell membranes. Additional approaches are described in Philip (1994) Mol. Cell. Biol. 14:2411-2418, and in Woffendin (1994) Proc. Natl. Acad. Sci. 91:1581-1585.

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm. Liposomes that can act as gene delivery vehicles are described in U.S. Pat. No. 5,422,120, PCT Nos. WO 95/13796, WO 94/23697, and WO 91/14445, and EP No. 524 968.

Liposome or lipid nucleic acid delivery vehicles can also be used. Liposome complexes for gene delivery are described in, e.g., U.S. Pat. No. 7,001,614. For example, liposomes comprising DOTAP and at least one cholesterol and/or cholesterol-derivative, present in a molar ratio range of 2.0 mM 10 mM provide an effective delivery system, e.g., where the molar ratio of DOTAP to cholesterol is 1:1 3:1. The cationic lipid N-[(2,3-dioleoyloxy)propyl]-L-lysinamide (LADOP) can be used in a composition for delivering a LINE polynucleotide, where LADOP-containing liposomes are described in, e.g., U.S. Pat. No. 7,067,697. Liposome formulations comprising amphipathic lipids having a polar headgroup and aliphatic components capable of promoting transfection are suitable for use and are described in, e.g., U.S. Pat. No. 6,433,017.

Further non-viral delivery suitable for use includes mechanical delivery systems such as the approach described in Woffendin et al, (1994) Proc. Natl. Acad. Sci. USA 91:11581-11585. Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and PCT No. WO 92/11033.

### Compositions

The present invention provides compositions comprising a subject LINE nucleic acid. Compositions comprising a subject LINE nucleic acid can include one or more of: a salt, e.g., NaCl, MgCl, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a nuclease inhibitor; and the like. In some embodiments, as described in more detail below, a subject LINE nucleic acid composition is an immunogenic composition.

### Pharmaceutical Compositions

The present invention provides a pharmaceutical composition comprising a subject LINE nucleic acid and a pharmaceutically acceptable excipient. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents.

### Immunogenic Compositions

The present invention provides an immunogenic composition comprising a subject LINE polynucleotide. When administered to an individual in need thereof, a subject LINE polynucleotide is taken up by a cell, e.g., an antigen-presenting cell, the encoded LINE polypeptide is produced in the cell, and the LINE polypeptide is processed into polypeptide fragments (“epitope fragments”) that are then displayed on the surface of the cell in association with an MHC molecule. The encoded LINE polypeptide stimulates or enhances a T cell response to the epitope(s) displayed on the cell surface. Where the LINE epitopes are also present on a retrovirus-infected cell, a T cell response to the retrovirus-infected cell also occurs.

A subject immunogenic composition comprising a subject LINE nucleic acid includes, in addition to a subject LINE nucleic acid, one or more additional components, as described above for immunogenic compositions comprising a subject LINE polypeptide.

### Adjuvants

In some embodiments, a subject immunogenic composition comprises a subject LINE polynucleotide and an adjuvant. Suitable adjuvants include those suitable for use in humans. Examples of known suitable adjuvants that can be used in humans include, but are not necessarily limited to, alum, aluminum phosphate, aluminum hydroxide, MF59 (4.3% w/v squalene, 0.5% w/v polysorbate 80 (Tween 80), 0.5% w/v sorbitan trioleate (Span 85)), a CpG-containing nucleic acid (where the cytosine is unmethylated), QS21 (saponin adjuvant), MPL (Monophosphoryl Lipid A), 3DMPL (3-O-deacylated MPL), extracts from Aquilla, ISCOMS (see, e.g., Sjölander et al. (1998) J. Leukocyte Biol. 64:713), LT/CT mutants, poly(D,L-lactide-co-glycolide) (PLG) microparticles, Quil A, interleukins, and the like. For veterinary applications including but not limited to animal experimentation, one can use Freund's, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIM, which contains three components extracted from bacteria, monophosphoryl lipid A, trehalose dimycolate and cell wall skeleton (MPL+TDM+CWS) in a 2% squalene/Tween 80 emulsion.

Further exemplary adjuvants to enhance effectiveness of the composition include, but are not limited to: (1) oil-in-water emulsion formulations (with or without other specific immunostimulating agents such as muramyl peptides (see below) or bacterial cell wall components), such as for example (a) MF59™ (WO90/14837; Chapter 10 in Vaccine design: the subunit and adjuvant approach, eds. Powell & Newman, Plenum Press 1995), containing 5% Squalene, 0.5% Tween 80 (polyoxyethylene sorbitan mono-oleate), and 0.5% Span 85 (sorbitan trioleate) (optionally containing muramyl tri-peptide covalently linked to dipalmitoyl phosphatidylethanolamine (MTP-PE)) formulated into submicron particles using a microfluidizer, (b) SAF, containing 10% Squalane, 0.4% Tween 80, 5% pluronic-blocked polymer L121, and thr-MDP either microfluidized into a submicron emulsion or vortexed to generate a larger particle size emulsion, and (c) RIBI™ adjuvant system (RAS), (Ribi Immunochem, Hamilton, Mont.) containing 2% Squalene, 0.2% Tween 80, and one or more bacterial cell wall components such as monophosphorylipid A (MPL), trehalose dimycolate (TDM), and cell wall skeleton (CWS), e.g., MPL+CWS (DETOX™); (2) saponin adjuvants, such as QS21 or STIMULON™ (Cambridge Bioscience, Worcester, Mass.) may be used or particles generated therefrom such as ISCOMs (immunostimulating complexes), which ISCOMS may be devoid of additional detergent e.g. WO00/07621; (3) Complete Freund's Adjuvant (CFA) and Incomplete Freund's Adjuvant (IFA); (4) cytokines, such as interleukins (e.g. IL-1, IL-2, IL-4, IL-5, IL-6, IL-7, IL-12 (WO99/44636), etc.), interferons (e.g. gamma interferon), macrophage colony stimulating factor (M-CSF), tumor necrosis factor (TNF), other TNF superfamily molecules (e.g., CH40L, OX40L, and the like), etc.; (5) monophosphoryl lipid A (MPL) or 3-O-deacylated MPL (3dMPL) e.g. GB-2220221, EP-A-0689454, optionally in the substantial absence of alum when used with pneumococcal saccharides e.g. WO00/56358; (6) combinations of 3dMPL with, for example, QS21 and/or oil-in-water emulsions e.g. EP-A-0835318, EP-A-0735898, EP-A-0761231; (7) oligonucleotides comprising CpG motifs [Krieg Vaccine 2000, 19, 618-622; Krieg Curr Opin Mol Ther2001 3:15-24; Roman et al., Nat. Med., 1997, 3, 849-854; Weiner et al., PNAS USA, 1997, 94, 10833-10837; Davis et al, J. Immunol, 1998, 160, 870-876; Chu et al., J. Exp. Med, 1997, 186, 1623-1631; Lipford et al, Eur. J. Immunol., 1997, 27, 2340-2344; Moldoveanu et al., Vaccine, 1988, 16, 1216-1224, Krieg et al., Nature, 1995, 374, 546-549; Klinman et al., PNAS USA, 1996, 93, 2879-2883; Ballas et al, J. Immunol, 1996, 157, 1840-1845; Cowdery et al, J. Immunol, 1996, 156, 4570-4575; Halpern et al, Cell Immunol, 1996, 167, 72-78; Yamamoto et al, Jpn. J. Cancer Res., 1988, 79, 866-873; Stacey et al, J. Immunol., 1996, 157, 2116-2122; Messina et al, J. Immunol, 1991, 147, 1759-1764; Yi et al, J. Immunol, 1996, 157, 4918-4925; Yi et al, J. Immunol, 1996, 157, 5394-5402; Yi et al, J. Immunol, 1998, 160, 4755-4761; and Yi et al, J. Immunol, 1998, 160, 5898-5906; International patent applications WO96/02555, WO98/16247, WO98/18810, WO98/40100, WO98/55495, WO98/37919 and WO98/52581]i.e. containing at least one CG dinucleotide, where the cytosine is unmethylated; (8) a polyoxyethylene ether or a polyoxyethylene ester e.g. WO99/52549; (9) a polyoxyethylene sorbitan ester surfactant in combination with an octoxynol (WO01/21207) or a polyoxyethylene alkyl ether or ester surfactant in combination with at least one additional non-ionic surfactant such as an octoxynol (WO01/21152); (10) a saponin and an immunostimulatory oligonucleotide (e.g. a CpG oligonucleotide) (WO00/62800); (11) an immunostimulant and a particle of metal salt e.g. WO00/23105; (12) a saponin and an oil-in-water emulsion e.g. WO99/11241; (13) a saponin (e.g. QS21)+3dMPL+IM2 (optionally+a sterol) e.g. WO98/57659; (14) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutaminyl-L-alanine-2-(1′-2′-dipalmitoyl-sn-glycero-3-hydroxyphosphoryloxy)-ethylamine MTP-PE), etc.

A subject immunogenic composition can include a conventional pharmaceutically acceptable excipient, such as pharmaceutical grades of mannitol, lactose, starch, magnesium stearate, sodium saccharin, talcum, cellulose, glucose, sucrose, magnesium, carbonate, and the like. A subject immunogenic composition can include one or more pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of a subject LINE nucleic acid in these formulations can vary widely, and can be selected based on various factors such as fluid volumes, viscosities, body weight and the like in accordance with the particular mode of administration selected and the patient's needs. The resulting compositions may be in the form of a solution, suspension, tablet, pill, capsule, powder, gel, cream, lotion, ointment, aerosol or the like.

The concentration of a subject LINE polynucleotide in the pharmaceutical formulations can vary widely, e.g., less than about 0.1%, from about 0.1% to about 2%, from about 2% to 20%, or from about 20% to about 50%, or more, by weight, and will be selected on the basis of various factors such as fluid volumes, viscosities, etc., in accordance with the particular mode of administration selected.

In some embodiments, a subject LINE polynucleotide is formulated with one or more lipids. For example, liposomes of various sizes can be made. Small liposomes or vesicles formed are unilamellar and have a size in the range of about 20 to 400 nanometers and can be produced by subjecting multi-lamellar vesicles to ultrasound, by extrusion under pressure through membranes having pores of defined size, or by high pressure homogenization. Larger unilamellar liposomes having a size in the range of about 0.1 to 1 μm in diameter can be obtained when the lipid is solubilized in an organic solvent or a detergent and the solubilized agent is removed by evaporation or dialysis, respectively. The fusion of smaller unilamellar liposomes by methods requiring particular lipids or stringent dehydration-hydration conditions can yield unilamellar vessels as large as or larger than cells.

Liposomes can comprise one or more cationic lipids, e.g., DDAB, dimethyldioctadecyl ammonium bromide; N-[1-(2,3-Dioloyloxy)propyl]-N,N,N-trimethylammonium methylsulfate; 1,2-diacyl-3-trimethylammonium-propanes, (including but not limited to, dioleoyl (DOTAP), dimyristoyl, dipalmitoyl, disearoyl); 1,2-diacyl-3-dimethylammonium-propanes, (including but not limited to, dioleoyl, dimyristoyl, dipalmitoyl, disearoyl) DOTMA, N-[1-[2,3-bis(oleoyloxy)]propyl]-N,N,N-trimethylammonium chloride; DOGS, dioctadecylamidoglycylspermine; DC-cholesterol, 3β[N-(N′,N′-dimethylaminoethane)carbamoyl]cholesterol; DOSPA, 2,3-dioleoyloxy-N-(2(sperminecarboxamido)-ethyl)-N,N-dimethyl-1-propanaminium trifluoroacetate; 1,2-diacyl-sn-glycero-3-ethylphosphocholines (including but not limited to dioleoyl (DOEPC), dilauroyl, dimyristoyl, dipalmitoyl, distearoyl, palmitoyl-oleoyl); β-alanyl cholesterol; CTAB, cetyl trimethyl ammonium bromide; diC14-amidine, N-t-butyl-N′-tetradecyl-3-tetradecylaminopropionamidine; 14Dea2, O,O′-ditetradecanolyl-N-(trimethylammonioacetyl)diethanolamine chloride; DOSPER, 1,3-dioleoyloxy-2-(6-carboxy-spermyl)-propylamide; N,N,N′,N′-tetramethyl-N,N′-bis(2-hydroxylethyl)-2,3-dioleoyloxy-1,4-butanediammonium iodide; 1-[2-acyloxy)ethyl]2-alkyl(alkenyl)-3-(2-hydroxyethyl)imidazolinium chloride derivatives such as 1-[2-(9(Z)-octadecenoyloxy)ethyl]-2-(8(Z)-heptadecenyl-3-(2-hydroxyethyl)imidazolinium chloride (DOTIM), 1-[2-(hexadecanoyloxy)ethyl]-2-pentadecyl-3-(2-hydroxyethyl)imidazolinium chloride (DPTIM); 1-[2-tetradecanoyloxy)ethyl]-2-tridecyl-3-(2-hydroxyethyl)imidazolium chloride (DMTIM)—as described in Solodin et al. (1995) Biochem. 43:13537-13544; 2,3-dialkyloxypropyl quaternary ammonium compound derivates, containing a hydroxyalkyl moiety on the quaternary amine, such as 1,2-dioleoyl-3-dimethyl-hydroxyethyl ammonium bromide (DORI); 1,2-dioleyloxypropyl-3-dimethyl-hydroxyethyl ammonium bromide (DOME); 1,2-dioleyloxypropyl-3-dimethyl-hydroxypropyl ammonium bromide (DORIE-HP); 1,2-dioleyloxypropyl-3-dimethyl-hydroxybutyl ammonium bromide (DORIE-HB); 1,2-dioleyloxypropyl-3-dimethyl-hydroxypentyl ammonium bromide (DORIE-HPe); 1,2-dimyristyloxypropyl-3-dimethyl-hydroxylethyl ammonium bromide (DMRIE); 1,2-dipalmityloxypropyl-3-dimethyl-hydroxyethyl ammonium bromide (DPRIE); 1,2-disteryloxypropyl-3-dimethyl-hydroxyethyl ammonium bromide (DSRIE)—as described, e.g., in Felgner et al. (1994)J. Biol. Chem. 269:2550-2561. Many of the above-mentioned lipids are available commercially from, e.g., Avanti Polar Lipids, Inc.; Sigma Chemical Co.; Molecular Probes, Inc.; Northerm Lipids, Inc.; Roche Molecular Biochemicals; and Promega Corp.

Liposomes may comprise cationic lipids alone, or in admixture with other lipids, particularly neutral lipids such as: cholesterol; 1,2-diacyl-sn-glycero-3-phosphoethanolamines, (including but not limited to dioleoyl (DOPE), 1,2-diacyl-sn-glycero-3-phosphocholines; natural egg yolk phosphatidyl choline (PC), and the like; synthetic mono- and diacyl phosphocholines (e.g., monoacyl phosphatidyl choline (MOPC)) and phosphoethanolamines. Asymmetric fatty acids, both synthetic and natural, and mixed formulations, for the above diacyl derivatives may also be included.

Other suitable liposome compositions include dimyristoylphosphatidylcholine (DMPC) and cholesterol. Such liposomes are described in, e.g., U.S. Pat. No. 5,916,588. Additional suitable liposomal compositions, and methods of preparing same, are known in the art, and are described in various publications, including, e.g., U.S. Pat. Nos. 4,241,046 and 6,355,267.

### Treatment Methods

A variety of treatment methods are contemplated by the present disclosure, which methods utilize a subject LINE polypeptide, a subject LINE polynucleotide, or a subject LINE composition. Subject treatment methods include methods of inducing an immune response in an individual to a LINE polypeptide, and methods of enhancing a subject's immune response to a LINE polypeptide, e.g., for the treatment of a retrovirus infection (e.g., a lentivirus infection), for the treatment of cancer, etc; and methods for reducing subject's immune response to a LINE polypeptide, e.g., for the treatment of an autoimmune disorder, for the treatment of schizophrenia, etc.

### Methods of Inducing or Enhancing an Immune Response to a Retrovirus-Infected Cell

The present disclosure provides methods for inducing, eliciting, or enhancing a T cell immune response to a retrovirus-infected cell, e.g., an HTLV-infected cell, in an individual in need thereof. The methods generally involve administering an effective amount of a subject LINE polypeptide, a subject LINE polynucleotide, or a subject LINE composition (e.g., a subject LINE immunogenic composition) to the individual.

Thus, e.g., the present invention provides methods for treating a retrovirus infection in an individual, the methods generally involving administering to an individual in need thereof an effective amount of a subject LINE polypeptide (e.g. a subject isolated LINE polypeptide, a subject synthetic LINE polypeptide), a subject LINE polynucleotide, or a subject LINE composition (e.g., a subject LINE pharmaceutical composition, a subject LINE immunogenic composition). In some embodiments, the present invention provides methods for treating a retrovirus infection in an individual, the methods generally involving administering to an individual in need thereof an effective amount of a subject LINE immunogenic composition, e.g., a subject immunogenic composition comprising a subject LINE polypeptide or a subject LINE polynucleotide. The present invention provides use of a subject LINE polypeptide, a subject LINE polynucleotide, or a subject LINE composition in the preparation of a medicament for the treatment of a retrovirus infection in an individual. The present invention provides use of a subject LINE immunogenic composition (e.g., a subject immunogenic composition comprising a subject LINE polypeptide or a subject LINE polynucleotide) in the preparation of a medicament for the treatment of a retrovirus infection in an individual. The present invention provides a subject LINE polypeptide, a subject LINE polynucleotide, or a subject LINE composition for treating a retrovirus infection in an individual. The present invention provides a subject LINE immunogenic composition (e.g., a subject immunogenic composition comprising a subject LINE polypeptide or a subject LINE polynucleotide) for treating a retrovirus infection in an individual.

Thus, e.g., the present invention provides methods for treating an HTLV infection in an individual, the methods generally involving administering to an individual in need thereof an effective amount of a subject LINE polypeptide (e.g. a subject isolated LINE polypeptide, a subject synthetic LINE polypeptide), a subject LINE polynucleotide, or a subject LINE composition (e.g., a subject LINE pharmaceutical composition, a subject LINE immunogenic composition). In some embodiments, the present invention provides methods for treating an HTLV infection in an individual, the methods generally involving administering to an individual in need thereof an effective amount of a subject LINE immunogenic composition, e.g., a subject immunogenic composition comprising a subject LINE polypeptide or a subject LINE polynucleotide. The present invention provides use of a subject LINE polypeptide, a subject LINE polynucleotide, or a subject LINE composition in the preparation of a medicament for the treatment of an HTLV infection in an individual. The present invention provides use of a subject LINE immunogenic composition (e.g., a subject immunogenic composition comprising a subject LINE polypeptide or a subject LINE polynucleotide) in the preparation of a medicament for the treatment of an HTLV infection in an individual. The present invention provides a subject LINE polypeptide, a subject LINE polynucleotide, or a subject LINE composition for treating an HTLV infection in an individual. The present invention provides a subject LINE immunogenic composition (e.g., a subject immunogenic composition comprising a subject LINE polypeptide or a subject LINE polynucleotide) for treating an HTLV infection in an individual.

In some embodiments, an “effective amount” of a subject LINE polypeptide, a subject LINE polynucleotide, or a subject LINE composition is an amount that, when administered to an individual in one or more doses, reduces retroviral load (e.g., HTLV load) in the individual by at least about 5%, at least about 10%, at least about 20%, at least about 25%, at least about 50%, at least about 75%, at least about 85%, or at least about 90%, compared to the viral load in the individual before treatment with the subject LINE polypeptide, the subject LINE polynucleotide, or the subject LINE composition.

In some embodiments, a subject method of inducing, eliciting, or enhancing a T cell immune response to a retrovirus-infected cell involves administering to an individual in need thereof an effective amount of a subject immunogenic composition. In some embodiments, an “effective amount” of a subject immunogenic composition is an amount that, when administered to an individual in one or more doses, reduces retroviral load in the individual by at least about 5%, at least about 10%, at least about 20%, at least about 25%, at least about 50%, at least about 75%, at least about 85%, or at least about 90%, compared to the viral load in the individual before treatment with the immunogenic composition. In some embodiments, the immunogenic composition comprises a subject LINE polypeptide. In other embodiments, the immunogenic composition comprises a subject LINE polynucleotide.

In some embodiments, an “effective amount” of a subject LINE polypeptide, a subject LINE polynucleotide, or a subject LINE composition is an amount that, when administered to an individual in one or more doses, results in an increase in the number of T cells specific for a retrovirus epitope present on a retrovirus-infected cell. In some embodiments, an “effective amount” of a subject LINE polypeptide, a subject LINE polynucleotide, or a subject LINE composition is an amount that, when administered to an individual in one or more doses, results in an increase of at least about 25%, at least about 50%, at least about 100% or 2-fold, at least about 5-fold, at least about 10-fold, or at least about 100-fold, or more, in the number of T cells specific for a retrovirus epitope present on a retrovirus-infected cell, compared with the number of T cells specific for a retrovirus epitope in the individual before treatment with the subject LINE polypeptide, the subject LINE polynucleotide, or the subject LINE composition.

In some embodiments, an “effective amount” of a subject immunogenic composition is an amount that, when administered to an individual in one or more doses, results in an increase in the number of T cells specific for a retrovirus epitope present on a retrovirus-infected cell. In some embodiments, an “effective amount” of a subject immunogenic composition is an amount that, when administered to an individual in one or more doses, results in an increase of at least about 25%, at least about 50%, at least about 100% or 2-fold, at least about 5-fold, at least about 10-fold, or at least about 100-fold, or more, in the number of T cells specific for a retrovirus epitope present on a retrovirus-infected cell, compared with the number of T cells specific for a retrovirus epitope in the individual before treatment with the immunogenic composition. In some embodiments, the immunogenic composition comprises a subject LINE polypeptide. In other embodiments, the immunogenic composition comprises a subject LINE polynucleotide.

In some embodiments, an “effective amount” of a subject LINE polypeptide, a subject LINE polynucleotide, or a subject LINE composition is an amount that, when administered to an individual in one or more doses, results in an increase in the number of CD8⁺ T cells specific for a retrovirus epitope present on a retrovirus-infected cell. In some embodiments, an “effective amount” of a subject LINE polypeptide, a subject LINE polynucleotide, or a subject LINE composition is an amount that, when administered to an individual in one or more doses, results in an increase of at least about 25%, at least about 50%, at least about 100% or 2-fold, at least about 5-fold, at least about 10-fold, or at least about 100-fold, or more, in the number of CD8⁺ T cells specific for a retrovirus epitope present on a retrovirus-infected cell, compared with the number of CD8⁺ T cells specific for a retrovirus epitope in the individual before treatment with the subject LINE polypeptide, the subject LINE polynucleotide, or the subject LINE composition.

In some embodiments, an “effective amount” of a subject immunogenic composition is an amount that, when administered to an individual in one or more doses, results in an increase in the number of CD8⁺ T cells specific for a retrovirus epitope present on a retrovirus-infected cell. In some embodiments, an “effective amount” of a subject immunogenic composition is an amount that, when administered to an individual in one or more doses, results in an increase of at least about 25%, at least about 50%, at least about 100% or 2-fold, at least about 5-fold, at least about 10-fold, or at least about 100-fold, or more, in the number of CD8⁺ T cells specific for a retrovirus epitope present on a retrovirus-infected cell, compared with the number of CD8⁺ T cells specific for a retrovirus epitope in the individual before treatment with the immunogenic composition. In some embodiments, the immunogenic composition comprises a subject LINE polypeptide. In other embodiments, the immunogenic composition comprises a subject LINE polynucleotide.

In some embodiments, e.g., where a subject LINE polypeptide, a subject LINE polynucleotide, or a subject LINE composition is administered to a naïve individual (i.e., an individual not infected with a retrovirus such as HTLV), an “effective amount” of a subject LINE polypeptide, a subject LINE polynucleotide, or a subject LINE composition is an amount that, when administered to an individual in one or more doses, reduces the likelihood that the individual, if later infected with a retrovirus such as HTLV, would develop disease symptoms from the retrovirus infection. In some embodiments, e.g., where a subject LINE polypeptide, a subject LINE polynucleotide, or a subject LINE composition is administered to a naïve individual (i.e., an individual not infected with a retrovirus), an “effective amount” of a subject LINE polypeptide, a subject LINE polynucleotide, or a subject LINE composition is an amount that, when administered to an individual in one or more doses, increases the likelihood that the individual, if later infected with a retrovirus such as HIV, would limit and/or clear the retrovirus infection.

In some embodiments, e.g., where a subject immunogenic composition is administered to a naïve individual (i.e., an individual not infected with a retrovirus such as HTLV), an “effective amount” of a subject immunogenic composition is an amount that, when administered to an individual in one or more doses, reduces the likelihood that the individual, if later infected with a retrovirus such as HTLV, would develop disease symptoms from the retrovirus infection. In some embodiments, e.g., where the immunogenic composition is administered to a naïve individual (i.e., an individual not infected with a retrovirus), an “effective amount” of a subject immunogenic composition is an amount that, when administered to an individual in one or more doses, increases the likelihood that the individual, if later infected with a retrovirus such as HIV, would limit and/or clear the retrovirus infection.

### Methods of Inducing or Enhancing an Immune Response to a Lentivirus-Infected Cell

The present invention provides methods for inducing, eliciting, or enhancing a T cell immune response to a lentivirus-infected cell, e.g., an HIV-infected cell, in an individual in need thereof. The methods generally involve administering an effective amount of a subject LINE polypeptide, a subject LINE polynucleotide, or a subject LINE composition to the individual.

The present invention provides methods for treating a lentivirus infection (e.g., an HIV infection) in an individual, the methods generally involving administering to an individual in need thereof an effective amount of a subject LINE polypeptide (e.g. a subject isolated LINE polypeptide, a subject synthetic LINE polypeptide), a subject LINE polynucleotide, or a subject LINE composition (e.g., a subject LINE pharmaceutical composition, a subject LINE immunogenic composition). In some embodiments, the present invention provides methods for treating a lentivirus infection in an individual, the methods generally involving administering to an individual in need thereof an effective amount of a subject LINE immunogenic composition, e.g., a subject immunogenic composition comprising a subject LINE polypeptide or a subject LINE polynucleotide. The present invention provides use of a subject LINE polypeptide, a subject LINE polynucleotide, or a subject LINE composition in the preparation of a medicament for the treatment of a lentivirus infection in an individual. The present invention provides use of a subject LINE immunogenic composition (e.g., a subject immunogenic composition comprising a subject LINE polypeptide or a subject LINE polynucleotide) in the preparation of a medicament for the treatment of a lentivirus infection in an individual. The present invention provides a subject LINE polypeptide, a subject LINE polynucleotide, or a subject LINE composition for treating a lentivirus infection in an individual. The present invention provides a subject LINE immunogenic composition (e.g., a subject immunogenic composition comprising a subject LINE polypeptide or a subject LINE polynucleotide) for treating a lentivirus infection in an individual.

The present invention provides methods for treating an HIV infection in an individual, the methods generally involving administering to an individual in need thereof an effective amount of a subject LINE polypeptide (e.g. a subject isolated LINE polypeptide, a subject synthetic LINE polypeptide), a subject LINE polynucleotide, or a subject LINE composition (e.g., a subject LINE pharmaceutical composition, a subject LINE immunogenic composition). In some embodiments, the present invention provides methods for treating an HIV infection in an individual, the methods generally involving administering to an individual in need thereof an effective amount of a subject LINE immunogenic composition, e.g., a subject immunogenic composition comprising a subject LINE polypeptide or a subject LINE polynucleotide. The present invention provides use of a subject LINE polypeptide, a subject LINE polynucleotide, or a subject LINE composition in the preparation of a medicament for the treatment of an HIV infection in an individual. The present invention provides use of a subject LINE immunogenic composition (e.g., a subject immunogenic composition comprising a subject LINE polypeptide or a subject LINE polynucleotide) in the preparation of a medicament for the treatment of an HIV infection in an individual. The present invention provides a subject LINE polypeptide, a subject LINE polynucleotide, or a subject LINE composition for treating an HIV infection in an individual. The present invention provides a subject LINE immunogenic composition (e.g., a subject immunogenic composition comprising a subject LINE polypeptide or a subject LINE polynucleotide) for treating an HIV infection in an individual.

In some embodiments, an “effective amount” of a subject LINE polypeptide, a subject LINE polynucleotide, or a subject LINE composition is an amount that, when administered to an individual in one or more doses, reduces viral load (e.g., HIV viral load) in the individual by at least about 5%, at least about 10%, at least about 20%, at least about 25%, at least about 50%, at least about 75%, at least about 85%, or at least about 90%, compared to the viral load in the individual before treatment with the subject LINE polypeptide, the subject LINE polynucleotide, or the subject LINE composition.

In some embodiments, a subject method for inducing, eliciting, or enhancing a T cell immune response to a lentivirus-infected cell in an individual involves administering to the individual an effective amount of a subject immunogenic composition. In some embodiments, an “effective amount” of a subject immunogenic composition is an amount that, when administered to an individual in one or more doses, reduces viral load in the individual by at least about 5%, at least about 10%, at least about 20%, at least about 25%, at least about 50%, at least about 75%, at least about 85%, or at least about 90%, compared to the viral load in the individual before treatment with the immunogenic composition. In some embodiments, the immunogenic composition comprises a subject LINE polypeptide. In other embodiments, the immunogenic composition comprises a subject LINE polynucleotide.

In some embodiments, an “effective amount” of a subject LINE polypeptide, a subject LINE polynucleotide, or a subject LINE composition is an amount that, when administered to an individual in one or more doses, results in an increase in CD4⁺ T lymphocyte levels and function(s) in the individual. In some embodiments, an “effective amount” of a subject LINE polypeptide, a subject LINE polynucleotide, or a subject LINE composition is an amount that, when administered to an individual in one or more doses, results in an increase of at least about 25%, at least about 50%, at least about 100% or 2-fold, at least about 5-fold, at least about 10-fold, or at least about 100-fold, or more, compared to the level of CD4⁺ T lymphocytes in the individual before treatment with the subject LINE polypeptide, the subject LINE polynucleotide, or the subject LINE composition. In some embodiments, an “effective amount” of a subject LINE polypeptide, a subject LINE polynucleotide, or a subject LINE composition is an amount that, when administered to an individual in one or more doses, results in a number of CD4⁺ T lymphocytes that is within the normal range, where the normal range for humans is from about 600 to about 1500 CD4⁺ T lymphocytes per mm³ blood.

In some embodiments, an “effective amount” of a subject immunogenic composition is an amount that, when administered to an individual in one or more doses, results in an increase in CD4⁺ T lymphocyte levels and function(s) in the individual. In some embodiments, an “effective amount” of a subject immunogenic composition is an amount that, when administered to an individual in one or more doses, results in an increase of at least about 25%, at least about 50%, at least about 100% or 2-fold, at least about 5-fold, at least about 10-fold, or at least about 100-fold, or more, compared to the level of CD4⁺ T lymphocytes in the individual before treatment with the immunogenic composition. In some embodiments, an “effective amount” of a subject immunogenic composition is an amount that, when administered to an individual in one or more doses, results in a number of CD4⁺ T lymphocytes that is within the normal range, where the normal range for humans is from about 600 to about 1500 CD4⁺ T lymphocytes per mm³ blood. In some embodiments, the immunogenic composition comprises a subject LINE polypeptide. In other embodiments, the immunogenic composition comprises a subject LINE polynucleotide.

In some embodiments, an “effective amount” of a subject LINE polypeptide, a subject LINE polynucleotide, or a subject LINE composition is an amount that, when administered to an individual in one or more doses, results in an increase in the number of T cells specific for a lentivirus epitope (e.g., an HIV epitope) present on a lentivirus-infected cell (e.g., an HIV-infected cell). In some embodiments, an “effective amount” of a subject LINE polypeptide, a subject LINE polynucleotide, or a subject LINE composition is an amount that, when administered to an individual in one or more doses, results in an increase of at least about 25%, at least about 50%, at least about 100% or 2-fold, at least about 5-fold, at least about 10-fold, or at least about 100-fold, or more, in the number of T cells specific for a lentivirus epitope (e.g., an HIV epitope) present on a lentivirus-infected cell (e.g., an HIV-infected cell), compared with the number of T cells specific for a lentivirus epitope in the individual before treatment with the subject LINE polypeptide, the subject LINE polynucleotide, or the subject LINE composition.

In some embodiments, an “effective amount” of a subject immunogenic composition is an amount that, when administered to an individual in one or more doses, results in an increase in the number of T cells specific for a lentivirus epitope present on a lentivirus-infected cell. In some embodiments, an “effective amount” of a subject immunogenic composition is an amount that, when administered to an individual in one or more doses, results in an increase of at least about 25%, at least about 50%, at least about 100% or 2-fold, at least about 5-fold, at least about 10-fold, or at least about 100-fold, or more, in the number of T cells specific for a lentivirus epitope present on a lentivirus-infected cell, compared with the number of T cells specific for a lentivirus epitope in the individual before treatment with the immunogenic composition. In some embodiments, the immunogenic composition comprises a subject LINE polypeptide. In other embodiments, the immunogenic composition comprises a subject LINE polynucleotide.

In some embodiments, an “effective amount” of a subject LINE polypeptide, a subject LINE polynucleotide, or a subject LINE composition is an amount that, when administered to an individual in one or more doses, results in an increase in the number of CD8⁺ T cells specific for a lentivirus epitope present on a lentivirus-infected cell. In some embodiments, an “effective amount” of a subject LINE polypeptide, a subject LINE polynucleotide, or a subject LINE composition is an amount that, when administered to an individual in one or more doses, results in an increase of at least about 25%, at least about 50%, at least about 100% or 2-fold, at least about 5-fold, at least about 10-fold, or at least about 100-fold, or more, in the number of CD8⁺ T cells specific for a lentivirus epitope present on a lentivirus-infected cell, compared with the number of CD8⁺ T cells specific for a lentivirus epitope in the individual before treatment with the subject LINE polypeptide, the subject LINE polynucleotide, or the subject LINE composition.

In some embodiments, an “effective amount” of a subject immunogenic composition is an amount that, when administered to an individual in one or more doses, results in an increase in the number of CD8⁺ T cells specific for a lentivirus epitope present on a lentivirus-infected cell. In some embodiments, an “effective amount” of a subject immunogenic composition is an amount that, when administered to an individual in one or more doses, results in an increase of at least about 25%, at least about 50%, at least about 100% or 2-fold, at least about 5-fold, at least about 10-fold, or at least about 100-fold, or more, in the number of CD8⁺ T cells specific for a lentivirus epitope present on a lentivirus-infected cell, compared with the number of CD8⁺ T cells specific for a lentivirus epitope in the individual before treatment with the immunogenic composition.

In some embodiments, e.g., where a subject LINE polypeptide, a subject LINE polynucleotide, or a subject LINE composition is administered to a naïve individual (i.e., an individual not infected with a lentivirus such as HIV), an “effective amount” of a subject LINE polypeptide, a subject LINE polynucleotide, or a subject LINE composition is an amount that, when administered to an individual in one or more doses, reduces the likelihood that the individual, if later exposed to or infected with a lentivirus such as HIV, would develop disease symptoms from the lentivirus infection. In some embodiments, e.g., where a subject LINE polypeptide, a subject LINE polynucleotide, or a subject LINE composition is administered to a naïve individual (i.e., an individual not infected with a lentivirus such as HIV), an “effective amount” of a subject LINE polypeptide, a subject LINE polynucleotide, or a subject LINE composition is an amount that, when administered to an individual in one or more doses, increases the likelihood that the individual, if later infected with a lentivirus such as HIV, would limit and/or clear the lentivirus infection.

In some embodiments, e.g., where the immunogenic composition is administered to a naïve individual (i.e., an individual not infected with a lentivirus such as HIV), an “effective amount” of a subject immunogenic composition is an amount that, when administered to an individual in one or more doses, reduces the likelihood that the individual, if later exposed to or infected with a lentivirus such as HIV, would develop disease symptoms from the lentivirus infection. In some embodiments, e.g., where the immunogenic composition is administered to a naïve individual (i.e., an individual not infected with a lentivirus such as HIV), an “effective amount” of a subject immunogenic composition is an amount that, when administered to an individual in one or more doses, increases the likelihood that the individual, if later infected with a lentivirus such as HIV, would limit and/or clear the lentivirus infection.

### Combination Therapies

A subject LINE polypeptide, a subject LINE polynucleotide, or a subject LINE composition can be administered in conjunction with one or more therapeutic agents for the treatment of a lentiviral infection, or for the treatment of a disorder that may accompany a lentiviral infection (e.g., a bacterial infection, a fungal infection, and the like). Therapeutic agents beta-lactam antibiotics, tetracyclines, chloramphenicol, neomycin, gramicidin, bacitracin, sulfonamides, nitrofurazone, nalidixic acid, cortisone, hydrocortisone, betamethasone, dexamethasone, fluocortolone, prednisolone, triamcinolone, indomethacin, sulindac, acyclovir, amantadine, rimantadine, recombinant soluble CD4 (rsCD4), anti-receptor antibodies (e.g., for rhinoviruses), nevirapine, cidofovir (Vistide™), trisodium phosphonoformate (Foscamet™), famcyclovir, pencyclovir, valacyclovir, nucleic acid/replication inhibitors, interferon, zidovudine (AZT, Retrovir™), didanosine (dideoxyinosine, ddI, Videx™), stavudine (d4T, Zerit™), zalcitabine (dideoxycytosine, ddC, Hivid™), nevirapine (Viramune™), lamivudine (Epivir™, 3TC), protease inhibitors, saquinavir (Invirase™, Fortovase™), ritonavir (Norvir™), nelfinavir (Viracept™), efavirenz (Sustiva™), abacavir (Ziagen™), amprenavir (Agenerase™) indinavir (Crixivan™), ganciclovir, AzDU, delavirdine (Rescriptor™), kaletra, trizivir, rifampin, clathiromycin, erythropoietin, colony stimulating factors (G-CSF and GM-CSF), non-nucleoside reverse transcriptase inhibitors, nucleoside reverse transcriptase inhibitors, adriamycin, fluorouracil, methotrexate, asparaginase and combinations thereof.

Agents for treating an HIV infection that can be administered in combination with a subject LINE polypeptide, a subject LINE polynucleotide, or a subject LINE composition include, e.g., non-nucleoside reverse transcriptase inhibitors (e.g., Efavirenz, Nevirapine, Delavirdine, Etravirine), nucleoside analog reverse transcriptase inhibitors (e.g., Zidovudine, Didanosine, Zalcitabine, Stavudine, Lamivudine, Abacavir, Emtricitabine), nucleotide analog reverse transcriptase inhibitors (Tenofovir, Adefovir), inhibitors of an HIV protease (Saquinavir, Ritonavir, Indinavir, Nelfinavir, Amprenavir, Lopinavir, Fosamprenavir, Tipranavir, Darunavir), inhibitors of an HIV integrase (e.g., raltegravir, elvitegravir), inhibitors of HIV entry or fusion (e.g., Maraviroc, enfuvirtide), and maturation inhibitors (e.g., bevirimat, vivecon).

### Methods of Treating Cancer

The present invention further provides methods of treating cancer in an individual, where the cancerous state is associated with aberrant expression of a LINE polypeptide or increased expression of a LINE polypeptide, e.g., where the cancer comprises a cancer cell or a pre-cancerous cell that exhibits aberrant expression of a LINE polypeptide (e.g., expresses a LINE polypeptide at a level that is at least about 15%, at least about 25%, at least about 30%, at least about 40%, at least about 50%, at least about 75%, at least about 2-fold, at least about 5-fold, or at least about 10-fold, or more than 10-fold, higher than the level of LINE polypeptide expressed by a non-cancerous (normal) cell of the same cell type). Such cancers include, but are not limited to, melanoma, ovarian cancer, breast cancer, and testicular cancer (including teratoma, seminoma, and embryonal carcinoma or mixed tumors composed of one or more of these types). The methods generally involve administering to an individual in need thereof an effective amount of a subject LINE polypeptide (e.g., a subject isolated LINE polypeptide, a subject synthetic LINE polypeptide), a subject LINE polynucleotide, or a subject LINE composition (e.g., a subject LINE pharmaceutical composition, a subject LINE immunogenic composition). In some embodiments, the generally involve administering to an individual in need thereof an effective amount of a subject LINE immunogenic composition (e.g., a subject LINE immunogenic composition comprising one or more subject LINE polypeptides or one or more subject LINE polynucleotides).

The present invention provides methods for treating a cancer (e.g., melanoma, ovarian cancer, breast cancer, and testicular cancer (including teratoma, seminoma, and embryonal carcinoma or mixed tumors composed of one or more of these types) in an individual, the methods generally involving administering to an individual in need thereof an effective amount of a subject LINE polypeptide (e.g. a subject isolated LINE polypeptide, a subject synthetic LINE polypeptide), a subject LINE polynucleotide, or a subject LINE composition (e.g., a subject LINE pharmaceutical composition, a subject LINE immunogenic composition). In some embodiments, the present invention provides methods for treating cancer in an individual, the methods generally involving administering to an individual in need thereof an effective amount of a subject LINE immunogenic composition, e.g., a subject immunogenic composition comprising a subject LINE polypeptide or a subject LINE polynucleotide. The present invention provides use of a subject LINE polypeptide, a subject LINE polynucleotide, or a subject LINE composition in the preparation of a medicament for the treatment of a cancer in an individual. The present invention provides use of a subject LINE immunogenic composition (e.g., a subject immunogenic composition comprising a subject LINE polypeptide or a subject LINE polynucleotide) in the preparation of a medicament for the treatment of a cancer in an individual. The present invention provides a subject LINE polypeptide, a subject LINE polynucleotide, or a subject LINE composition for treating a cancer in an individual. The present invention provides a subject LINE immunogenic composition (e.g., a subject immunogenic composition comprising a subject LINE polypeptide or a subject LINE polynucleotide) for treating a cancer in an individual.

For example, an effective amount of a subject LINE polypeptide, a subject LINE polynucleotide, or a subject LINE composition is administered to an individual having a tumor (e.g., a solid tumor), wherein the cells of the tumor express a LINE polypeptide, e.g. a LINE-1 polypeptide, as a marker of the cancerous state.

For example, an effective amount of a subject immunogenic composition comprising one or more LINE polypeptides is administered to an individual having a tumor (e.g., a solid tumor), wherein the cells of the tumor express a LINE polypeptide, e.g. a LINE-1 polypeptide, as a marker of the cancerous state.

As another example, an effective amount of a subject LINE polypeptide, a subject LINE polynucleotide, or a subject LINE composition is administered to a subject having a tumor, wherein the tissue from which the tumor expresses a LINE polypeptide (e.g., a LINE-1 polypeptide) in the non-cancerous state and such tissue exhibits an increase (e.g., an at least about 15%, at least about 25%, at least about 30%, at least about 40%, at least about 50%, at least about 75%, at least about 2-fold, at least about 5-fold, or at least about 10-fold, or more than 10-fold, increase) in expression of the LINE polypeptide as a marker of the cancerous state.

As another example, an effective amount of a subject immunogenic composition is administered to a subject having a tumor, wherein the tissue from which the tumor expresses a LINE polypeptide (e.g., a LINE-1 polypeptide) in the non-cancerous state and such tissue exhibits an increase (e.g., an at least about 15%, at least about 25%, at least about 30%, at least about 40%, at least about 50%, at least about 75%, at least about 2-fold, at least about 5-fold, or at least about 10-fold, or more than 10-fold, increase) in expression of the LINE polypeptide as a marker of the cancerous state.

Cancers amenable to treatment with subject immunogenic compositions include ovarian cancer, breast cancer, melanoma, prostate cancer, and testicular cancer (including seminoma, teratoma, and embryonal carcinoma).

In some embodiments, in the context of cancer treatment, an “effective amount” of a subject LINE polypeptide, a subject LINE polynucleotide, or a subject LINE composition is an amount that, when administered to an individual in one or more doses, reduces one or more of tumor size, cancer cell number, and cancer cell metastasis by at least about 10%, at least about 20%, at least about 30%, at least about 40%, at least about 50%, at least about 60%, at least about 70%, at least about 80%, or at least about 90%, up to total eradication of the cancer.

In some embodiments, in the context of cancer treatment, an “effective amount” of a subject immunogenic composition is an amount that, when administered to an individual in one or more doses, reduces one or more of tumor size, cancer cell number, and cancer cell metastasis by at least about 10%, at least about 20%, at least about 30%, at least about 40%, at least about 50%, at least about 60%, at least about 70%, at least about 80%, or at least about 90%, up to total eradication of the cancer.

In some embodiments, an “effective amount” of a subject LINE polypeptide, a subject LINE polynucleotide, or a subject LINE composition is an amount that, when administered to an individual in one or more doses, results in an increase in the number of T cells specific for an epitope present on a cancer cell. In some embodiments, an “effective amount” of a subject LINE polypeptide, a subject LINE polynucleotide, or a subject LINE composition is an amount that, when administered to an individual in one or more doses, results in an increase of at least about 25%, at least about 50%, at least about 100% or 2-fold, at least about 5-fold, at least about 10-fold, or at least about 100-fold, or more, in the number of T cells specific for an epitope present on a cancer cell, compared with the number of T cells specific for a cancer cell epitope in the individual before treatment with the subject LINE polypeptide, subject LINE polynucleotide, or the subject LINE composition.

In some embodiments, an “effective amount” of a subject immunogenic composition is an amount that, when administered to an individual in one or more doses, results in an increase in the number of T cells specific for an epitope present on a cancer cell. In some embodiments, an “effective amount” of a subject immunogenic composition is an amount that, when administered to an individual in one or more doses, results in an increase of at least about 25%, at least about 50%, at least about 100% or 2-fold, at least about 5-fold, at least about 10-fold, or at least about 100-fold, or more, in the number of T cells specific for an epitope present on a cancer cell, compared with the number of T cells specific for a cancer cell epitope in the individual before treatment with the immunogenic composition.

In some embodiments, an “effective amount” of a subject LINE polypeptide, a subject LINE polynucleotide, or a subject LINE composition is an amount that, when administered to an individual in one or more doses, results in an increase in the number of CD8⁺ T cells specific for an epitope present on a cancer cell. In some embodiments, an “effective amount” of a subject LINE polypeptide, a subject LINE polynucleotide, or a subject LINE composition is an amount that, when administered to an individual in one or more doses, results in an increase of at least about 25%, at least about 50%, at least about 100% or 2-fold, at least about 5-fold, at least about 10-fold, or at least about 100-fold, or more, in the number of CD8⁺ T cells specific for a an epitope present on a cancer cell, compared with the number of CD8⁺ T cells specific for a cancer cell epitope in the individual before treatment with the subject LINE polypeptide, the subject LINE polynucleotide, or the subject LINE composition.

In some embodiments, an “effective amount” of a subject immunogenic composition is an amount that, when administered to an individual in one or more doses, results in an increase in the number of CD8⁺ T cells specific for an epitope present on a cancer cell. In some embodiments, an “effective amount” of a subject immunogenic composition is an amount that, when administered to an individual in one or more doses, results in an increase of at least about 25%, at least about 50%, at least about 100% or 2-fold, at least about 5-fold, at least about 10-fold, or at least about 100-fold, or more, in the number of CD8⁺ T cells specific for a an epitope present on a cancer cell, compared with the number of CD8⁺ T cells specific for a cancer cell epitope in the individual before treatment with the immunogenic composition.

In some embodiments, a subject LINE polypeptide, a subject LINE polynucleotide, or a subject LINE composition (e.g., a subject LINE immunogenic composition) is administered to an individual in need thereof as an adjuvant therapy to a standard cancer therapy. Standard cancer therapies include surgery (e.g., surgical removal of cancerous tissue), radiation therapy, bone marrow transplantation, chemotherapeutic treatment, biological response modifier treatment, and certain combinations of the foregoing.

Radiation therapy includes, but is not limited to, x-rays or gamma rays that are delivered from either an externally applied source such as a beam, or by implantation of small radioactive sources.

Chemotherapeutic agents are non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones.

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation; therefore, compounds that bind to the estrogen receptor are used to block this activity. Corticosteroids may inhibit T cell proliferation.

Other chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include immunosuppressants, e.g. mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

“Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

Biological response modifiers suitable for use in connection with the methods of the invention include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) IFN-α; (7) IFN-γ (8) colony-stimulating factors; and (9) inhibitors of angiogenesis.

### Methods for Treating Autoimmune Disorders

The present invention provides methods of treating an autoimmune disorder in an individual, the methods generally involving administering to an individual in need thereof a subject LINE polypeptide (e.g. a subject isolated LINE polypeptide, a subject synthetic LINE polypeptide), a subject LINE polynucleotide, or a subject LINE composition (e.g., a subject LINE pharmaceutical composition, a subject LINE immunogenic composition) in an amount effective to reduce a subject's immune response to a LINE polypeptide, thereby treating the autoimmune disease. Autoimmune disorders that can be treated with a subject method include, but are not limited to, multiple sclerosis, rheumatoid arthritis, systemic lupus erythematosus, and Type 1 diabetes.

The present invention provides methods for treating an autoimmune disorder in an individual, the methods generally involving administering to an individual in need thereof an effective amount of a subject LINE polypeptide (e.g. a subject isolated LINE polypeptide, a subject synthetic LINE polypeptide), a subject LINE polynucleotide, or a subject LINE composition (e.g., a subject LINE pharmaceutical composition, a subject LINE immunogenic composition). In some embodiments, the present invention provides methods for treating an autoimmune disorder in an individual, the methods generally involving administering to an individual in need thereof an effective amount of a subject LINE immunogenic composition, e.g., a subject immunogenic composition comprising a subject LINE polypeptide or a subject LINE polynucleotide. The present invention provides use of a subject LINE polypeptide, a subject LINE polynucleotide, or a subject LINE composition in the preparation of a medicament for the treatment of an autoimmune disorder in an individual. The present invention provides use of a subject LINE immunogenic composition (e.g., a subject immunogenic composition comprising a subject LINE polypeptide or a subject LINE polynucleotide) in the preparation of a medicament for the treatment of an autoimmune disorder in an individual. The present invention provides a subject LINE polypeptide, a subject LINE polynucleotide, or a subject LINE composition for treating an autoimmune disorder in an individual. The present invention provides a subject LINE immunogenic composition (e.g., a subject immunogenic composition comprising a subject LINE polypeptide or a subject LINE polynucleotide) for treating an autoimmune disorder in an individual.

In some embodiments, an effective amount of a subject LINE polypeptide, a subject LINE polynucleotide, or a subject LINE composition is an amount that is effective to reduce a subject's immune response to a LINE polypeptide by at least about 10%, at least about 15%, at least about 20%, at least about 25%, at least about 30%, at least about 35%, at least about 40%, at least about 45%, at least about 50%, or more than 50%, compared to the level of the subject's immune response to the LINE polypeptide in absence of treatment with a subject LINE polypeptide.

In some embodiments, a subject method is effective in reducing autoreactivity, where “reducing autoreactivity” includes one or more of reducing the number of autoreactive cells; reducing the activity of an autoreactive cell; and reducing the level of autoreactive antibody. Autoreactivity depends on the interactions of a number of white blood cells, including but not limited to, T lymphocytes, B cells, natural killer (NK) cells and dendritic cells. T lymphocytes include CD4⁺ T lymphocytes and CD8⁺ lymphocytes. B cells can function both as antigen presenting cells and producers of autoantibodies that can target tissues. In some embodiments, the subject method can alter the activities or numbers of these cells involved in various autoimmune reactivities. In some embodiments, a subject method is effective to reduce the number and/or activity of an autoreactive cell in an individual by at least about 5%, at least about 10%, at least about 25%, at least about 30%, at least about 40%, at least about 50%, at least about 60%, at least about 70%, at least about 80%, or at least about 90%, or more, when compared to the number and/or level of autoreactive cells in the individual not treated with a subject LINE polypeptide, a subject LINE polynucleotide, or a subject LINE composition.

In some embodiments, a subject method is effective to reduce the number and/or activity of an autoreactive T lymphocyte. Thus, in some embodiments, an effective amount of a subject LINE polypeptide, a subject LINE polynucleotide, or a subject LINE composition is an amount that is effective to reduce the number and/or activity of autoreactive T lymphocytes in an individual by at least about 5%, at least about 10%, at least about 25%, at least about 30%, at least about 40%, at least about 50%, at least about 60%, at least about 70%, at least about 80%, or at least about 90%, or more, when compared to the number and/or level of autoreactive T lymphocytes in the individual not treated with the subject LINE polypeptide, the subject LINE polynucleotide, or the subject LINE composition.

In some embodiments, a subject method is effective to reduce the number and/or activity of an autoreactive B cell. Thus, in some embodiments, an effective amount of a subject LINE polypeptide, a subject LINE polynucleotide, or a subject LINE composition is an amount that is effective to reduce the number and/or activity of autoreactive B cells in an individual by at least about 5%, at least about 10%, at least about 25%, at least about 30%, at least about 40%, at least about 50%, at least about 60%, at least about 70%, at least about 80%, or at least about 90%, or more, when compared to the number and/or level of autoreactive B cells in the individual not treated with the subject LINE polypeptide, the subject LINE polynucleotide, or the subject LINE composition.

Activities of an autoreactive T lymphocyte include, but are not limited to, cytolytic activity toward a “self” cell; secretion of cytokine(s); secretion of chemokine(s); responsiveness to chemokine(s); and trafficking. In some embodiments, an effective amount of a subject LINE polypeptide, a subject LINE polynucleotide, or a subject LINE composition is an amount that is effective to reduce one or more activities of an autoreactive T lymphocyte in an individual.

Whether administration of a subject LINE polypeptide, a subject LINE polynucleotide, or a subject LINE composition is effective to reduce the number and/or activity of an autoreactive T lymphocyte in an individual is readily determined using known assays. For example, where the autoreactive T lymphocytes are specific for an autoantigen, the number and activity level of autoantigen-specific T lymphocytes is determined using, e.g., a mixed lymphocyte reaction in which irradiated cells comprising a detectable label in the cytoplasm and displaying the autoantigen are mixed with lymphocytes from the individual. Release of detectable label from the cytoplasm of the autoantigen-displaying cells indicates the presence in the individual of autoreactive lymphocytes. Methods of detecting autoreactive T lymphocytes associated with Type 1 diabetes are known in the art; and any such methods can be used. See, e.g., U.S. Pat. No. 6,022,697 for a discussion of a method of detecting autoreactive T lymphocytes associated with Type 1 diabetes.

In some embodiments, an effective amount of a subject LINE polypeptide, a subject LINE polynucleotide, or a subject LINE composition is an amount that is effective to reduce the severity of one or more symptoms of an autoimmune disease. For example, in some embodiments, an effective amount of a subject LINE polypeptide, a subject LINE polynucleotide, or a subject LINE composition is an amount that is effective to reduce the severity of one or more symptoms of an autoimmune disease by at least about 5%, at least about 10%, at least about 25%, at least about 30%, at least about 40%, at least about 50%, at least about 60%, at least about 70%, at least about 80%, or at least about 90%, or more, when compared to the severity of the symptom in an individual not treated with the subject LINE polypeptide, the subject LINE polynucleotide, or the subject LINE composition.

Symptoms associated with autoimmune disorders are known in the art. See, e.g., “Textbook of the Autoimmune Diseases” R. G. Lahita, Ed. (2000) Lippincott Williams & Wilkins, 1^{st} ed. The following are non-limiting examples.

Multiple sclerosis is characterized by various symptoms and signs of central nervous system (CNS) dysfunction, with remissions and recurring exacerbations. The most common presenting symptoms are paresthesias in one or more extremities, in the trunk, or on one side of the face; weakness or clumsiness of a leg or hand; or visual disturbances, e.g. partial blindness and pain in one eye (retrobulbar optic neuritis), dimness of vision, or scotomas. Other common early symptoms are ocular palsy resulting in double vision (diplopia), transient weakness of one or more extremities, slight stiffness or unusual fatigability of a limb, minor gait disturbances, difficulty with bladder control, vertigo, and mild emotional disturbances.

Diabetes Mellitus (DM) is syndrome characterized by hyperglycemia resulting from absolute or relative impairment in insulin secretion and/or insulin action. Although it may occur at any age, type I DM most commonly develops in childhood or adolescence and is the predominant type of DM diagnosed before age 30. This type of diabetes accounts for 10 to 15% of all cases of DM and is characterized clinically by hyperglycemia.

### Combination Therapies

In some embodiments, a subject treatment method will involve administering to an individual in need thereof an effective amount of a subject LINE polypeptide, a subject LINE polynucleotide, or a subject LINE composition; and at least one additional agent that is effective for the treatment of an autoimmune disorder. In some embodiments, the at least one additional agent is other than a subject LINE polypeptide, a subject LINE polynucleotide, or a subject LINE composition.

In some embodiments, a subject treatment method will involve administering to an individual in need thereof an effective amount of a subject LINE polypeptide; and at least one additional agent that is effective for the treatment of an autoimmune disorder. In some embodiments, the at least one additional agent is other than a subject LINE polypeptide.

Those skilled in the art are aware of agents (other than a subject LINE polypeptide) that are suitable for treating autoimmune disorders. For example, agents that are suitable for treating Type 1 diabetes include insulin, including naturally occurring insulin, insulin analogs, and the like.

Insulin that is suitable for use herein includes, but is not limited to, regular insulin, semilente, NPH, lente, protamine zinc insulin (PZI), ultralente, insuline glargine, insulin aspart, acylated insulin, monomeric insulin, superactive insulin, hepatoselective insulin, and any other insulin analog or derivative, and mixtures of any of the foregoing. Insulin that is suitable for use herein includes, but is not limited to, the insulin forms disclosed in U.S. Pat. Nos. 4,992,417; 4,992,418; 5,474,978; 5,514,646; 5,504,188; 5,547,929; 5,650,486; 5,693,609; 5,700,662; 5,747,642; 5,922,675; 5,952,297; and 6,034,054; and published PCT applications WO 00/121197; WO 09/010,645; and WO 90/12814. Insulin analogs include, but are not limited to, superactive insulin analogs, monomeric insulins, and hepatospecific insulin analogs.

### Methods of Treating Schizophrenia

The present invention provides methods for treating schizophrenia in an individual, the methods generally involving administering to an individual in need thereof an effective amount of a subject LINE polypeptide (e.g. a subject isolated LINE polypeptide, a subject synthetic LINE polypeptide), a subject LINE polynucleotide, or a subject LINE composition (e.g., a subject LINE pharmaceutical composition, a subject LINE immunogenic composition). The present invention provides use of a subject LINE polypeptide, a subject LINE polynucleotide, or a subject LINE composition in the preparation of a medicament for the treatment of schizophrenia in an individual. The present invention provides a subject LINE polypeptide, a subject LINE polynucleotide, or a subject LINE composition for treating schizophrenia in an individual.

In these embodiments, an “effective amount” of a subject LINE polypeptide, a subject LINE polynucleotide, or a subject LINE composition is an amount that, when administering to an individual in need thereof in one or more doses, reduces at least one symptom of schizophrenia by at least about 10%, at least about 15%, at least about 20%, at least about 25%, at least about 30%, at least about 35%, at least about 40%, at least about 45%, at least about 50%, or more, compared to the level or severity of the symptom in the individual in the absence of treatment with the subject LINE polypeptide, the subject LINE polynucleotide, or the subject LINE composition. Symptoms of schizophrenia are known in the art, and include, e.g., “positive” symptoms (e.g., delusions, hallucinations, disorganized speech, grossly disorganized or catatonic behavior); and “negative” symptoms (e.g, alogia, affective flattening, avolition).

### Formulations

A subject LINE polypeptide, or a subject LINE polynucleotide, as described above, can be formulated in any of a variety of ways for administration to an individual in need thereof. The present invention provides pharmaceutical formulations comprising a LINE polypeptide or a subject LINE polynucleotide. Immunogenic compositions comprising a LINE polypeptide or a subject LINE polynucleotide are described above. Additional formulations are described below.

A subject formulation comprising a LINE polypeptide or a subject LINE polynucleotide generally includes one or more of an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylprrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinylpyrrolidone or aluminum stearate), a dispersing agent (e.g., hydroxypropylmethylcellulose), a diluent (e.g., water), and base wax (e.g., cocoa butter, white petrolatum or polyethylene glycol).

Tablets comprising an active agent may be coated with a suitable film-forming agent, e.g., hydroxypropylmethyl cellulose, hydroxypropyl cellulose or ethyl cellulose, to which a suitable excipient may optionally be added, e.g., a softener such as glycerol, propylene glycol, diethylphthalate, or glycerol triacetate; a filler such as sucrose, sorbitol, xylitol, glucose, or lactose; a colorant such as titanium hydroxide; and the like.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the agent adequate to achieve the desired state in the subject being treated. The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, e.g., for use in inducing or enhancing an immune response to a lentivirus, a LINE polypeptide or a subject LINE polynucleotide is formulated for vaginal delivery. A subject formulation for intravaginal administration is formulated as an intravaginal bioadhesive tablet, intravaginal bioadhesive microparticle, intravaginal cream, intravaginal lotion, intravaginal foam, intravaginal ointment, intravaginal paste, intravaginal solution, or intravaginal gel.

### Dosages

The appropriate dosage of a subject LINE polypeptide or a subject LINE polynucleotide is a dosage that, when administered in one or multiple doses, has the desired effect (e.g., increases a T cell immune response to a lentivirus; increases an immune response to a cancer cell; reduces an autoimmune response; etc.), will vary, depending on various factors, but will generally be in the range of from about 1 μg to about 100 mg, e.g., from about 1 μg to about 5 μg, from about 5 μg to about 10 μg, from about 10 μg to about 25 μg, from about 25 μg to about 50 μg, from about 50 μg to about 100 μg, from about 100 μg to about 500 μg, from about 500 μg to about 1 mg, from about 1 mg to about 10 mg, from about 10 mg to about 50 mg, or from about 50 mg to about 100 mg, administered in one dose or divided into multiple doses.

In some embodiments, the amount of a subject LINE polypeptide per dose is determined on a per body weight basis. For example, in some embodiments, a subject LINE polypeptide is administered in an amount of from about 0.5 mg/kg to about 100 mg/kg, e.g., from about 0.5 mg/kg to about 1 mg/kg, from about 1 mg/kg to about 2 mg/kg, from about 2 mg/kg to about 3 mg/kg, from about 3 mg/kg to about 5 mg/kg, from about 5 mg/kg to about 7 mg/kg, from about 7 mg/kg to about 10 mg/kg, from about 10 mg/kg to about 15 mg/kg, from about 15 mg/kg to about 20 mg/kg, from about 20 mg/kg to about 25 mg/kg, from about 25 mg/kg to about 30 mg/kg, from about 30 mg/kg to about 40 mg/kg, from about 40 mg/kg to about 50 mg/kg per dose, from about 50 mg/kg to about 60 mg/kg, from about 60 mg/kg to about 70 mg/kg, from about 70 mg/kg to about 80 mg/kg, from about 80 mg/kg to about 90 mg/kg, or from about 90 mg/kg to about 100 mg/kg, or more than about 100 mg/kg.

In some embodiments, the amount of a subject LINE polynucleotide per dose is determined on a per body weight basis. For example, in some embodiments, a subject LINE polynucleotide is administered in an amount of from about 0.5 mg/kg to about 100 mg/kg, e.g., from about 0.5 mg/kg to about 1 mg/kg, from about 1 mg/kg to about 2 mg/kg, from about 2 mg/kg to about 3 mg/kg, from about 3 mg/kg to about 5 mg/kg, from about 5 mg/kg to about 7 mg/kg, from about 7 mg/kg to about 10 mg/kg, from about 10 mg/kg to about 15 mg/kg, from about 15 mg/kg to about 20 mg/kg, from about 20 mg/kg to about 25 mg/kg, from about 25 mg/kg to about 30 mg/kg, from about 30 mg/kg to about 40 mg/kg, from about 40 mg/kg to about 50 mg/kg per dose, from about 50 mg/kg to about 60 mg/kg, from about 60 mg/kg to about 70 mg/kg, from about 70 mg/kg to about 80 mg/kg, from about 80 mg/kg to about 90 mg/kg, or from about 90 mg/kg to about 100 mg/kg, or more than about 100 mg/kg.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

In some embodiments, multiple doses of a subject LINE polypeptide or a subject LINE polynucleotide are administered. The frequency of administration of a LINE polypeptide or a subject LINE polynucleotide can vary depending on any of a variety of factors, e.g., severity of the symptoms, etc. For example, in some embodiments, a LINE polypeptide or a subject LINE polynucleotide is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The duration of administration of a LINE polypeptide, e.g., the period of time over which a LINE polypeptide is administered, can vary, depending on any of a variety of factors, e.g., patient response, etc. For example, a LINE polypeptide can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

The duration of administration of a LINE polynucleotide, e.g., the period of time over which a LINE polynucleotide is administered, can vary, depending on any of a variety of factors, e.g., patient response, etc. For example, a LINE polynucleotide can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

### Routes of Administration

Conventional and pharmaceutically acceptable routes of administration include intranasal, intramuscular, intratracheal, intratumoral, transdermal, subcutaneous, intradermal, topical application, intravenous, vaginal, nasal, and other parenteral routes of administration. Suitable routes of administration also include oral and rectal routes. Routes of administration may be combined, if desired, or adjusted depending upon the agent and/or the desired effect. The composition can be administered in a single dose or in multiple doses.

A subject LINE polypeptide, a subject LINE polynucleotide, or a subject LINE composition can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, vaginal, transdermal, subcutaneous, intramuscular, intraorbital, intracapsular, intraspinal, intrasternal, intratumoral, peritumoral, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

A subject LINE polypeptide, a subject LINE polynucleotide, or a subject LINE composition can also be delivered to the subject by enteral administration. Enteral routes of administration include, but are not necessarily limited to, oral and rectal (e.g., using a suppository) delivery.

A subject LINE polypeptide, a subject LINE polynucleotide, or a subject LINE composition (e.g., a subject LINE pharmaceutical composition, a subject LINE immunogenic composition) can be delivered to mucosal tissue, e.g., to vaginal tissue, to rectal tissue, etc.

### Methods of Generating LINE-Specific CTLs

The present invention provides methods of generating a population of LINE-specific CD8⁺ T cells in vitro. The methods generally involve contacting a CD8⁺ T cell, or a precursor thereof, with a LINE polypeptide in association with an antigen-presenting platform, where the contacting is performed in vitro. The methods are useful for generating a population of LINE polypeptide-specific CD8⁺ T cells, which are in turn useful in methods of treating disorders such as retrovirus infection, lentivirus infection (e.g., HIV infection), and cancer.

In some embodiments, CD8⁺ T cells are obtained from an individual, and are contacted in vitro with a LINE polypeptide in association with an antigen-presenting platform. In some embodiments, a mixed population of cells that comprises CD8⁺ T cells is obtained from an individual; and CD8⁺ T cells are isolated from the mixed population, generating an unstimulated CD8⁺ T cell population. The unstimulated CD8⁺ T cell population is then contacted in vitro to a LINE polypeptide in association with an antigen-presenting platform. The contacting step activates at least a portion of the unstimulated CD8⁺ T cell population having T cell receptors capable of binding a LINE polypeptide to become specific for a LINE polypeptide.

The source of the mixed cell population that comprises a CD8⁺ T cell can be, e.g., whole blood. The mixed cell population can manipulated in one or more ways or steps, e.g., to remove red blood cells; to select for CD8⁺ T cells; and/or to select against CD4⁺ T cells or other non-CD8⁺ cell populations. The number of unstimulated CD8⁺ cells can range from about 10² to about 10⁹ cells, e.g., from about 10² cells to about 10³ cells, from about 10³ cells to about 10⁴ cells, from about 10⁴ cells to about 10⁵ cells, from about 10⁹ cells to about 5×10⁵ cells, from about 5×10⁵ cells to about 10⁶ cells, from about 10⁶ cells to about 5×10⁶ cells, from about 5×10⁶ cells to about 10⁷ cells, from about 10⁷ cells to about 5×10⁷ cells, from about 5×10⁷ cells to about 10⁸ cells, from about 10⁸ cells to about 5×10⁸ cells, or from about 5×10⁸ cells to about 10⁹ cells.

The antigen-presenting platform can be an antigen-presenting cell (APC), e.g., an APC pulsed with a LINE polypeptide, where the APC can be live or can be inactivated. In some embodiments, the antigen-presenting platform is a bead (e.g., a plastic bead, a magnetic bead, etc.), or other particle, to which a LINE polypeptide is bound. Antigen-presenting platforms other than naturally-occurring APCs are known in the art and include, but are not limited to, beads; inactivated surface-engineered viruses (see, e.g., Mosca et al. (2007) Retrovirol. 4:32); artificial APCs, e.g., liposomes (see, e.g, U.S. Patent Publication No. 2006/0034865); and the like.

The antigen-presenting platform will include, in addition to a LINE polypeptide, one or more surface molecules sufficient for stimulating expansion of a LINE-specific CD8⁺ T cell population, e.g., MHC class I molecules (e.g., HLA Class I molecules), etc. The antigen-presenting platform can also include one or more co-stimulatory molecules, where suitable co-stimulatory molecules include, but are not limited to, an anti-CD28 antibody, an anti-CD49d antibody, and the like).

The unstimulated CD8⁺ T cells are contacted in vitro with a LINE polypeptide in association with an antigen-presenting platform; and the number of LINE-specific CD8⁺ T cells is increased. The method results in a 10-fold to a 10⁶-fold increase in the number of LINE-specific CD8⁺ T cells. The number of LINE-specific CD8⁺ cells obtained by a subject method can range from about 10³ to about 10⁹ cells, e.g., from about 10³ cells to about 10⁴ cells, from about 10⁴ cells to about 10⁵ cells, from about 10⁵ cells to about 5×10⁵ cells, from about 5×10⁵ cells to about 10⁶ cells, from about 10⁶ cells to about 5×10⁶ cells, from about 5×10⁶ cells to about 10⁷ cells, from about 10⁷ cells to about 5×10⁷ cells, from about 5×10⁷ cells to about 10⁸ cells, from about 10⁸ cells to about 5×10⁸ cells, or from about 5×10⁸ cells to about 10⁹ cells.

The present disclosure provides treatment methods using the LINE-specific CD8⁺ T cells. In some embodiments, the methods are methods of treating an HIV infection. In other embodiments, the methods are methods of treating cancer. The methods generally involve administering to an individual in need thereof an effective amount of LINE-specific CD8⁺ T cells. In some embodiments, the LINE-specific CD8⁺ T cells are autologous, e.g., the LINE-specific CD8⁺ T cells are administered to the same individual from which the mixed cell population was obtained (i.e., the donor individual and the recipient individual are the same). In other embodiments, the LINE-specific CD8⁺ T cells are allogeneic, e.g., the LINE-specific CD8⁺ T cells are administered to an individual (a recipient individual) not genetically identical to the individual from which the mixed cell population was obtained (the donor individual).

In some embodiments, the LINE-specific CD8⁺ T cells are administered to a recipient individual in an amount of from about 10³ to about 10⁹ cells, e.g., from about 10³ cells to about 10⁴ cells, from about 10⁴ cells to about 10⁵ cells, from about 10⁵ cells to about 5×10⁵ cells, from about 5×10⁵ cells to about 10⁶ cells, from about 10⁶ cells to about 5×10⁶ cells, from about 5×10⁶ cells to about 10⁷ cells, from about 10⁷ cells to about 5×10⁷ cells, from about 5×10⁷ cells to about 10⁸ cells, from about 10⁸ cells to about 5×10⁸ cells, or from about 5×10⁸ cells to about 10⁹ cells, in one or more doses.

### Diagnostic Methods

The present invention provides various diagnostic methods, which methods utilize a subject LINE polypeptide or a subject LINE composition. Subject diagnostic methods include methods for monitoring a patient's response to treatment; methods for staging a disease; and methods for detecting a disease.

In some embodiments, a subject diagnostic method involves detecting the presence in an individual of a cancer cell that produces a LINE polypeptide. Methods for detecting a cancer cell that produces a LINE polypeptide include immunological methods, e.g., use of an antibody specific for a LINE polypeptide, where immunological assays include, e.g., immunohistological assays, and fluorescence activated cell analysis assays (e.g., fluorescence activated cell sorting assays, using a fluorescently labeled antibody to a LINE polypeptide).

In other embodiments, a subject diagnostic method generally involves detecting the number of LINE-specific CD8⁺ T cells in a biological sample obtained from an individual. The number of LINE-specific CD8⁺ T cells can be determined using, e.g., a ⁵¹Cr release assay, where target cells pulsed with a LINE peptide and labeled with ⁵¹Cr are contacted with a test sample that may contain LINE-specific CD8⁺ T cells. The number of LINE-specific CD8⁺ T cells is determined by measuring release of ⁵¹Cr from the target cells.

In other embodiments, a subject diagnostic method involves detecting a LINE polypeptide in the serum or plasma (or other biological fluid) of an individual. Detection of a LINE polypeptide in a biological fluid obtained from an individual can be carried out using, e.g., immunological assays employing antibody specific for a LINE polypeptide. Suitable immunological assays include, but are not limited to, enzyme-linked immunosorbent assays (ELISA), radioimmunoassays (RIA), protein blot (“Western blot”) assays, immunoprecipitation assays, and the like.

### LINE-Specific Antibodies

As noted above, in some embodiments, a subject diagnostic assay will employ an antibody specific for a LINE polypeptide (an “anti-LINE antibody”). Suitable anti-LINE antibodies include whole antibody of any isotype; epitope-binding fragments of an anti-LINE antibody; polyclonal antibodies; monoclonal antibodies; artificial antibodies; single-chain antibodies; and the like. In some embodiments, the present invention provides antibodies that bind specifically to a subject LINE polypeptide. A subject LINE polypeptide-specific antibody can be used in a subject diagnostic assay. In some embodiments, a subject antibody is isolated. In some embodiments, a subject antibody is artificial or synthetic.

Monoclonal antibodies are produced by conventional techniques. Generally, the spleen and/or lymph nodes of an immunized host animal provide a source of plasma cells. The plasma cells are immortalized by fusion with myeloma cells to produce hybridoma cells. Culture supernatant from individual hybridomas is screened using standard techniques to identify those producing antibodies with the desired specificity. Suitable animals for production of monoclonal antibodies include mouse, rat, hamster, guinea pig, rabbit, etc. The antibody may be purified from the hybridoma cell supernatants or ascites fluid by conventional techniques, e.g. affinity chromatography using protein bound to an insoluble support, protein A sepharose, etc.

The antibody may be produced as a single chain, instead of the normal multimeric structure. Single chain antibodies are described in Jost et al. (1994) J. B. C. 269:26267-73, and others. DNA sequences encoding the variable region of the heavy chain and the variable region of the light chain are ligated to a spacer encoding at least about 4 amino acids of small neutral amino acids, including glycine and/or serine. The protein encoded by this fusion allows assembly of a functional variable region that retains the specificity and affinity of the original antibody.

Suitable anti-LINE antibodies also include “artificial” antibodies, e.g., antibodies and antibody fragments produced and selected in vitro. In some embodiments, such antibodies are displayed on the surface of a bacteriophage or other viral particle. In many embodiments, such artificial antibodies are present as fusion proteins with a viral or bacteriophage structural protein, including, but not limited to, M13 gene III protein. Methods of producing such artificial antibodies are well known in the art. See, e.g., U.S. Pat. Nos. 5,516,637; 5,223,409; 5,658,727; 5,667,988; 5,498,538; 5,403,484; 5,571,698; and 5,625,033.

Antibody fragments, such as Fv, F(ab′)₂ and Fab may be prepared by cleavage of the intact protein, e.g. by protease or chemical cleavage. Alternatively, a truncated gene is designed. For example, a chimeric gene encoding a portion of the F(ab′)₂ fragment would include DNA sequences encoding the CH1 domain and hinge region of the H chain, followed by a translational stop codon to yield the truncated molecule.

An anti-LINE antibody will in some embodiments be detectably labeled, e.g., with a radioisotope, an enzyme which generates a detectable product, a fluorescent protein, a chromogenic protein, and the like. An anti-LINE antibody may be further conjugated to other moieties, such as members of specific binding pairs, e.g., biotin (member of biotin-avidin specific binding pair), and the like. An anti-LINE antibody may also be bound to a solid support, including, but not limited to, polystyrene plates or beads, magnetic beads, test strips, membranes, and the like.

An antibody specific for a LINE polypeptide can be labeled, directly or indirectly. Direct labels include radioisotopes (e.g., ¹²⁵I; ³⁵S, and the like); enzymes whose products are detectable (e.g., luciferase, (β-galactosidase, horse radish peroxidase, alkaline phosphatase, and the like); fluorescent labels (e.g., fluorescein isothiocyanate, rhodamine, phycoerythrin, and the like); fluorescence emitting metals, e.g., ¹⁵²Eu, or others of the lanthanide series, attached to the antibody through metal chelating groups such as EDTA; chemiluminescent compounds, e.g., luminol, isoluminol, acridinium salts, and the like; bioluminescent compounds, e.g., luciferin; fluorescent proteins (e.g., a green fluorescent protein, a yellow fluorescent protein, etc.); and the like. Indirect labels include second antibodies specific for LINE-specific antibodies, wherein the second antibody is labeled as described above; and members of specific binding pairs, e.g., biotin-avidin, and the like.

In some embodiments, an anti-LINE antibody comprises, covalently linked to the antibody, a protein that provides for a detectable signal. Suitable proteins include, but are not limited to, fluorescent proteins and enzymes (e.g., β-galactosidase, luciferase, horse radish peroxidase, alkaline phosphatase, etc.). Suitable fluorescent proteins include, but are not limited to, a green fluorescent protein (GFP), including, but not limited to, a GFP derived from Aequoria victoria or a derivative thereof, a number of which are commercially available; a GFP from a species such as Renilla reniformis, Renilla mulleri, or Ptilosarcus guernyi, as described in, e.g., WO 99/49019 and Peelle et al. (2001) J. Protein Chem. 20:507-519; any of a variety of fluorescent and colored proteins from Anthozoan species, as described in, e.g., Matz et al. (1999) Nature Biotechnol. 17:969-973, U.S. Patent Publication No. 2002/0197676, or U.S. Patent Publication No. 2005/0032085; and the like.

In certain embodiments, a subject diagnostic assay employs an antibody specific for a LINE polypeptide, wherein the antibody specific for the LINE polypeptide specifically excludes antibodies, or binding fragments thereof, having binding affinity for LINE-1 p40, e.g., polyclonal antibody, AH40.1.

### Monitoring Patient Response to Treatment for a Retrovirus Infection

In some embodiments, a subject LINE polypeptide composition is useful for monitoring a patient's response to treatment for a retrovirus infection, e.g., an HIV infection, an HTLV infection, etc.

Thus, the present disclosure further provides methods for monitoring a patient's response to treatment for a retrovirus infection, e.g., an HIV infection. The methods generally involve contacting a white blood cell (WBC) from a patient in vitro with a subject LINE polypeptide; and detecting a cytokine secreted by the WBC in response to contact with the LINE polypeptide. A reduction in cytokine production by the WBC in response to contact with a LINE polypeptide, compared to the level of cytokine production by a WBC obtained from the individual before treatment or at an earlier time point during treatment, is an indication that the treatment is effective in treating the retrovirus infection (e.g., in achieving a reduction in viral load, in achieving an increase in CD4⁺ T lymphocyte levels (in the case of an HIV infection), and the like). Suitable WBCs include, but are not limited to, peripheral blood mononuclear cells (PBMC), isolated T lymphocytes, isolated CD4⁺ T lymphocytes, isolated CD8⁺ T lymphocytes, natural killer (NK) cells, natural killer T lymphocytes (NKT, e.g., NK1.1⁺ T lymphocytes), and the like.

For example, in some embodiments, a subject monitoring method comprises: a) contacting WBC in vitro with a subject synthetic LINE polypeptide, where the WBC is obtained from the patient at a first time point following the beginning of a treatment for a retroviral infection; and b) detecting a cytokine secreted by the WBC in response to contact with the LINE polypeptide, where a reduction in cytokine production by the WBC in response to contact with a LINE polypeptide, compared to the level of cytokine production by a control WBC in response to contact with the LINE polypeptide, indicates that the treatment is effective in treating the retrovirus infection, where the control WBC is obtained from the patient before the start of treatment, or at a time point during the treatment that is earlier than the first time point.

As another example, in some embodiments, a subject monitoring method comprises: a) contacting WBC in vitro with a subject synthetic LINE polypeptide, where the WBC is obtained from the patient at a second time point following the beginning of a treatment for a retroviral infection; and b) detecting a cytokine secreted by the WBC in response to contact with the LINE polypeptide, where a reduction in cytokine production by the WBC in response to contact with a LINE polypeptide, compared to the level of cytokine production by a control WBC in response to contact with the LINE polypeptide, indicates that the treatment is effective in treating the retrovirus infection, where the control WBC is obtained from the patient at a first time point following the beginning of treatment, where the first time point is earlier than the second time point.

In some embodiments, a subject LINE polypeptide composition is useful for monitoring a patient's response to treatment for an HTLV infection (e.g., an HTLV-I or HTLV-II infection). The methods generally involve contacting a white blood cell (WBC) from a patient in vitro with a subject LINE polypeptide; and detecting a cytokine secreted by the WBC in response to contact with the LINE polypeptide. A reduction in cytokine production by the WBC in response to contact with a LINE polypeptide is an indication that the treatment is effective in treating an HTLV-I or -II infection (e.g., in achieving a reduction in viral load, in achieving an increase in CD4⁺ T lymphocyte levels (in the case of an HIV infection), and the like). Suitable WBCs include, but are not limited to, peripheral blood mononuclear cells (PBMC), isolated T lymphocytes, isolated CD4⁺ T lymphocytes, isolated CD8⁺ T lymphocytes, natural killer (NK) cells, natural killer T lymphocytes (NKT, e.g., NK1.1⁺ T lymphocytes), and the like.

LINE polypeptides suitable for use in a subject monitoring method can be 6 amino acids, 7 amino acids, 8 amino acids, 9 amino acids, 10 amino acids, 11 amino acids, 12 amino acids, 12-15 amino acids, 15-18 amino acids, 18-20 amino acids, or 20-25 amino acids long, or longer. Suitable LINE polypeptides include any of the LINE polypeptides discussed above. In some embodiments, the LINE polypeptide comprises an amino acid sequence having at least about 85%, at least about 90%, at least about 95%, at least about 98%, at least about 99%, or 100%, amino acid sequence identity to an amino acid sequence as set forth in any one of SEQ ID NOs: 1-22.

Cytokines that are secreted from PBMCs and that are detected in a subject patient monitoring method include, but are not limited to, IFN-γ, TNF-α, and IL-2.

Methods for detecting secreted cytokines that are suitable for use in a subject patient monitoring method include, but are not limited to, immunological assays, e.g., enzyme-linked immunosorbent assay (ELISA), radioimmunoassay (RIA), an enzyme-linked immunospot (ELISPOT) assay; cellular assays; and the like.

In some embodiments, a reduction of at least about 10%, at least about 20%, at least about 30%, at least about 40%, at least about 50%, at least about 60%, at least about 70%, at least about 80%, or at least about 90% or more, in cytokine production by WBCs in response to contact with a LINE polypeptide, compared to the level of cytokine production by a WBC obtained from the patient before treatment or at an earlier time point during treatment, indicates that the treatment for the retrovirus infection is efficacious.

Patient samples comprising WBCs can be obtained before and after treatment; or at various times during the course of treatment, and the level of cytokine production compared between a sample taken at a first time point and a sample taken at a second (later) time point.

In some embodiments, PBMC obtained from a patient are contacted with one or more LINE polypeptides in vitro; and an ELISPOT assay is used to detect cytokine production. The ELISPOT assay has been described in the art. See, e.g., Lalvani et al. (1997) J. Exp. Med. 186:859; and U.S. Pat. No. 5,853,697. In these embodiments, the level of cytokines produced by the PBMC is expressed as the number of spot-forming units (SFU) per 10⁶ PBMC. A reduction in the number of SFU indicates that a treatment for a retrovirus infection is effective.

### Monitoring Patient Response to Cancer Treatment

In certain embodiments, methods of monitoring patient response to a treatment regimen for cancer are provided. For example, the level of a LINE polypeptide associated with the cancer is monitored, before, during a treatment regimen, and after a treatment regimen.

In some embodiments, the level of a LINE polypeptide is monitored, e.g., in serum, on the surface of a particular cell population, etc.

### Staging a Disease

The present disclosure provides methods of staging a disease in an individual, where the level of a LINE polypeptide is associated with the stage or severity of the disease. The methods generally involve detecting the level of a LINE polypeptide in a biological sample obtained from the individual. The level of the LINE polypeptide in the biological sample is correlated with the severity of the disease or disorder, and used to stage the disease.

In some embodiments, a subject method of staging a disease involves detecting the number of CD8⁺ T cells, in a biological sample obtained from an individual, that are specific for a subject LINE polypeptide. In some embodiments, the number of LINE-specific CD8⁺ T cells is an indication of the stage of the disease.

### Detecting a Disease

The present disclosure provides methods of detecting a disease such as a cancer in an individual, where the presence or level of a LINE polypeptide in a biological sample obtained from the individual indicates the presence of a cancerous cell in the biological sample (and hence the individual). The methods generally involve detecting the level of a LINE polypeptide in a biological sample obtained from the individual. Where the level of the LINE polypeptide is higher than the level associated with a normal cell, such is an indication of the presence in the sample of a cancerous cell.

In some embodiments, detecting a disease associated with abnormal LINE expression, e.g., abnormally increased LINE-1 expression in a cancerous tissue (e.g., a tumor) or HIV infected cell, involves detecting the number of CD8⁺ T cells in a biological sample obtained from an individual that are specific for a subject LINE polypeptide. Where a CD8⁺ T cell response for a subject LINE polypeptide is present in the biological sample, there is an indication that the individual is suffering from a disease associated with abnormal LINE expression.

### Subjects Suitable for Treatment

### Treatment of Retroviral Infection

The present disclosure contemplates methods which are suitable for treating individuals who have a retroviral infection, e.g., a lentiviral infection; uninfected individuals who are at risk of contracting a retroviral infection; individuals who were treated for a retroviral infection, but failed to respond to the treatment; and individuals who were treated for a retroviral infection, but who relapsed. In some embodiments, “at risk” individuals are individuals who are at greater risk than the general population for contracting a retroviral infection (e.g., a lentiviral infection such as an HIV infection).

In some embodiments, a subject immunogenic composition comprising a subject LINE polypeptide is administered to a naïve individual, e.g., an individual who is not infected with an HIV. Administration of a subject immunogenic composition to a naïve individual can reduce the severity of disease due to HIV infection and/or can limit the HIV infection and/or can clear the HIV infection, should the individual become infected with an HIV.

Administration of a subject immunogenic composition to a naïve individual can result in clearance of a sub-clinical infection with HIV, e.g., an HIV infection that does not result in development of symptoms of an HIV infection. For example, administration of a subject immunogenic composition to a naïve individual can result in clearance of a sub-clinical infection with HIV such that the individual does not develop clinical HIV infection (e.g., the individual does not seroconvert, does not develop detectable HIV viral load, does not have detectable levels of serum HIV antigen, etc.).

For example, the methods of the present invention are suitable for treating individuals who have a human immunodeficiency virus (HIV) infection; individuals who are naïve with respect to HIV infection, but who at risk of contracting an HIV infection; and individuals who were treated for an HIV infection, but who either failed to respond to the treatment, or who initially responded to treatment but subsequently relapsed. Such individuals include, but are not limited to, uninfected individuals with healthy, intact immune systems, but who are at risk for becoming HIV infected (“at-risk” individuals). At-risk individuals include, but are not limited to, individuals who have a greater likelihood than the general population of becoming HIV infected. Individuals at risk for becoming HIV infected include, but are not limited to, individuals at risk for HIV infection due to sexual activity with HIV-infected individuals; intravenous drug users; individuals who may have been exposed to HIV-infected blood, blood products, or other HIV-contaminated body fluids; and babies who are being nursed by HIV-infected mothers. Individuals suitable for treatment include individuals infected with, or at risk of becoming infected with HIV-1, HIV-2, or any variant thereof.

### Treatment of HTLV Infection

The above-described methods can be used to treat a human T cell leukemia virus (HTLV) infection in an individual, e.g., an HTLV-I or HTLV-II infection. Thus, a subject method is also suitable for treating individuals who have been infected with an HTLV; individuals who have not yet been infected with HTLV, but who are at risk of becoming infected with HTLV; and individuals who have not yet been infected with HTLV, but who may in the future become infected with HTLV.

### Cancer Treatment

In certain embodiments, the subject methods are suitable for treating individuals diagnosed with a cancer associated with expression of LINES, where such cancers include, but are not limited to, breast cancer, ovarian cancer, melanoma, teratoma, seminoma, prostate cancer and testicular cancer (including teratoma, seminoma, and embryonal carcinoma or mixed tumors composed of one or more of these types). The subject methods are suitable for treating individuals who have been diagnosed with breast cancer; individuals who have been diagnosed with ovarian cancer; and individuals who have been diagnosed with testicular cancer. A subject method of treating cancer is also suitable for treating individuals who have been treated for breast cancer, ovarian cancer, melanoma, prostate cancer, or testicular cancer, and who either failed to respond to the treatment, or responded initially, then relapsed.

### Treatment of an Autoimmune Disorder

In certain embodiments, the subject methods are suitable for treating individuals diagnosed with an autoimmune disorder, where such autoimmune disorders include, but are not limited to, multiple sclerosis, rheumatoid arthritis, systemic lupus erythematosus, and Type 1 diabetes. In some embodiments, the methods are suitable for treating individuals who have been treated for an autoimmune disorder, and who either failed to respond to the treatment, or responded initially, then relapsed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); mAbs, monoclonal antibodies; and the like.

### Example 1

### LINE Peptides Stimulate Cytokine Production in Human Peripheral Blood Mononuclear Cells (PBMCs)

### Methods

Patients. HIV-1 positive volunteers were selected for this study. The study was approved by the local institutional review board and subjects were given written informed consent. Studies were performed on cryopreserved peripheral blood mononuclear cells (PBMCs) from various patient timepoints.

Peptide selection. Candidate LINE epitopes were selected in two ways: (1) based on similarity to peptide sequences found in HIV-1 and (2) based on in silica predicted immunogenicity of the proteins encoded by LINE open reading frame (ORF) ORF 1 and LINE ORF 2.

LINE-1 peptides similar to HIV-1 peptides were identified with BLAST searches (Altschul et al, Nucleic Acids Res. 1997 25(17):3389-402) of LINE amino acid sequences against HIV proteins using the short nearly exact match (e-value=200000, PAM30 matrix, SEG filter OFF, word size=2) parameters for the algorithm. These parameters are appropriate for searching for short regions of highly similar sequence. These parameters reduce the word size of the search from 3 to 2, eliminate the SEG filter, raise the expect value from 10 to 20,000 or above, and include the option of using a less strict similarity matrix (PAM30 as opposed to BLOSUM62). LINE sequence data was obtained from the NCBI databases and the L1base database (Penzkofer et al, Nucleic Acids Res. 2004 33: D498-D500) and compiled into a BLAST database formatted file for searching. HIV HXB-2 reference strain sequence was also compiled into a separate BLAST database formatted file for searching. The short, nearly exact match parameters for the BLAST algorithm facilitate the detection of short matches between peptide sequences, normally overwhelmed by the divergence of the proteins. Overall sequence conservation between proteins is relatively unimportant as a cause of any interaction between the CD8⁺ T-cell responses to endogenous viruses and HIV; rather, such interaction would depend on a high level of similarity or identity of amino acid sequences within epitope-sized regions. Candidate LINE peptides with similarity to known HIV epitopes were selected for peptide synthesis and further testing. Additionally, LINE regions with similarity to HIV proteins outside of known epitopes were also selected for further study.

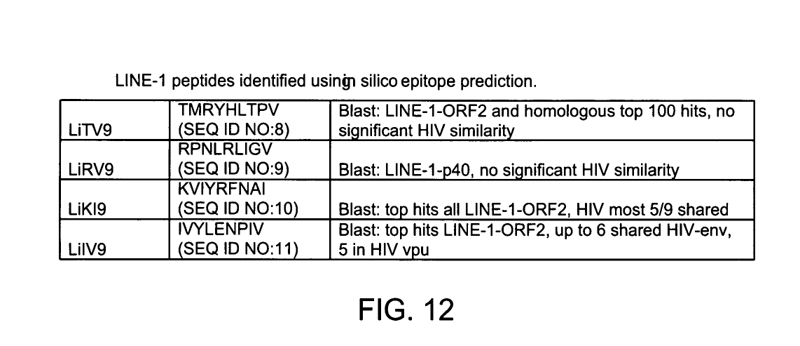

Candidate epitope peptides from LINE-1 were also identified based on in silico predicted immunogenicity of the proteins encoded by LINE-1 ORF 1 and LINE-1 ORF 2. The LINE-1 ORF 1 and ORF 2 sequences were analyzed using the epitope prediction software (NETCTL™) which identifies the sites of proteasomal cleavage, the subset of resulting breakdown products with the best potential to bind the transporter associated with antigen processing (TAP) machinery, and the peptides within those breakdown products with the best binding affinity for different human leukocyte antigen (HLA) molecules (Larsen et al., European Journal of Immunology. 35(8): 2295-303. 2005). The LINE-1 peptides identified according to this method are provided in the table depicted in FIG. 12.

ELISPOT assay. Enzyme-Linked Immunospot (ELISPOT) assay analysis was performed as described in Meiklejohn et al, J. Immunol. Methods (2004) 288, 135-47. Plates were incubated 16 hours at 37° C. Equivalent antigen concentrations were used for HIV and LINE peptide response comparisons. Assays were performed with duplicate wells for each condition, except where cell recovery from archived samples dictated the use of single wells. Plates were counted with an AID ELISPOT reader (Cell Technology,). Spot totals for duplicate wells were averaged, and all spot numbers were normalized to numbers of IFN-γ spot-forming units (SFU) per 1×10⁶ PBMC. Spot values from media control wells were subtracted to determine responses to each peptide. Any resulting peptide values<0 following media subtraction were set to 0 for further analysis.

### Results

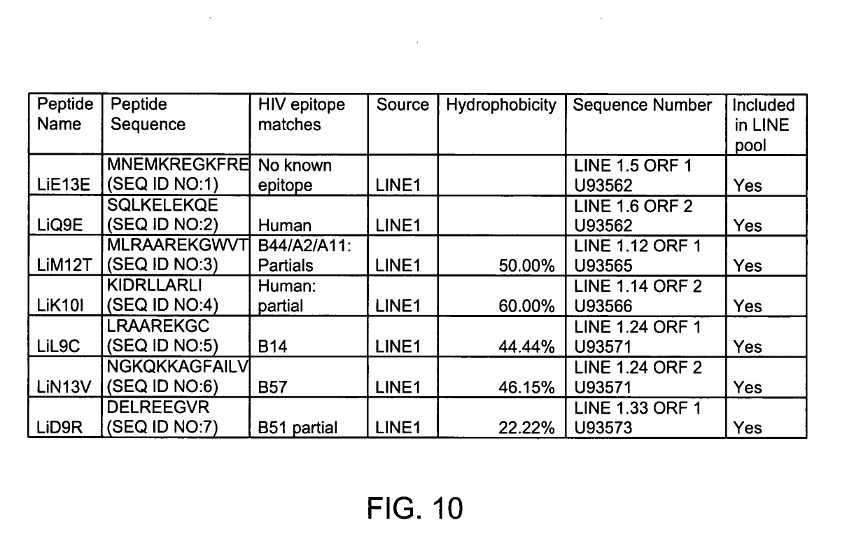

The table presented in FIG. 10 provides candidate LINE polypeptides identified using BLAST searches (Altschul et al, Nucleic Acids Res. 1997 25(17):3389-402) of LINE amino acid sequences against HIV-1 proteins.

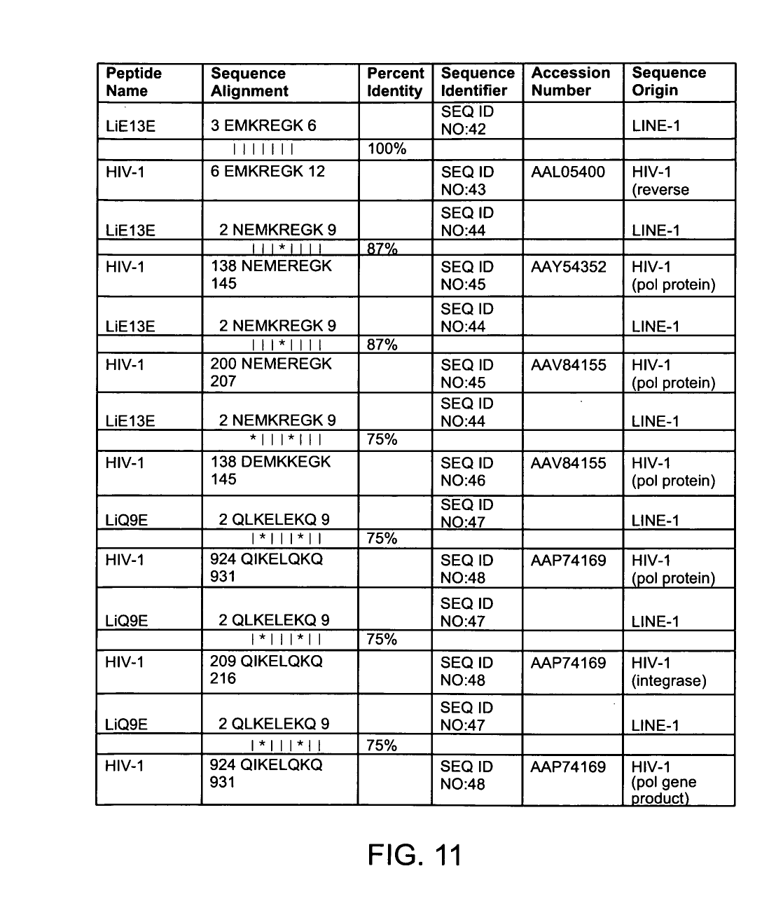

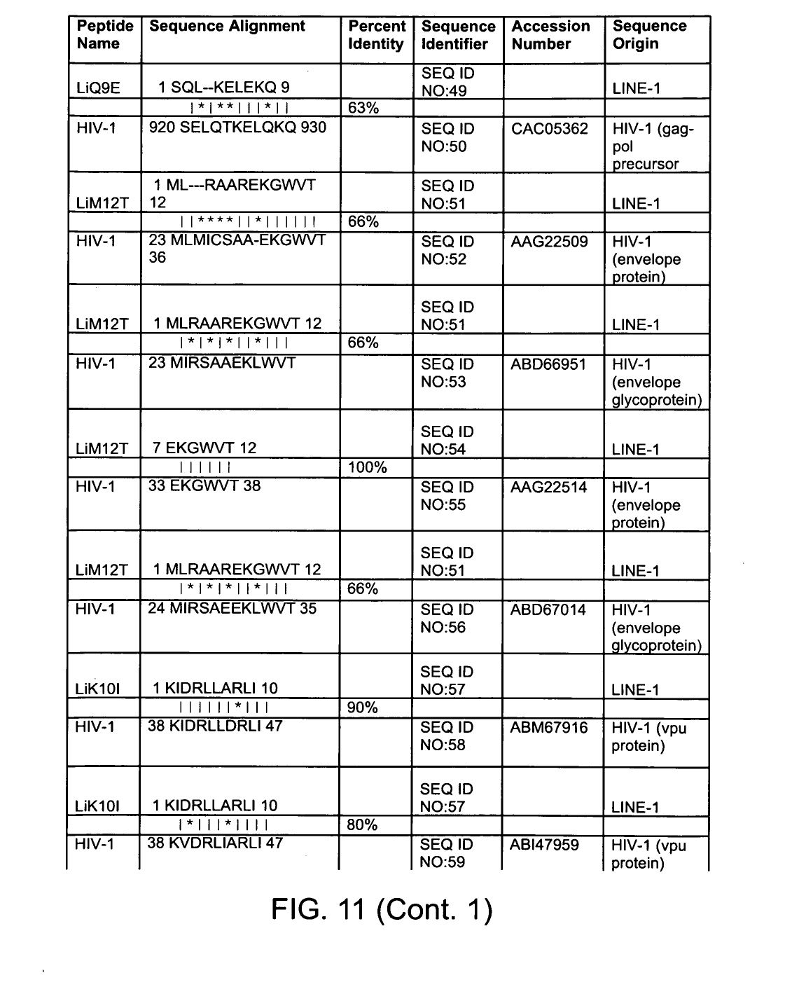

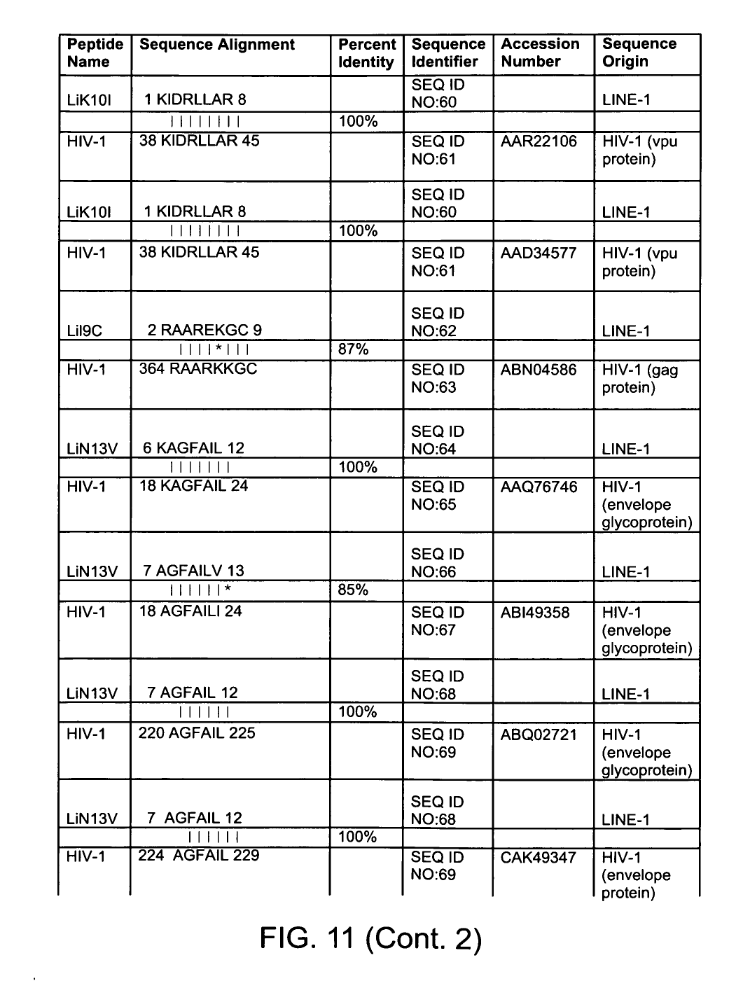

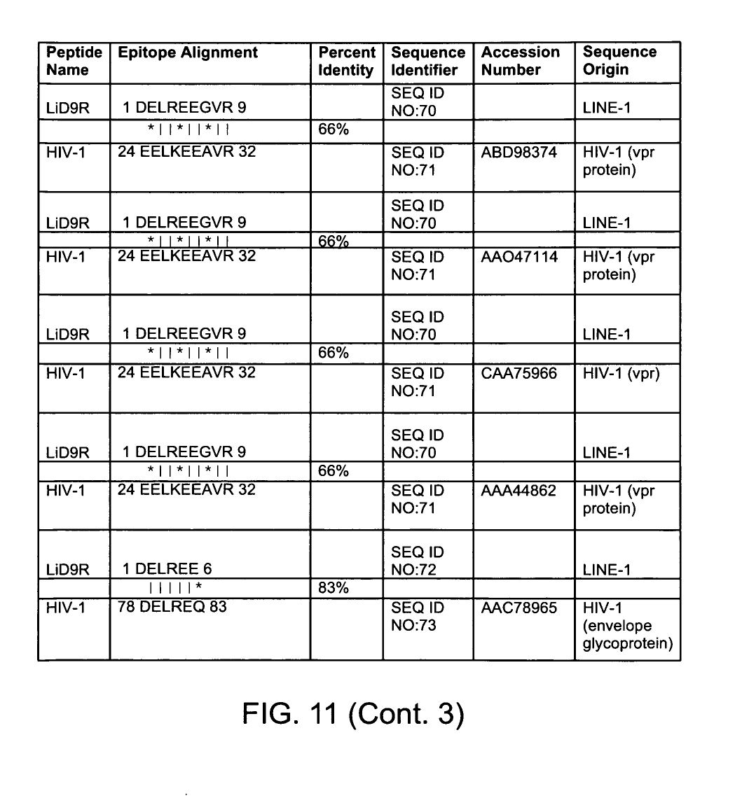

The table presented in FIG. 11 provides exemplary sequence alignments between subject LINE polypeptides and HIV protein sequences resulting from BLAST search of LINE amino acid sequences against HIV-1 proteins. A vertical line indicates an amino acid sequence match and a “*” indicates non-matching amino acids. NCBI database Accession Numbers for the “Subject” HIV-1 sequences are provided. The table presented in FIG. 12 presents LINE-1 peptides identified using in silico epitope prediction.

### Increased Levels of LINE-1 Transcripts in HIV Infected Cells

The ability of HIV-1 infection to influence levels of LINE-1 transcripts in vitro was monitored by comparing levels of transcript expression in HIV-1-infected and mock-infected controls. In parallel, cellular DNA was surveyed by quantitative RT-PCR (qRT-PCR) for increase in LINE-1 genomic copy number, Primary CD4+ T-cells were activated with anti-CD3/anti-CD28 and infected with the R5-tropic strain of HIV-1-81A. By 144 hours post-infection, significant increases in the levels of LINE-1 transcripts were observed relative to a mock-infection control. Increased β-Actin standardized DNA quantification of LINE-1 in 144 hour HIV-1-81A infected cultures was observed. All RNA samples were isolated with Trizol, and treated with DNAse prior to reverse transcription. Trypan blue dye exclusion was similar between HIV-1-81A and mock infected controls.

Detection of LINE-1 Polypeptide Specific Immune Responses in PBMCs Isolated from HIV-1 Infected Subjects

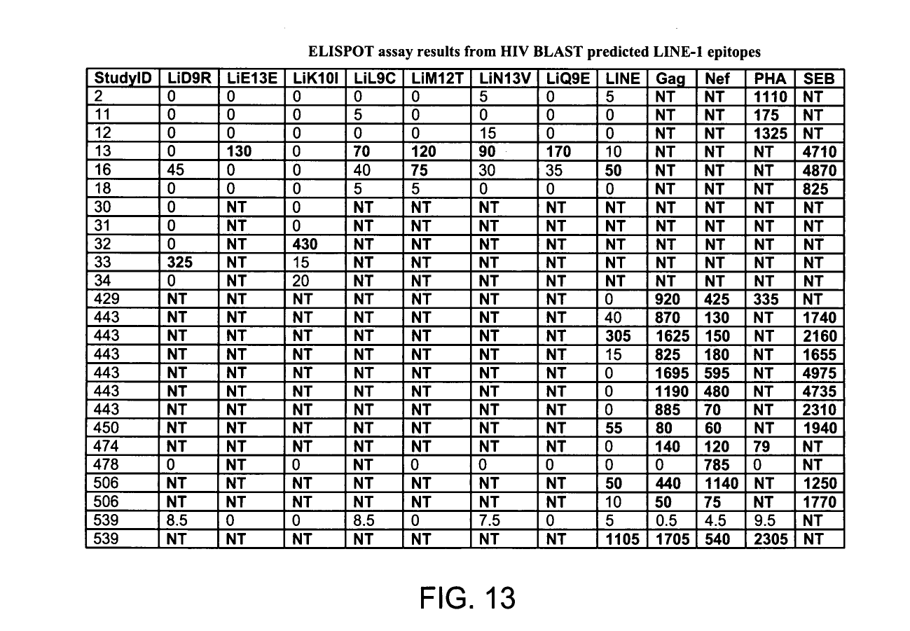

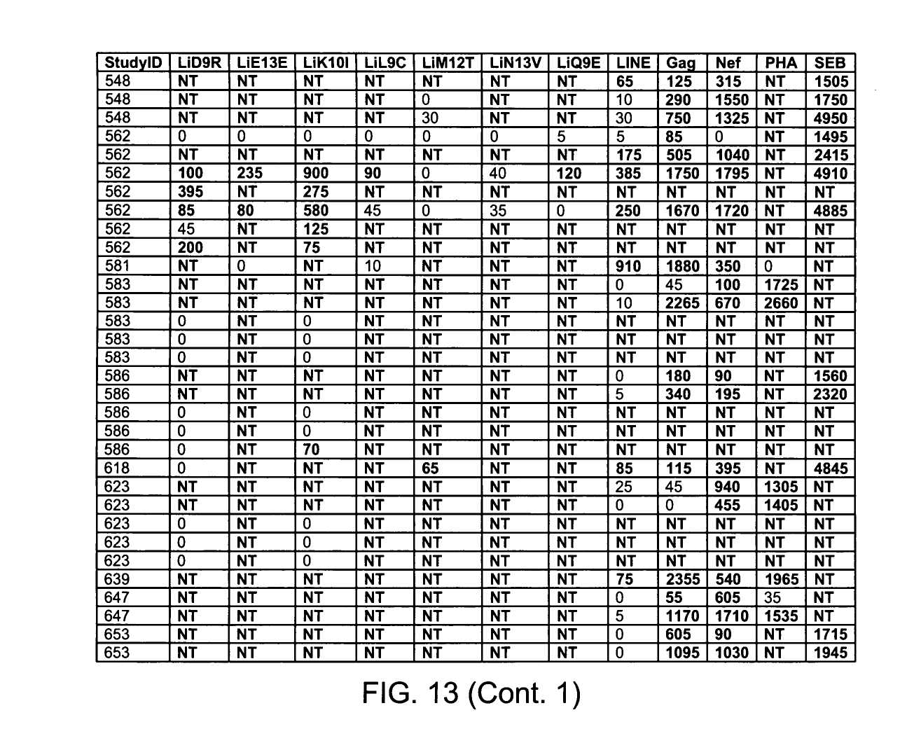

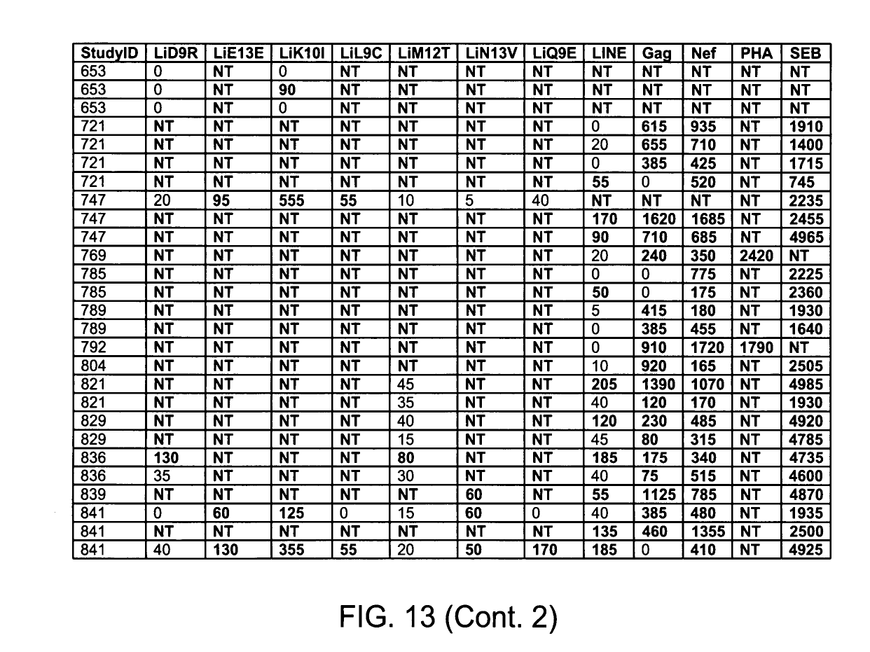

The table presented in FIG. 13 provides ELISPOT assay results for isolated LINE polypeptides LiD9R, LiE13E, LiK10I, Lil9C, Lim12T, LiN13V and LiQ9E; which correspond to SEQ ID NOs: 1-7 respectively. Study IDs 2-34 represent uninfected controls and study IDs 429-841 represent HIV infected individuals. The “LINE” column represents responses to a pool of LINE polypeptides made up of LiE13E, LiK10I, Lil9C, Lim12T, LiN13V and LiQ9E. The Gag and Nef columns represent responses to the Gag and Nef HIV proteins (pools of peptides derived from each protein). The PHA and SEB columns represent responses to positive controls. Results are in spot forming units (SFU) per 1×10⁶ PBMC. Values below 50 are considered to be background and values of 50 and above are considered to be positive responses. An “NT” designation means that a particular patient sample was not tested under the indicated condition on a particular test date. The ELISPOT assay results indicate that cytokine production in response to LINE polypeptide antigenic stimulation can be detected in PBMCs derived from HIV-infected subjects.

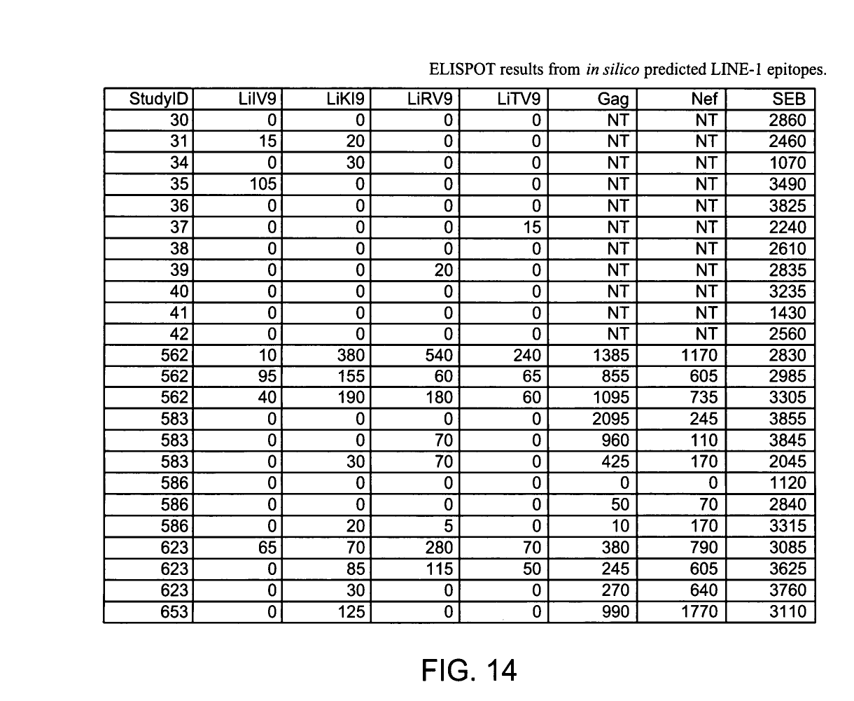

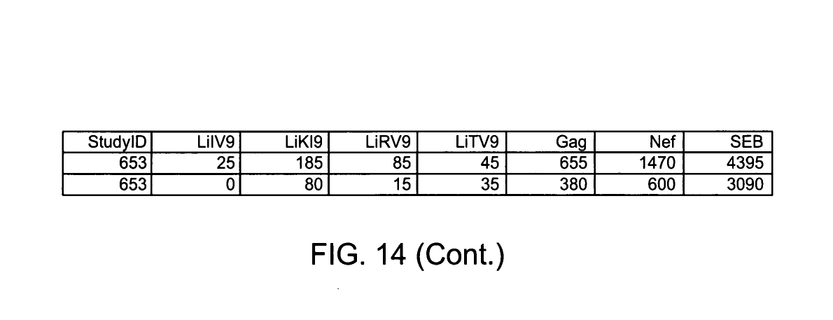

The table presented in FIG. 14 provides ELISPOT assay results for isolated LINE polypeptides LiIV9, LiKI9, LiRV9, LiTV9; which correspond to SEQ ID NOs: 8-11 respectively. Study IDs 30-42 represent uninfected controls and study IDs 562-653 represent HIV infected individuals. The Gag and Nef columns represent responses to the HIV proteins Gag and Nef (pools of peptides derived from each protein). The SEB column represents responses to positive controls. Results are in spot forming units (SFU) per 1×10⁶ PBMC. Values below 50 are considered to be background and values of 50 and above are considered to be positive responses. An “NT” designation means that a particular patient sample was not tested under the indicated condition on a particular test date. The ELISPOT assay results indicate that cytokine production in response to LINE polypeptide antigenic stimulation can be detected in PBMCs derived from HIV-infected subjects.

The data demonstrate an elevation in LINE transcript expression and T cell responses directed at LINE peptides associated with HIV-1 infection. A naturally-arising T cell response against LINEs in HIV-1-infected individuals indicates the feasibility of inducing responses earlier in infection, or in at risk uninfected individuals, as a novel HIV-1 vaccine paradigm. One of the greatest difficulties in HIV-1 vaccine development is overcoming the mutability of the virus, which enables it to evade specific immune responses elicited with a vaccine. LINEs are genome-encoded elements; translation products produced from de-regulated transcription of LINE insertions is expected to be far less variable than HIV-1 proteins. Where LINE antigen production and presentation is a consequence of HIV-1 infection of a cell, the LINE products will serve as a stably recognizable surrogate marker signaling HIV-1 infection to the immune system. Educating the immune system to recognize the LINE surrogate marker through vaccination may induce killing of HIV-1-infected cells, circumventing the need to recognize highly variable HIV-1 antigens.

### Example 2

### L1-Specific CD8⁺ T Cell Recognition of HIV-Infected Cells

### Methods

Subjects. Subjects were selected from participants in the Canadian Immunodeficiency Research Collaborative (CIRC) Cohort, Toronto, Canada, and the SCOPE Cohort, University of California San Francisco (UCSF). Chronic progressors were defined as individuals infected with HIV-1 for >1 year with CD4⁺ T cell count decline>50 cells/mm³/year. Viral controllers were defined as individuals infected with HIV-1 >1 year, no evidence of CD4⁺ T cell count decline, and viral load<5,000 copies/ml bDNA. This study was approved by the University of Toronto Institutional Review Board and by the UCSF Committee on Human Research and subjects gave written informed consent. Studies were performed on cryopreserved PBMCs immediately after thawing.

Immunoprecipitation/Western Blot Analysis of L1-p150 Protein Expression. PBMC were obtained from an HIV-1-uninfected donor. CD4⁺ T cells were isolated using Easysep (Stemcell Technologies), and stimulated for 48 hours with monoclonal antibodies (mAbs) to CD3 and CD28 (ebiosciences) in RPMI media supplemented with 10% FBS, glutamine, penicillin/streptomycin, and 50 U/ml IL-2 (Hofmann-La Roche). These cells were divided into two equal aliquots, one of which was infected with 0.05 MOI of HIV-1-NL4-3 and the second of which was maintained as a mock-infected control. At the indicated time-points (48 and 170 hours) post-infection 10⁶ cells were lysed with radioimmunoprecipitation assay (RIPA) buffer supplemented with complete protease inhibitor cocktail (Roche). Immunoprecipitation was performed using the Seize Protein G Immunoprecipitation Kit (Pierce Biotechnology), following manufacturer's instructions. Proteins were immunoprecipitated from 5 mg whole cell lysate proteins using 40 μg of the goat polyclonal anti-L1-p150 S19 (Santa Cruz Biotechnologies). These eluates were each concentrated to 50 μl using a YM-50 50 kDa molecular weight cut-off microcon (Millipore), divided into 3 equal portions, and separated by reducing sodium dodecyl sulfate-polyacrylamide gel electrophoresis (SDS-PAGE) on 3 separate gels using the NuPage system (Invitrogen) following manufacturer's protocol. Proteins were transferred to poly(vinylidene fluoride) (PVDF) membranes for 1 hour at 100V. Western blots were performed by standard procedures probing with anti-L1-p150 S19 and C16 antibodies (Santa Cruz Biotechnologies) at 1:200 dilutions followed by donkey anti-goat IgG-horse radish peroxidase (HRP) (Jackson Immunoresearch) at a 1:10,000 dilution, and with anti-HIV-1-p24 anti-serum (NIH AIDS Reagent Program cat #4250) at a 1:10,000 dilution follow by goat anti-rabbit IgG-HRP (Jackson Immunoresearch) at a 1:10,000 dilution.

Epitope Selection and Peptide Synthesis. The consensus nucleotide sequence of ‘hot’ L1 elements presented by Brouha et al. ((2003)Proc. Natl. Acad. Sci. USA 100:5280) was translated to derive L1 ORF1 and ORF2 amino acid sequences. These sequences were uploaded into the NetCTL algorithm (on the internet at cbs.dtu.dk/services/NetCTL/) to predict both A2 and B7 superfamily epitopes. The top scoring predicted epitopes for each of these superfamilies were cross-referenced with the goal of selecting peptides with high scores for both, and thus the potential for broad reactivity. Peptides were synthesized by standard 9H-fluoren-9-yl-methoxycarbonyl (FMoc) chemistry. See, e.g., “Fmoc Solid Phase Peptide Synthesis: A Practical Approach” W. C. Chan and P. D. White, eds. (2000) Oxford Univ. Press.

ELISPOT. ELISPOT assays were performed using standard procedures (reference). Cells were plated at 10⁵ PBMC/well. Individual peptides representing L1 epitopes were used at 10 μg/ml, the CMV pp65 pool at 5 μg/ml/peptide, and the HIV-1 big pool at 1 μg/ml/peptide. Incubations were performed for 16 hours at 37° C., 5% CO₂.

CD8⁺ T Cell Cloning. Two different cloning methods were employed in this study: a standard protocol where antigen-specific cells are enriched by in vitro expansion, and a magnetic cell separation (MACS) method where antigen-specific cells were enriched by magnetic capture.

Briefly, CD8⁺ T cells were stimulated with peptide for 16 hours in the context of whole ex vivo PBMC. CD8⁺ T cells that produce cytokine (e.g IFN-γ) in response to this stimulation were labeled with a cytokine capture reagent, allowing for specific labeling with magnetic beads (IFN-γ-secretion assay, Miltenyi Biotec). These antigen-specific CD8⁺ T cells were then enriched from the bulk population by magnetic separation, plated over serial dilutions, and expanded using irradiated allogeneic PBMC and B cell lymphoma lines with mAbs to CD3 (clone OKT3) and CD28 (clone CD28.7) (both from ebioscience).

Following enrichment, cells were plated over a serial dilution on irradiated feeder cells. Antigen-specific clones at limiting dilution were subjected to a second round of limiting dilution before being expanded and maintained in RPMI 10% fetal bovine serum (FBS) with 50 U/ml IL-2 (Hoffman La-Roche) and biweekly addition of irradiated feeder cells. The clone “L1-3O” was obtained by the MACS method.

HIV-1 Infections. NL4-3 and 81A stocks of HIV-1 were prepared by transfection of HEK293T cells using FuGene 6 (Roche) and collection of supernatants 4 days post. Primary isolates representing diverse clades of HIV-1 were used as received from the NIH AIDS reagent program. Activated, primary CD4⁺ T cell targets were prepared by standard methods. In all experiments, except the p24 suppression assay and the immunoprecipitation/western blot, we employed magnetofection to obtain high-level infections as has been previously described (Sacha, J. Immunol, 2007; 178: 2746-2754). For the p24 suppression assay 0.02 multiplicity of infection (MOI) of HIV-1 were added to target cells for 1 hour and then washed off, and infection was allowed to proceed at 37° C., 5% CO₂.

Recognition Assays. Testing CD8⁺ T cell clones and lines for recognition of HIV-1-infected cells was performed as previously described (Sacha, J. Immunol, 2007; 178: 2746-2754). Briefly, approximately 2 weeks after restimulation, CD8⁺ T cell effector lines and clones were washed with phosphate buffered saline (PBS) and combined with HIV-1 and mock infected CD4⁺ T cell targets at a ratio of 1:1 (at least 2×10⁴ of each). For kinetics assays, infected cells were added at the stated time post-infection. Effectors and targets were cultured together at 37° C., 5% CO₂ for 1 hour. In experiments which indicated CD107a as a readout (ex. TO2) 5 μg/ml of PE-conjugated anti-CD107a mAb (BD) was added at this stage (prior to 1 hour co-culture). Brefeldin A was then added to a final concentration of 10 μg/ml and incubations were allowed to proceed for an additional 5 hours. Cells were then surface stained with mAbs to CD4 and CD8, permeabilized using cytofix/cytoperm (Becton Dickinson; BD), and then stained for either interferon-gamma (IFN-γ) or tumor necrosis factor-alpha (TNF-α) using mAbs (BD). Flow cytometry was performed on either a LSRII or FACSCalibur instrument (both BD).

Elimination Assays. Target autologous, or HLA-mismatched CD4⁺ T cells were synchronously infected with HIV-1 by magnetofection (see above). Two hours post-infection, target cells were mixed 1:1 with washed CD8⁺ T cell effector lines and clones (at least 3 weeks after most recent restimulation). Targets and effectors were co-cultured for 48 hours at 37° C., 5% CO₂. Cells were surface stained with fluorochrome-conjugated mAbs to CD4 and CD8 (BD), and an amine red viability dye (Invitrogen). Following permeabilization with cytofix/cytoperm (BD), cells were stained with a fluorescein isothiocyanate (FITC)-conjugated monoclonal antibody to HIV-1-Gag (clone Kc57, Beckman Coulter). Analysis was performed on an LSRII flow cytometry instrument (BD).

Suppression of p24 Production. Autologous and human leukocyte antigen (HLA) mismatched CD4⁺ T cell targets were infected with HIV-1 NL4-3 (see HIV-1 infections, above), and plated at 2×10⁴ cells/well in 96 well round-bottom plates. CD8⁺ T cell effectors were taken 3 weeks following latest restimulation, washed 2× with PBS and added to wells at desired ratios. Effector/target mixtures were incubated in a total volume of 200 μl of RPMI—10% FBS supplemented with 50 U/ml IL-2. At 9 days post infection 100 μl of media was removed and assayed by p24 enzyme linked immunosorbent assay (ELISA) (NCI Frederick) following manufacturer's instructions. Concentration of p24 was calculated based on a standard curve using p24 standards supplied by NCI Frederick.

HLA-A,B,C Blockade. Anti-HLA-A,B,C mouse monoclonal IgG1 antibody (clone G46-2.6), and mouse IgG1 control antibody were obtained from BD Biosciences. Target and effector populations were prepared as described above. Prior to co-culturing targets and effectors, infected target cells were incubated with 10 μg/ml of anti-HLA-A,B,C or isotype control for 30 minutes at 37° C., 5% CO₂. Effector and target cells were then co-cultured and assessed for recognition as described above.

### Results

L1 p150 is Detectable in HIV-1 Infected, but not Uninfected Primary CD4⁺ T Cells.

CD4⁺ T cells were enriched from PBMC from HIV-1-uninfected individuals and stimulated for 48 hours with monoclonal antibodies (mAbs) to CD3 and CD28. These cells were subsequently infected with HIV-1-NL4-3 using a standard infection protocol with 0.05 MOI or maintained as a mock infection control (see methods). Cells were lysed at 48 and 170 hour post-infection time-points with RIPA buffer supplemented with complete protease inhibitor cocktail (Roche). Proteins from whole cell lysates were separated by SDS-PAGE and analyzed by western blot using the S19 and C16 anti-L1-ORF2 polyclonal antibodies (pAbs) from Santa Cruz Biotechnology. These studies failed to detect any bands at the expected molecular weight of 150 kDa. To increase the sensitivity of our assay we proceeded to include an immunoprecipitation step prior to western blot analysis. 5 mg of protein from whole cell lysates of the above samples were immunoprecipitated using the seize classic (G) immunoprecipitation kit (Pierce) with anti-L1-p150 S19 antibody (Santa Cruz Biotechnology). The retained fraction was separated by SDS-PAGE and then probed in western blots using either the S19 antibody, or the anti-L1-p150 C16 antibody (Santa Cruz Biotechnology).

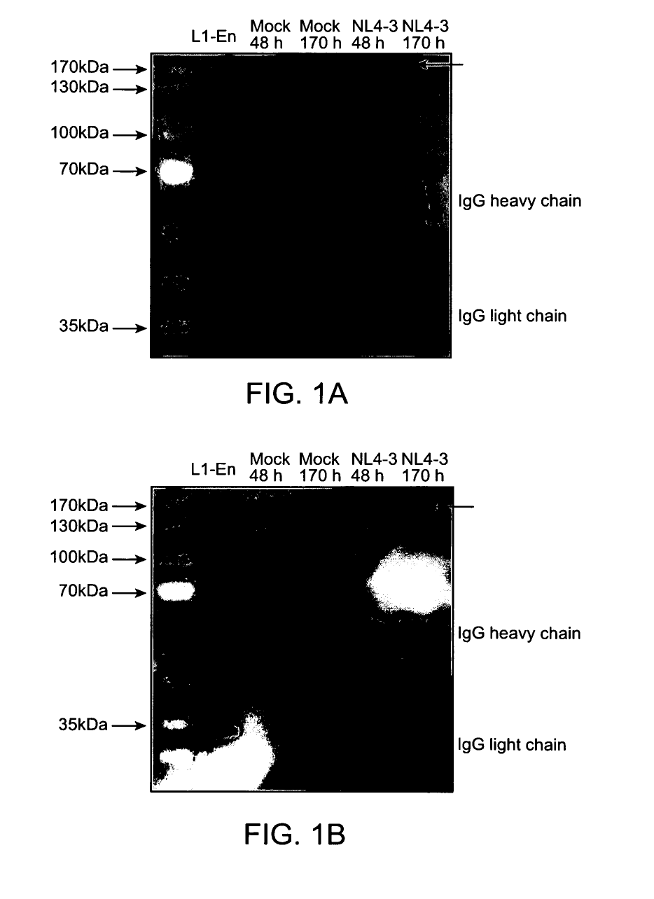

A band was observed at the expected L1-p150 molecular weight of 150 kDa in the 170 hour post-HIV-1-NL4-3 infection sample when the western blot was probed with either S19 or C16 (FIGS. 1A,B). This band was absent from both of the mock-infection samples, and from the 48 hour post HIV-1-infection sample. No band was detected when the L1-p150-S19 immunoprecipitated fraction was probed with anti-HIV-1-Gag. These data demonstrate that in vitro HIV-1-infection of primary CD4⁺ T cells results in expression of L1-p150. The fact that only a faint band was detected after immunoprecipitation and western blot indicates that L1-p150 is expressed only at low levels in HIV-1-infected cells. This is consistent with recent data showing that the expression of high levels of L1-p150 is toxic (Wallace, Gene. 2008; 418(1-2): 75-81). The expression of low levels of L1-p150 may, however, be sufficient to stimulate cellular immune responses.

FIGS. 1A and 1B. L1-p150 is expressed in HIV-1-Infected Primary CD4⁺ T cells. Primary CD4⁺ T cells from HIV-1-uninfected individuals were activated by stimulation with anti-CD3/CD28 for 48 hours. Cells were then divided into two aliquots and either infected with 0.05 MOI of HIV-1-NL4-3, or maintained as a mock infected control. Aliquots were taken at 48 and 170 hours post infection (or mock infection) and lysed in RIPA buffer with protease inhibitors. Lysates were immunoprecipitated with the polyclonal anti-L1-p150 antibody S19 (Santa Cruz Biotechnologies). The eluates from these immunoprecipitations were concentrated, separated by SDS-PAGE, and probed in western blot either with the anti-L1-p150 antibody S19, or with the anti-L1-p150 antibody C16 (Santa Cruz Biotechnologies). FIG. 1A. Western blot for samples immunoprecipitated with the S19 antibody and probed with the C16 antibody. FIG. 1B. Western blot for samples both immunoprecipitated and probed with the S19 antibody. Lane 1 for each blot is loaded with the PageRuler protein ladder (Fermentas), lane 2 is loaded with the endonuclease domain of L1-p150 expressed in e. coli (positive control for p150 detection, but runs at a much smaller molecular weight as it is only a fragment of L1-ORF2p). The dark bands at 55 kDa and 25 kDa represent the heavy and light chains of the antibody used in the immunoprecipitation. Since both S19 and C16 are derived from goats the secondary antibody reacts with both the antibody used in the probing and in the immunoprecipitation. Since the positive control L1-endonuclease domain was not immunoprecipitated, these bands are absent from this lane.

Cellular Immune Responses to L1 are Detectable in PBMC from HIV-1-Infected Individuals.

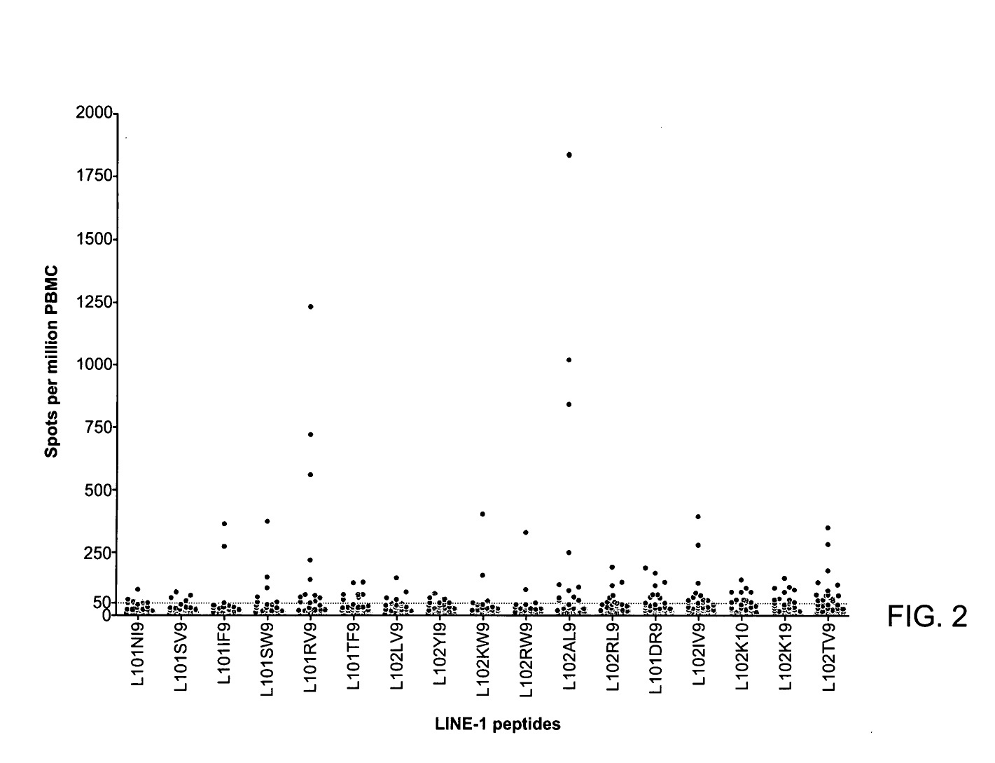

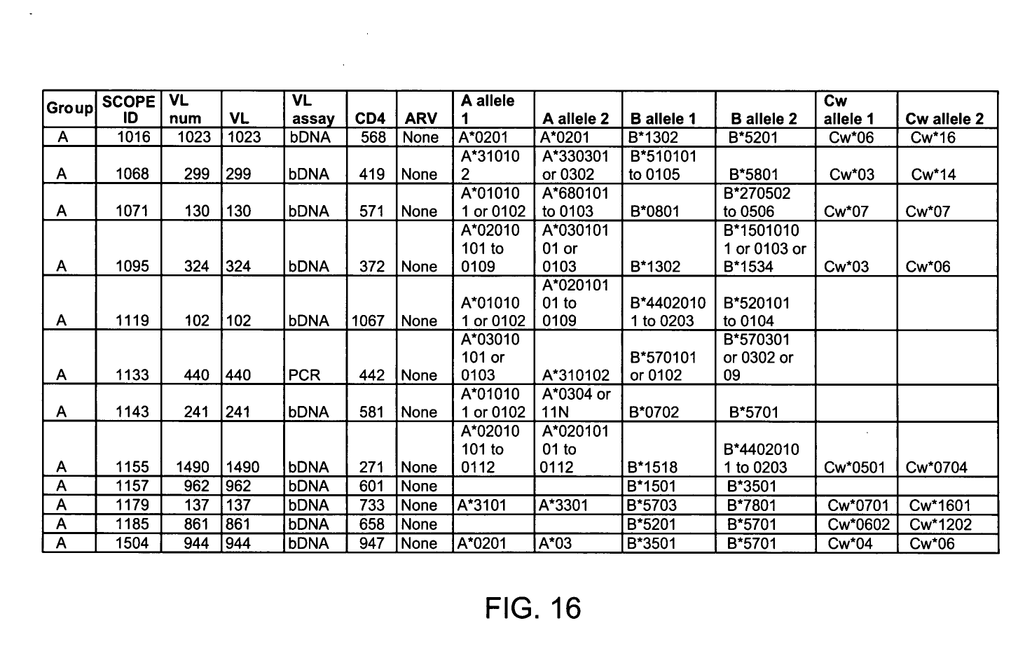

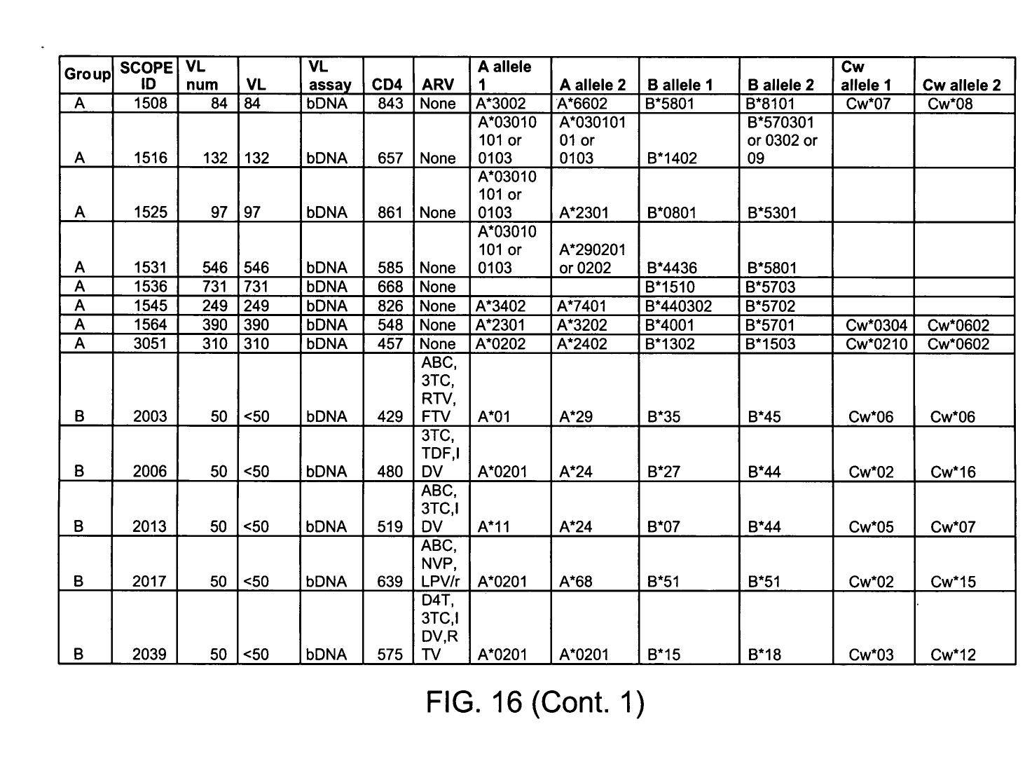

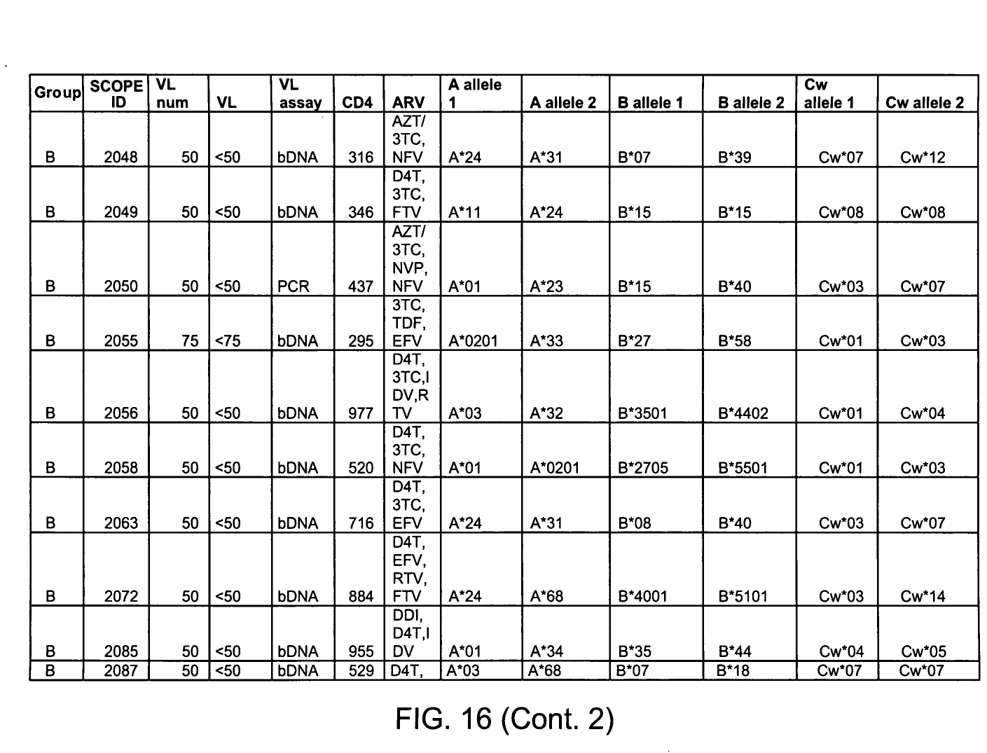

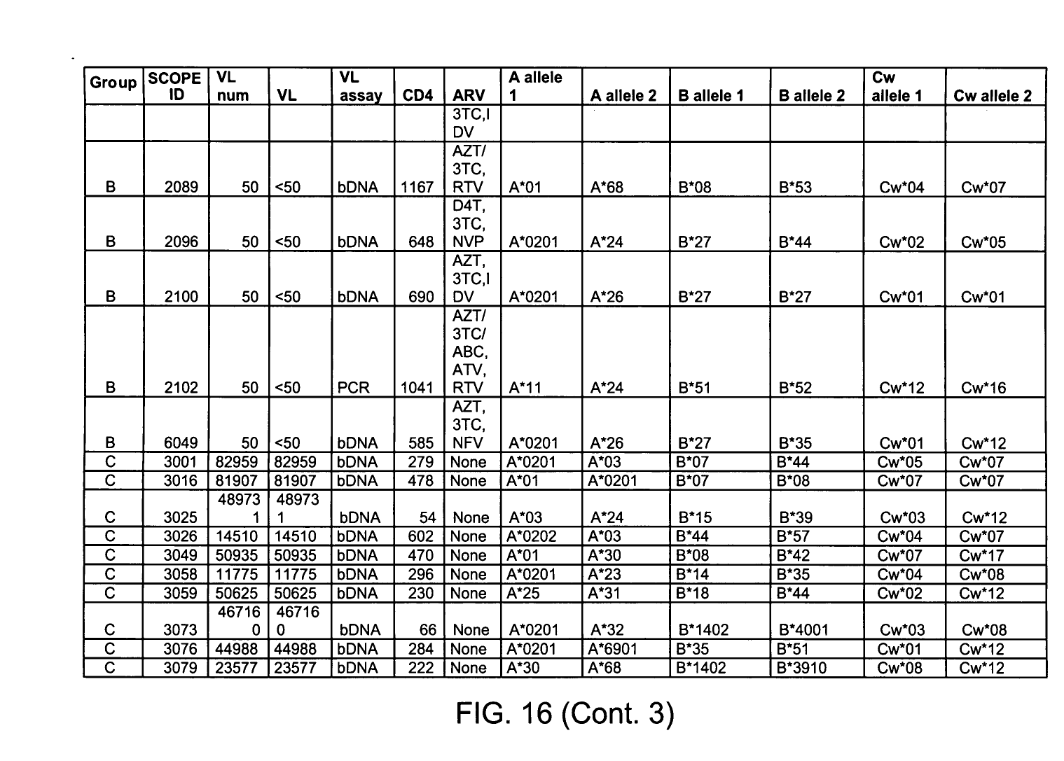

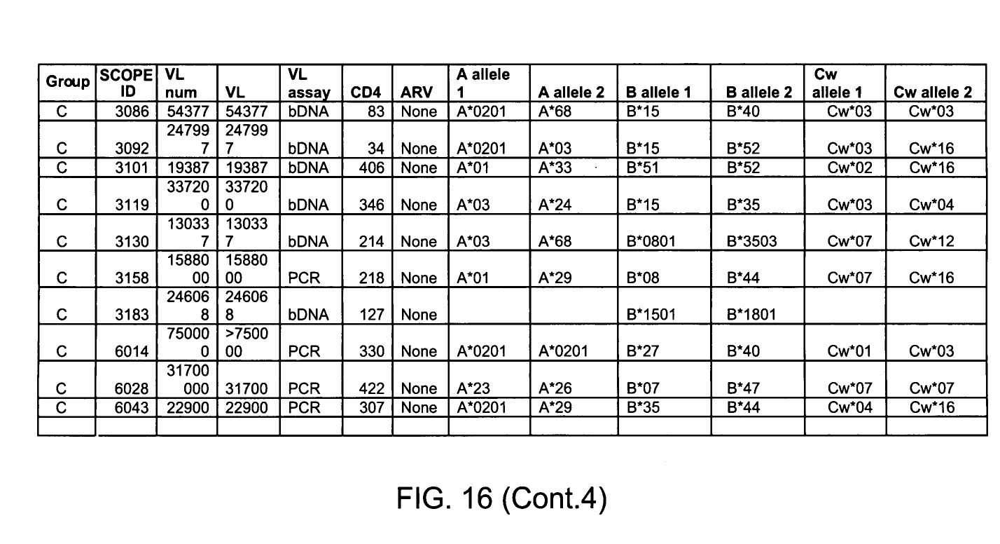

Consensus “hot” L1 p40 and p150 sequences were analyzed using the NetCTL algorithm to predict potential HLA A02, B07, and B58 restricted T cell epitopes. Peptides corresponding to 7 p40 and 10 p150 epitopes were manufactured, and tested for their ability to stimulate IFN-γ production in PBMC from 60 HIV-1-infected individuals with diverse clinical characteristics (SCOPE cohort, see the table presented in FIG. 13 for peptide sequences, and the table presented in FIG. 16 for subject characteristics), as well as 27 low-risk HIV-1-uninfected individuals by ELISPOT. Frequent IFN-γ responses to both p40 and p150 derived epitopes in HIV-1-infected individuals, and a lack of responses in HIV-1-uninfected individuals, were observed (FIG. 2).

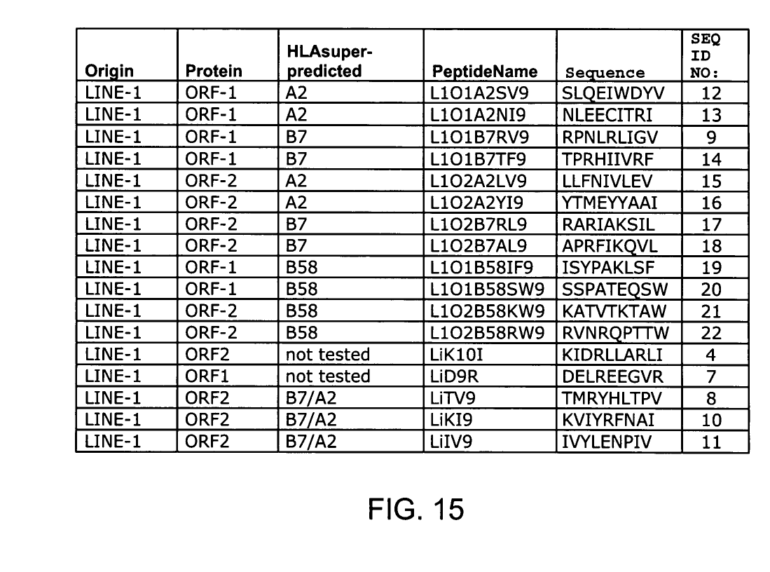

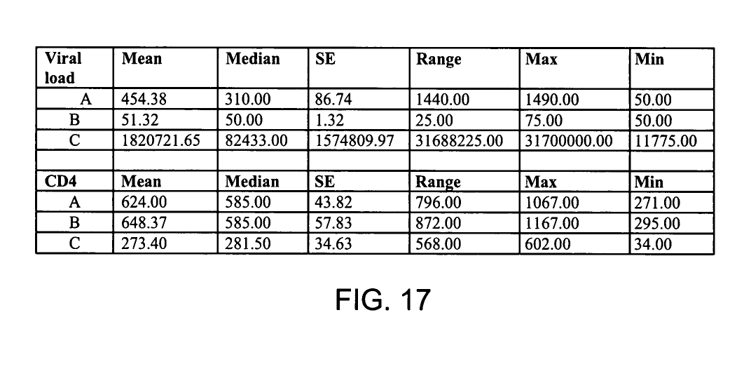

FIG. 2. Immune Responses to L1 in Peripheral Blood of HIV-1-Infected Individuals but not Uninfected Individuals. Cryopreserved PBMC from 60 HIV-1-infected individuals and 27 HIV-1-uninfected subjects were tested at 10⁵ cells/well in an IFN-γ ELISPOT assay for responsiveness to synthetic L1 p40 (ORF1p) and p150 (ORF2p) peptides. ORF1p peptides are indicated by the “L1O1” prefix while ORF2p are indicated by “L1O2”. Peptide sequences and characteristics are given in the table presented in FIG. 15. HIV-1-infected subject information is given in the tables presented in FIGS. 16 and 17.

L1 p150-Specific CD8⁺ T Cell Clones Specifically Recognize HIV-1-Infected Cells in an HLA-Restricted Manner.

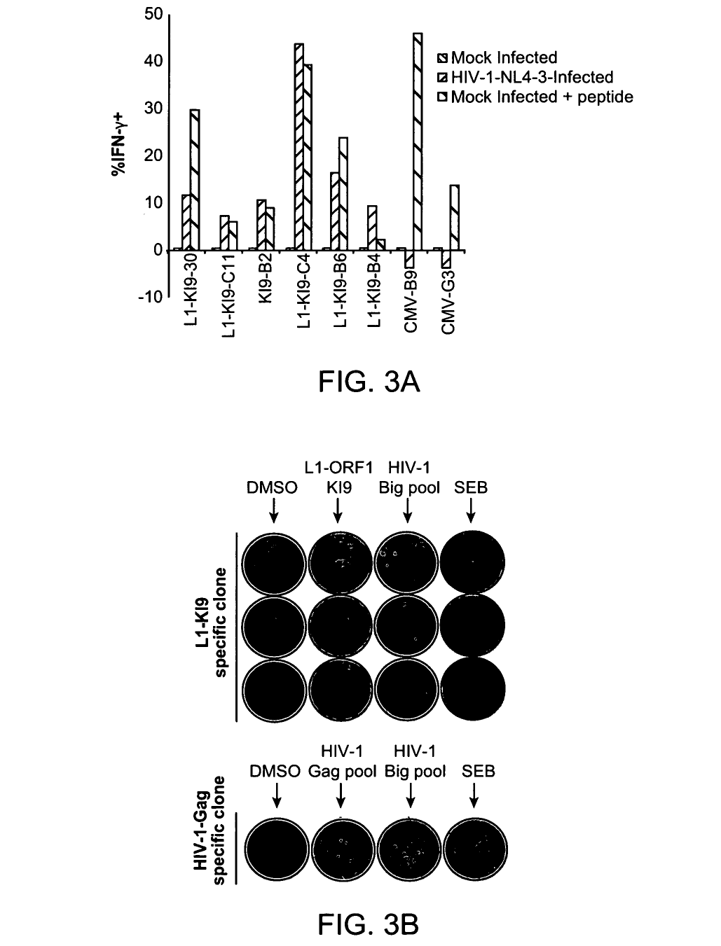

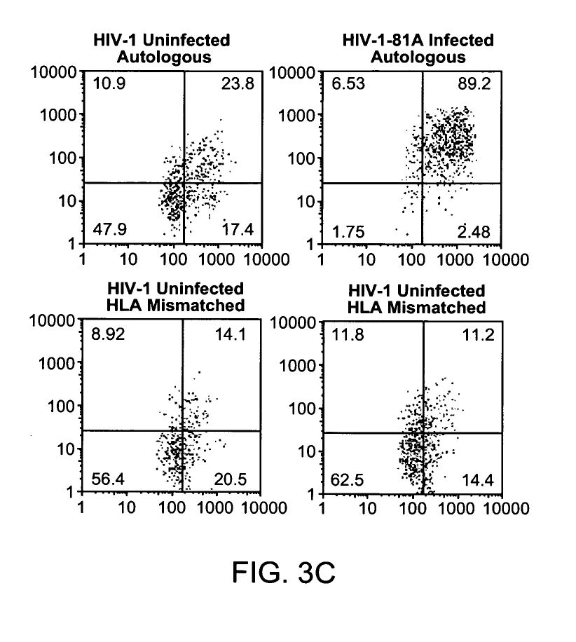

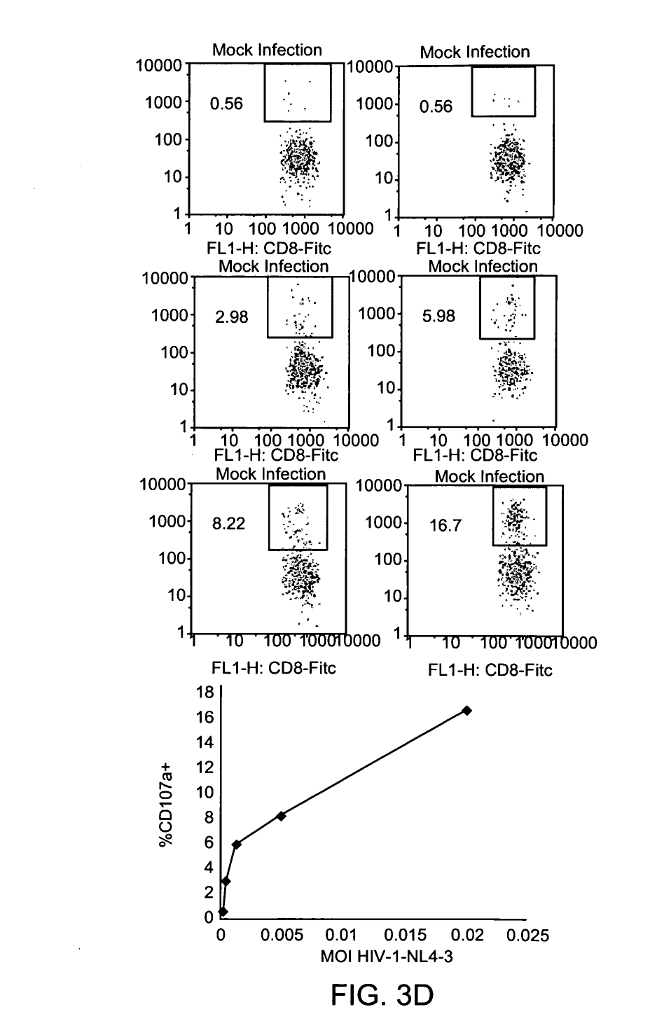

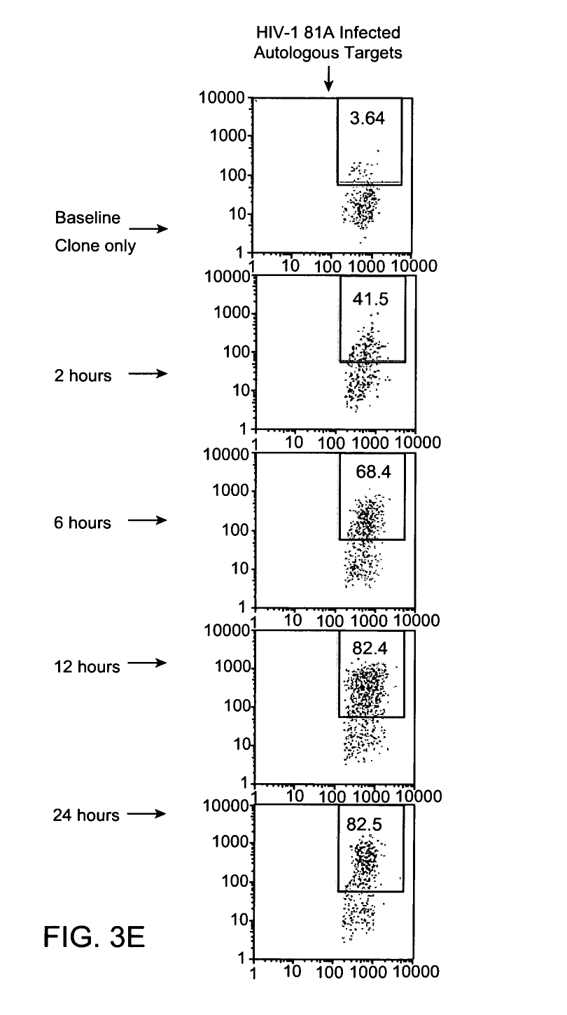

Six CD8⁺ T cell clones specific for the L1-p150 epitope ‘KVIYRFNAI’ (KI9; SEQ ID NO:10), and two CMV-pp65-specific CD8⁺ T cell clones were obtained from an HIV-1-infected individual who had maintained an undetectable viral load for >10 years without HAART. These clones were tested for their ability to specifically respond to autologous HIV-1-infected cells using an intracellular cytokine staining based recognition assay that has been previously described (Sacha, J. Immunol, 2007; 178: 2746-2754). Briefly, CD4⁺ T cells were enriched from autologous PBMC and either infected with HIV-1-NL4-3, or maintained as a mock infected control. Clones were co-cultured with mock infected, HIV-1-NL4-3 infected targets, and mock infected targets pulsed with cognate peptide for 6 hours, and levels of CD107a staining and IFN-γ production from clones these were assessed by flow cytometry. These data are summarized in FIG. 3A.

Recognition of peptide-pulsed mock-infected targets by all L1-p150-KI9 and CMV-pp65-specific clones tested was observed. For each of the six L1-p150-KI9-specific CD8⁺ T cell clones we observed robust recognition of HIV-1-infected target cells. This was contrasted by a lack of recognition of HIV-1-infected target cells by CMV-pp65-specific CD8⁺ T cell clones. FIG. 3B-E displays detailed data for the L1-p150-KI9-specific CD8⁺ T cell clone “L1-3O”. The KI9 epitope does not bear a high degree of identity to any sequence within HIV-1-NL4-3 (at best 1/9 amino acid identity by clustalw alignment with NL4-3 sequence: NCBI accession M19921).

It was not anticipated that the L1-p150-KI9-specific CD8⁺ T cell clones should directly cross-recognize HIV-1-derived peptides. However, this possibility was directly examined by testing whether the L1-3O-KI9 clone would respond to a pool of overlapping 15mers spanning all HIV-1 consensus sequence gene products, “HIV-1-Big Pool”, by IFN-γ ELISPOT. This clone failed to recognize the HIV-1-Big Pool, while exhibiting strong recognition of its cognate L1-p150 peptide. In parallel an HIV-1-Gag-specific clone obtained from the same individual was tested for the ability to recognize both HIV-1-Gag pool and HIV-1-Big Pool and observed similar magnitude responses to both pools (FIG. 3B). Clone L1-30 was next tested for recognition of autologous and HLA-mismatched HIV-1-NL4-3 infected and mock infected target cells. Strong recognition of autologous HIV-1 infected target cells was observed, manifesting as a high-level of CD107a staining, and production of IFN-γ (89.2% CD107a⁺ IFN-γ⁺) (FIG. 3C). This was contrasted by a low frequency of either CD107a⁺ or IFN-γ⁺ clone cells following co-culture with mock infected autologous targets (23.8% CD107a⁺ IFN-γ⁺), or with either mock or HIV-1 infected HLA-mismatched targets (14.1% and 11.2% CD107a⁺ IFN-γ⁺ respectively). This experiment was repeated an additional 4 times with similar results. Taken together these data support that L1-p150-specific T cells recognize HIV-1-infected cells in an HLA-restricted manner without directly recognizing HIV-1-peptides.

To determine whether recognition of autologous cells was dependent upon the dose of HIV-1, clone L1-3O effector cells were mixed with equal numbers of autologous CD4⁺ T cell targets infected with a 4-fold serial dilution of HIV-1-NL4-3 with MOIs ranging from 1.6×10⁻⁴ to 2.0×10⁻², as well as with a mock-infected control. As above, a mAb to CD107a was included in the co-incubation, and CD107a staining was assessed by flow cytometry as a readout of clone responsiveness. CD107a staining increased in a dose dependent manner with increasing titres of HIV-1, ranging from 0.56% CD107a⁺ in the mock infection, to 16.7% CD107a⁺ with an MOI of 2×10⁻² (FIG. 3D). The kinetics by which L1-specific clones recognized HIV-1 infected cells were examined (FIG. 3E). High titres of HIV-1, and a previously described magnetofection protocol, were employed to synchronize HIV-1 infection of target cells (Sacha, J. Immunol, 2007; 178: 2746-2754) (see methods). At various timepoints post-infection L1-specific CD8⁺ T cell clones were mixed with infected targets for 1 hour and then treated with brefeldin A (BFA) and incubated for an additional 5 hours. The times indicated in FIG. 3E represent hours post-infection that targets and clones are first mixed. The addition of BFA prevents the trafficking of newly produced peptide:MHC complexes to the cell surface, hence limiting the epitopes presented to T cells over the culture to those present at the time of BFA addition. As with previous experiments, CD107a and cytokine staining were used as readouts of clone stimulation. The data shown in FIG. 3E presents TNF-α staining on clone cells—using CD107a or IFN-γ as readouts results in comparable kinetics. Recognition of targets by L1-specific CD8⁺ T cell clones were consistently observed within 2 hours of HIV-1 infection, with responses peaking at 82.4% TNF-α⁺ at 12 hours.

FIGS. 3A-E. L1-specific CD8⁺ T cells specifically recognize HIV-1 infected cells within 2 hours of infection. A. Six L1-specific CD8⁺ T cell clones and two CMV-specific CD8⁺ T cell clones were obtained from an elite controller HIV1-infected individual by a limiting dilution methodology. These clones were co-cultured with autologous CD4⁺ T cell targets either: infected with HIV-1-NL4-3, maintained as a mock infection control, or pulsed with cognate peptide. Recognition of target cells was assessed by intracellular cytokine staining flow cytometry with mAbs to CD4, CD8, CD107a, and IFN-γ. Shown are % IFN-γ producing clone cells (CD8⁺ gate) after subtracting background response to mock infected cells. B. Clones were confirmed for responsiveness to cognate peptide, and confirmed for cross-reactivity to HIV-1 big pool (comprised of 15mer peptides spanning all HIV-1 gene products) by IFN-γ ELISPOT. Shown are the results from the L1-ORF-2-KVIYRFNAI (SEQ ID NO:10) epitope-specific clone ‘L1-3O ’, tested in triplicate. In parallel, also shown, an HIV-1-Gag-specific CD8⁺ T cell clone was tested for responsiveness to Gag pooled peptides, and HIV-1 big pool. For both clones Staphylococcus Enterotoxin B (SEB) was used as a positive control. C. Shown is representative recognition assay data for the L1-p150-KVIYRFNAI (SEQ ID NO:10)-specific CD8⁺ T cell clone L1-KI9-3O. D. The L1-specific CD8⁺ T cell clone L1-KI9-3O was tested for recognition of autologous CD4⁺ T cell targets infected with serial dilutions of HIV-1-NL4-3 from 0 to 0.02 MOI. Recognition was assessed by flow cytometry, staining for the degranulation marker CD 107a with a mAb added at the initiation of the stimulation period. E. Clone L1-KI9-3O was tested for recognition of autologous CD4⁺ T cell targets at 0, 2, 6, 12, and 24 hours post-infection with HIV-1-NL4-3. Synchronous HIV-1 infection of targets was achieved by magnetofection (see methods). MHC-I presentation of peptides was halted at the stated time-point by addition of Brefeldin A and stimulation was allowed to proceed for an additional 5 hours.

L1 p150-specific CD8⁺ T Cell Clones Specifically Eliminate HIV-1-Infected Cells and Suppress Viral Replication.

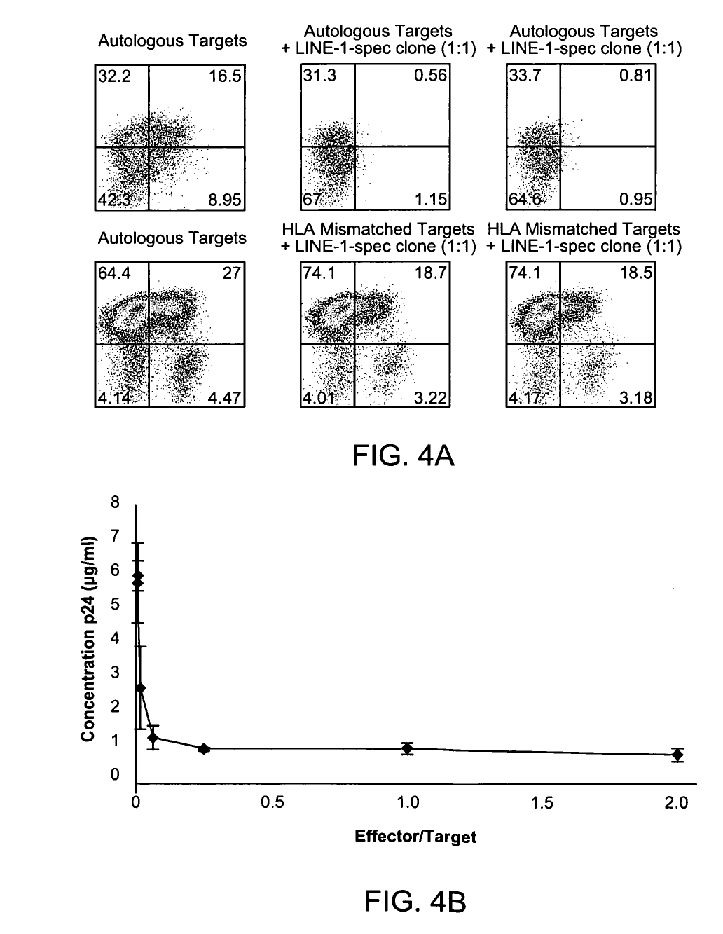

It was determined if the recognition of HIV-1 infected cells by L1-specific CD8⁺ T cell clones resulted in the elimination of HIV-1-infected cells. Target autologous, or HLA-mismatched CD4⁺ T cells were synchronously infected with the NL4-3/Ba-L chimeric clone of HIV-1 81 A (81A is congenic with NL4-3 with the exception of the V1-V3 region of env which was derived from Ba-L and confers CCR5 tropism) by magnetofection. Two hours post-infection, target cells were mixed 1:1 the L1-specific CD8⁺ T cell clone L1-3O, after thoroughly washing clone cells with PBMC, or maintained in parallel without addition effectors. Targets and effectors were co-cultured for 48 hours. Cells were then surface stained for CD4 and CD8 and stained intracellularly for HIV-1-Gag using a PE-conjugated mAb, clone Kc57 (Beckman Coulter). Flow cytometry analysis revealed robust infections of autologous and HLA-mismatched target cells cultured in the absence of L1-3O-KI9 (24.5% and 31.5% respectively). Dramatic reductions in frequencies of Gag⁺ target cells were observed in autologous cells co-cultured with L1-3O-KI9 and only modest reductions in frequencies of Gag⁺ target cells in HLA-mismatched controls (93.1% and 92.7% reductions for independent autologous elimination assays versus 30.5% and 31.1% reductions for independent HLA-mismatched elimination assays) (FIG. 4A). Thus the L1-p150-specific CD8⁺ T cell clone L1-3O-KI9 specifically eliminates HIV-1-infected primary CD4⁺ T cells in vitro.

Given the ability of L1-3O-KI9 to recognize HIV-1-infected cells very rapidly post infection (within 2 hours), and to eliminate HIV-1-infected cells, we hypothesized that this clone would suppress HIV-1-replication in vitro in a dose-dependent manner. Autologous and HLA mismatched CD4⁺ T cell targets were infected with 0.02 MOI HIV-1 NL4-3 using a standard infection protocol (not magnetofection, see protocols). These target cells were plated at 2×10⁴ cells/well in 96 well plates. CD8⁺ T cell effectors were taken 3 weeks following latest restimulation, washed with PBS, and added at 5-fold serial dilutions ranging from effector:target ratios of 1:1 to 1:3125. Each effector:target ratio was tested in triplicate, as were target only and effector only controls. The L1-3O-KI9 L1-p 1 50-specific CD8⁺ T cell clone, as well as a CMV-specific CD8⁺ T cell clone from the same individual, were both tested against autologous and HLA-mismatched target cells. At day 9 post-infection supernatants were assayed by p24 ELISA as a readout of HIV-1 particle production (NCI Frederick). The production of p24 from autologous target cells was potently suppressed by the L1-3O-KI9 clone, with maximal suppression observed at an effector:target ratio of 1:4 (FIG. 4B). Thus the L1-p150-specific CD8⁺ T cell clone L1-3O-KI9 potently inhibits the production of HIV-1-virus particles.

FIGS. 4A and 4B. Elimination of HIV-1-Infected Cells and Suppression of Virus Production by L1-specific CD8⁺ T Cell Clones. A. CD4⁺ T cell targets were synchronously infected with HIV-1-NL4-3 by magnetofection. The L1-specific CD8⁺ T cell clone L1-3O-KI9 was added to infected autologous and HLA-mismatched targets at a ratio of 1:1. Control infections cultures were maintained in parallel without the addition of CD8⁺ T cells. Infections were allowed to proceed for 18 hours at which point cells were surface stained with mAbs to CD4, and stained intracellularly with a mAb to HIV-1-Gag (Ki67, Beckman Coulter). Shown are flow cytometry plots of HIV-1-Gag (x-axis) by CD4 (y-axis). Clone suppressions are shown in duplicate. B. CD4⁺ T cell targets were infected with 0.02 MOI of HIV-1-NL4-3 and plated at 15,000 cells/well in a 96 well plate. Effector CD8⁺ T cell clones (L1-3O) were added at given ratios, and infections were allowed to proceed for 9 days. At this point, HIV-1-p24 levels in supernatants were tested in triplicate along with standards of known p24 concentrations. Shown are concentrations of p24 as calculated based on standard curve. Error bars represent standard deviation.

L1-Specific CD8⁺ T Cells Comprehensively Recognize Cells Infected with Diverse Isolates of HIV-1 and HIV-2.

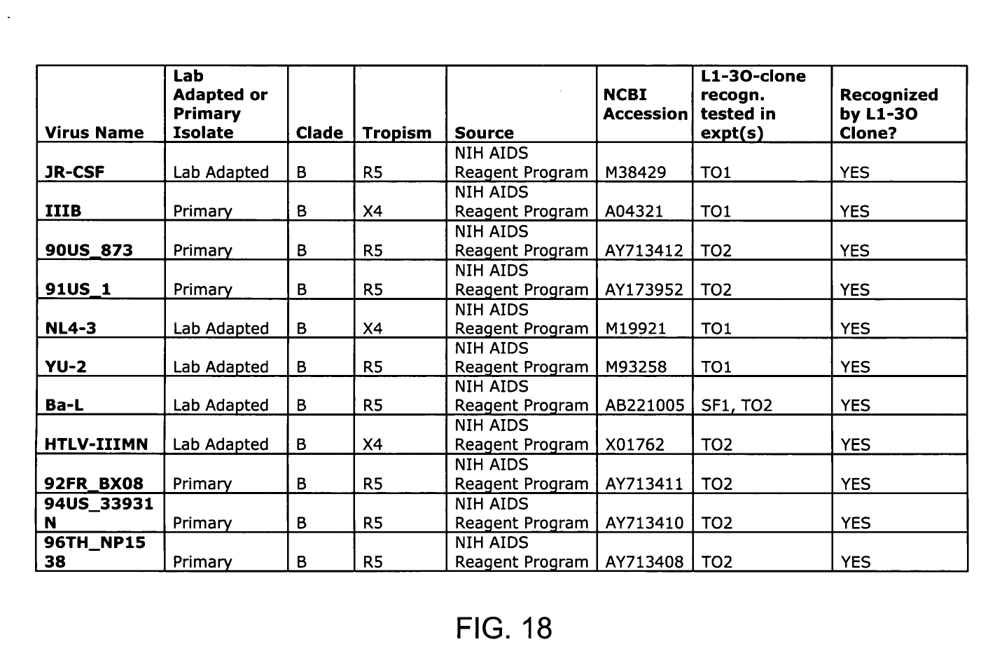

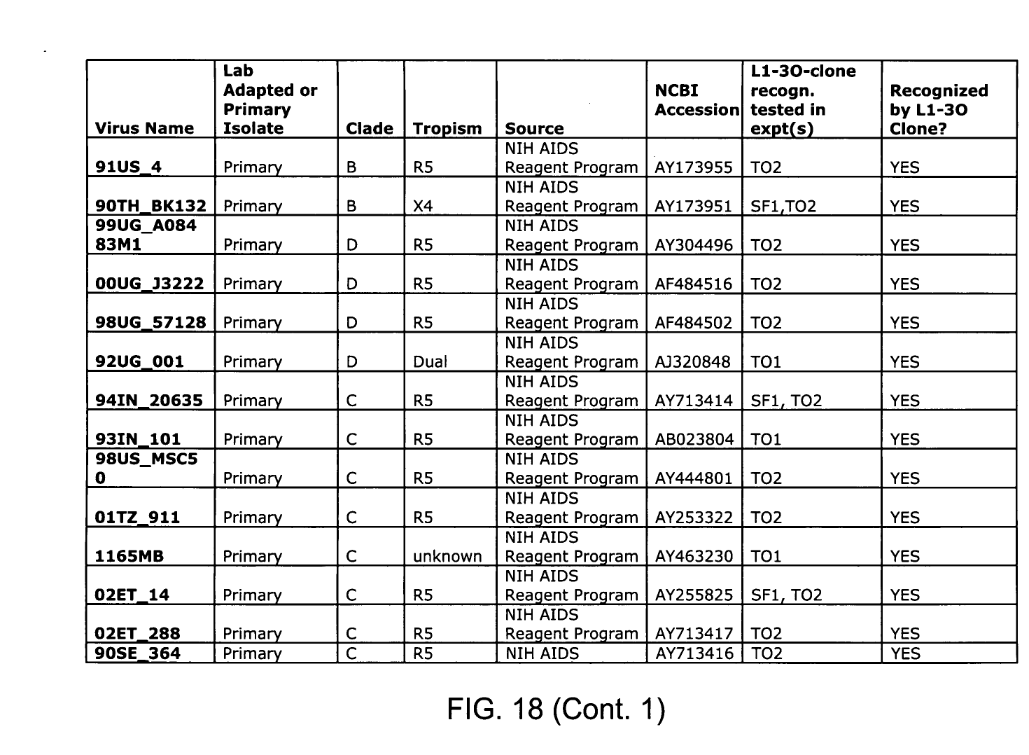

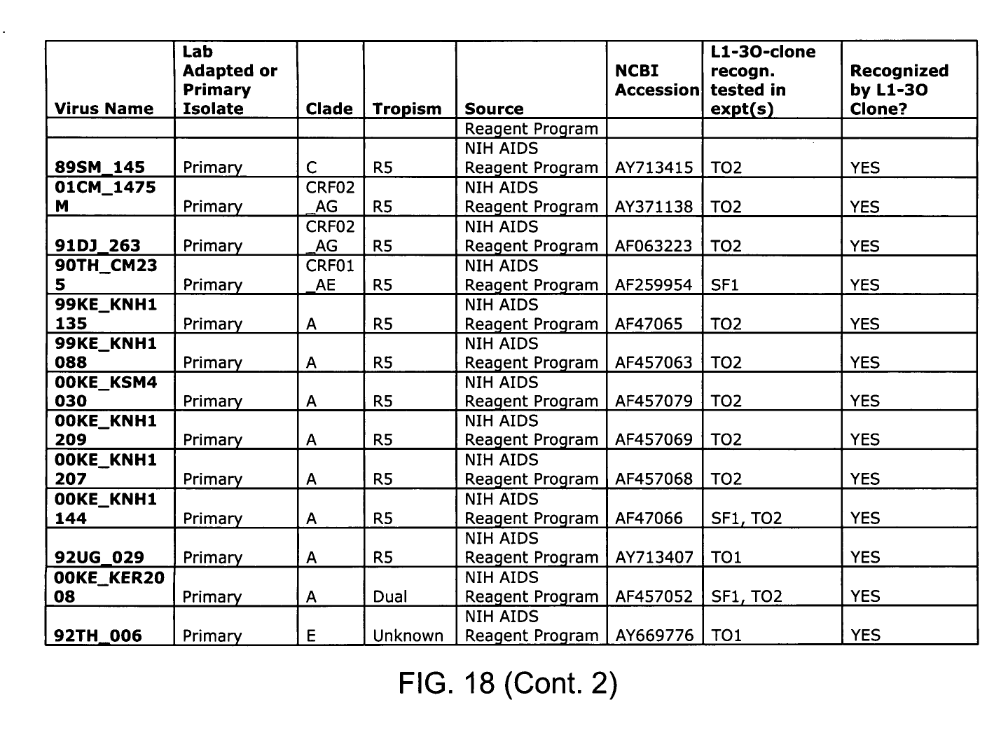

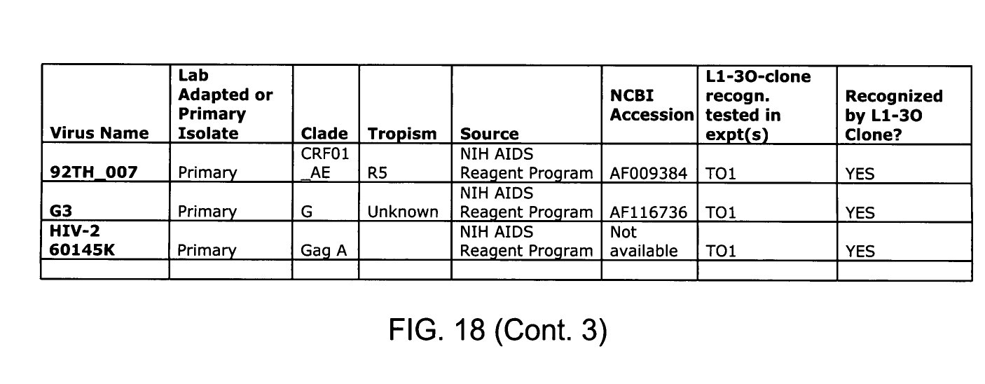

Since L1-specific T cells recognize stable genome encoded L1 antigens presented on the surface of HIV-1-infected cells, recognition of these cells should be independent of HIV-1-sequence variability. A panel of 42 diverse HIV-1 viruses comprising 5 lab adapted isolates and 37 primary isolates was acquired from the NIH AIDS reagent program. The breakdown of these primary isolate viruses by clade were: clade B—9 isolates, clade C—10 isolates, clade D—4 isolates, clade A—8 isolate, clade E—1 isolate, clade G—1 isolate, CRF01_AE—2 isolate, CRF02_AG—2 isolates. By tropism the panel comprises 4 CXCR4-tropic viruses, 27 CCR5-tropic viruses, 2 dual-tropic viruses, and 4 viruses of unknown tropism. Details of these isolates, including NCBI accession numbers, are presented in the table presented in FIG. 18. In addition to this diverse panel of HIV-1, an isolate of HIV-2 “60145K” was obtained from the NIH AIDS reagent program.

The experiment codes in the table presented in FIG. 18 are as follows: TO1: first diverse isolate recognition assay (performed in Toronto, Canada); SF1: diverse isolate recognition assay (performed in San Francisco, Calif.); TO2: diverse isolate recognition assay (performed in Toronto, Canada).

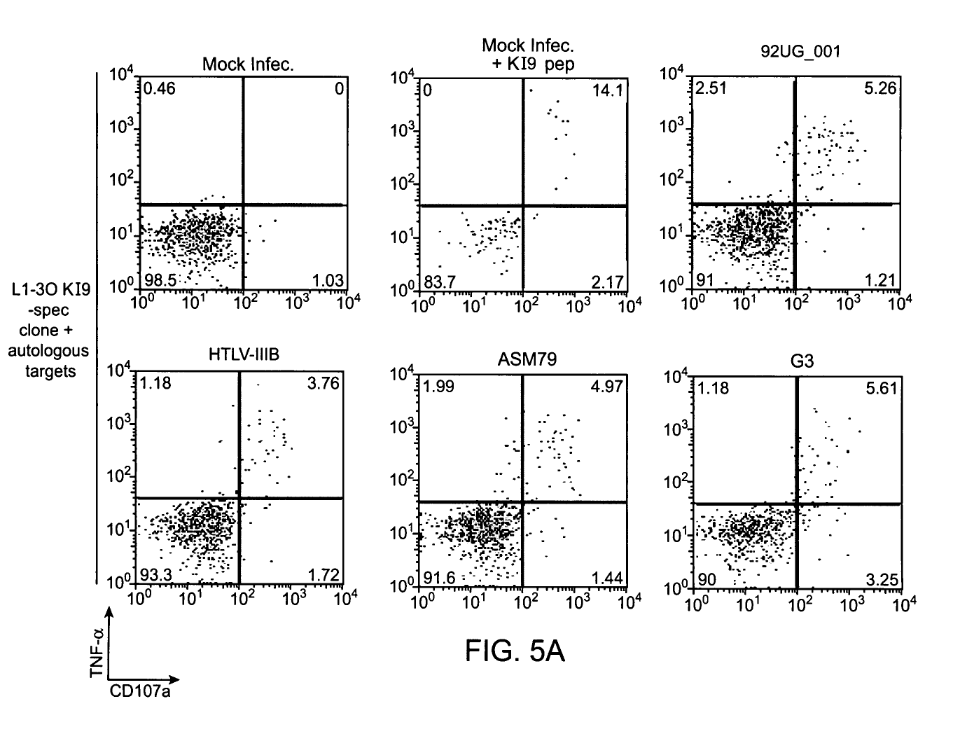

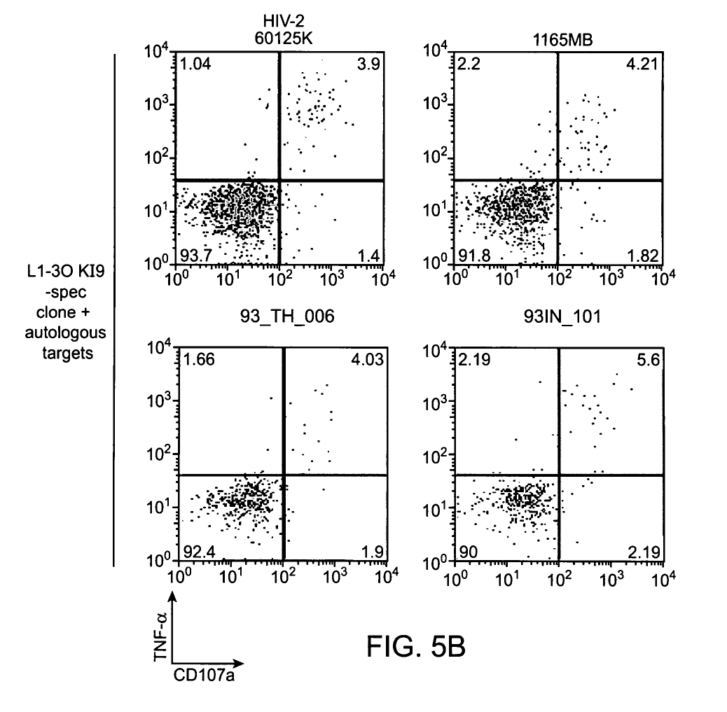

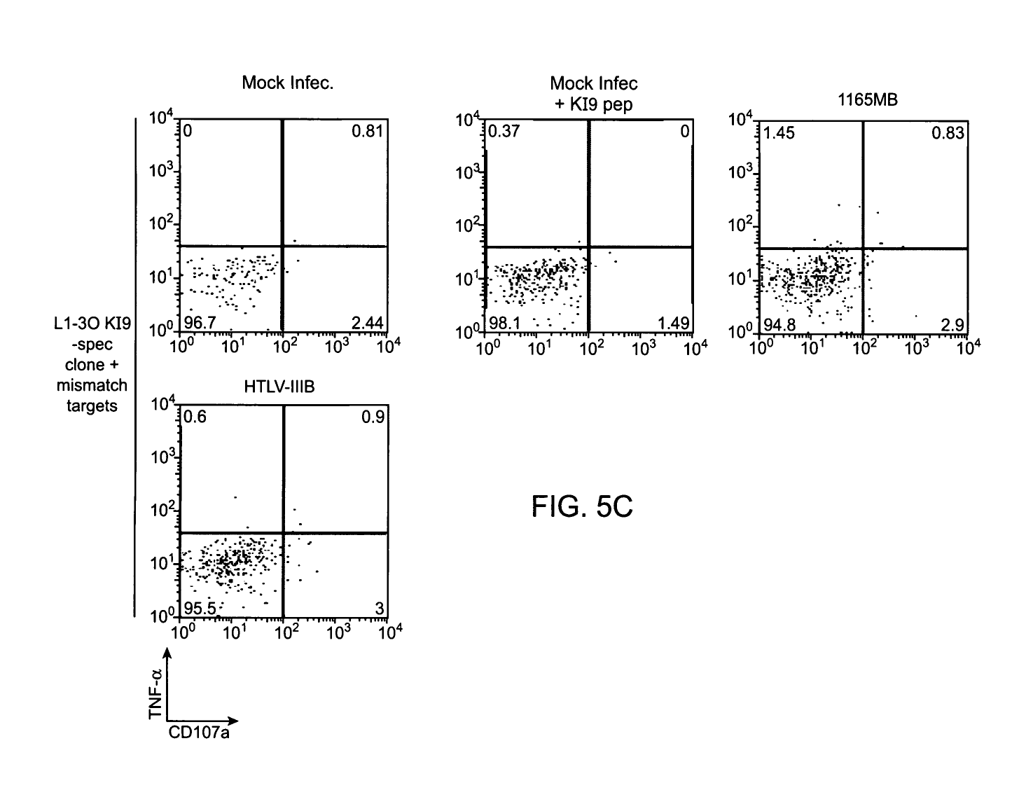

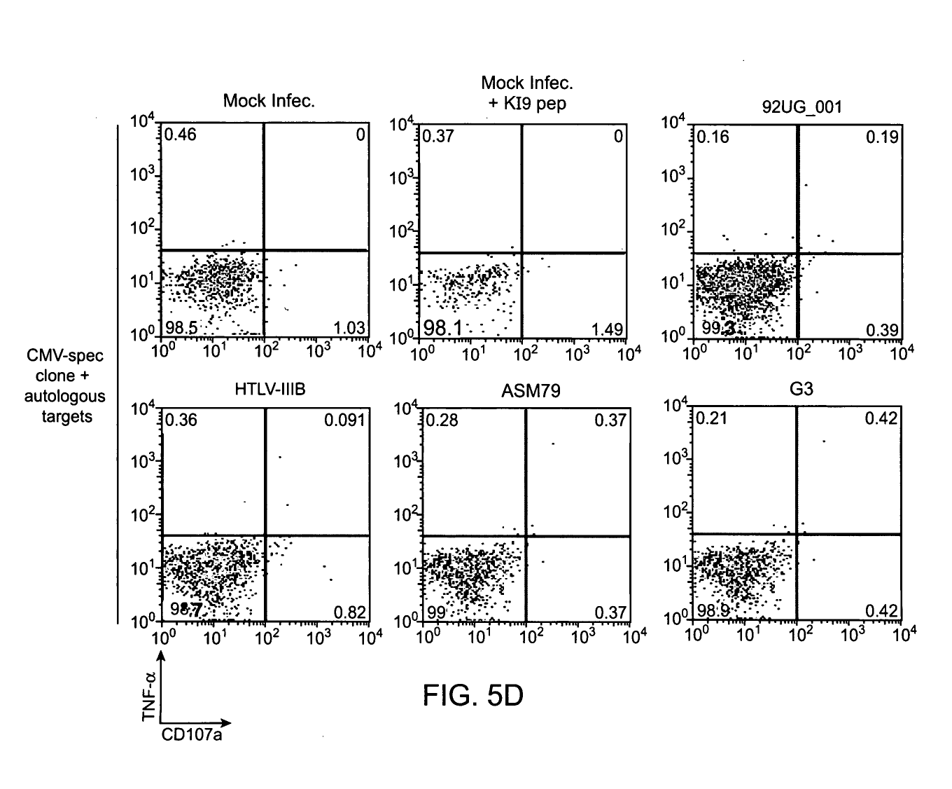

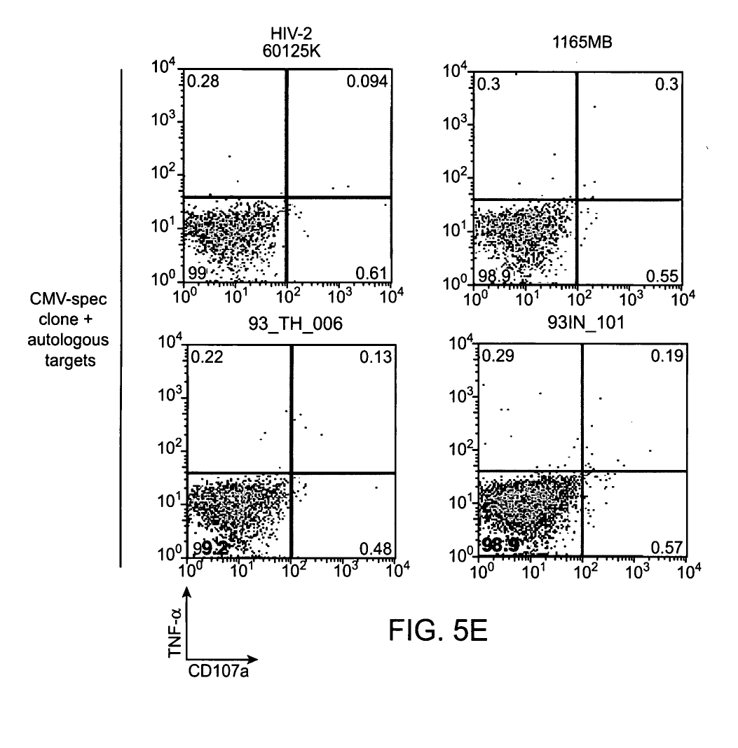

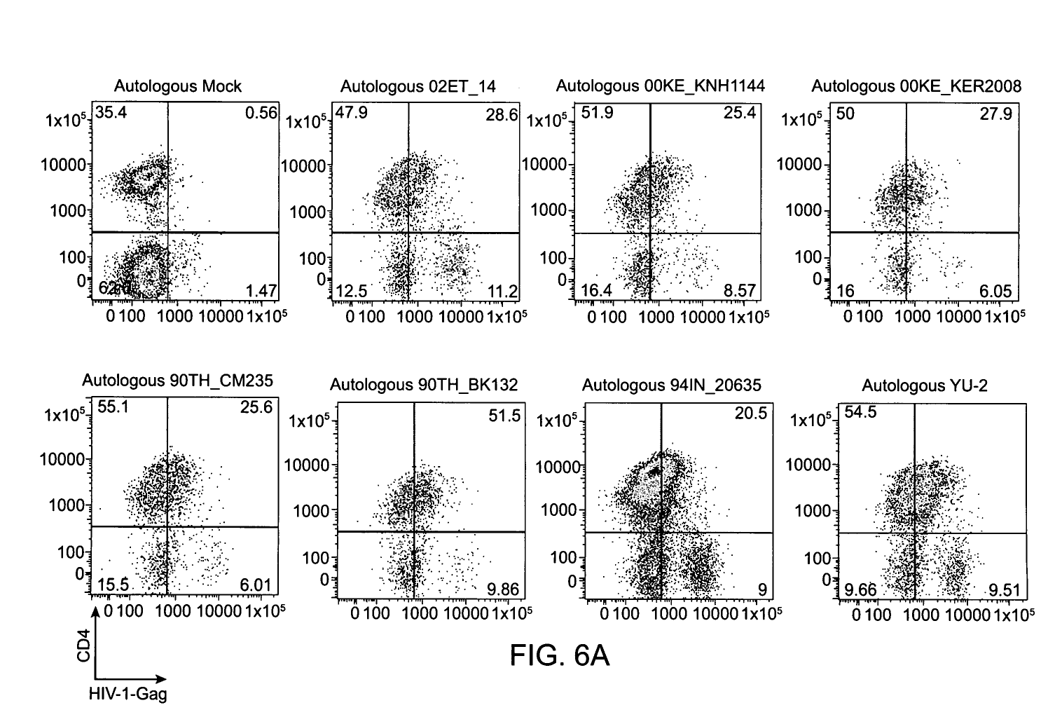

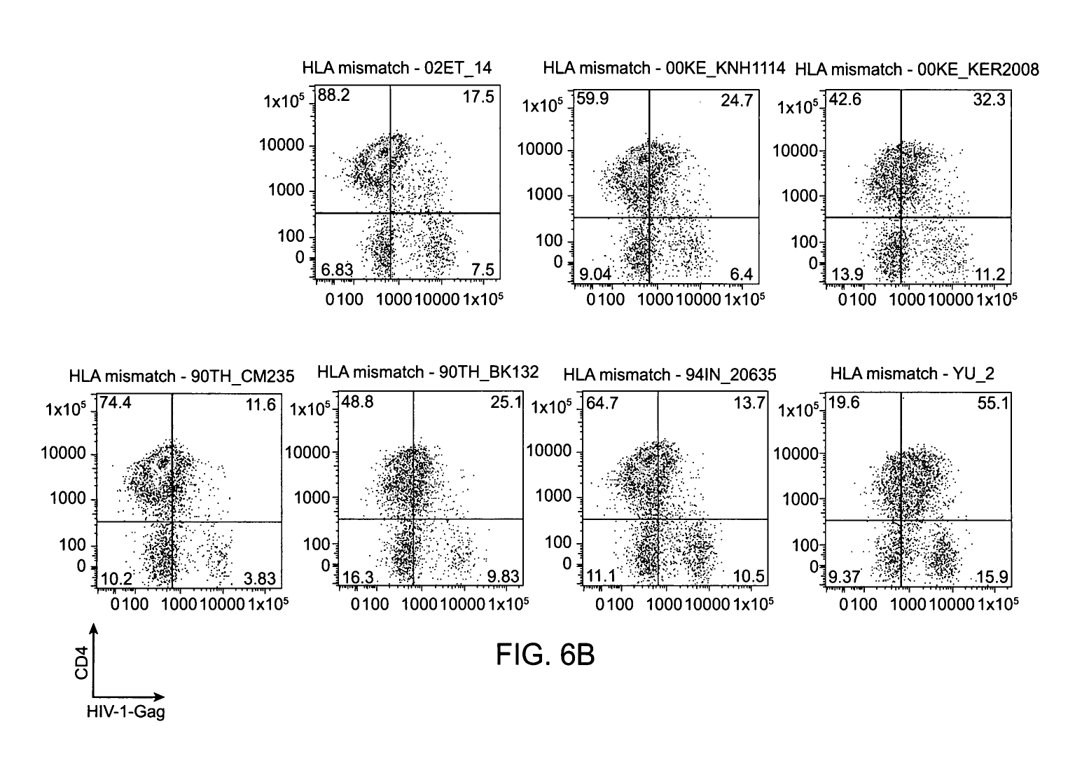

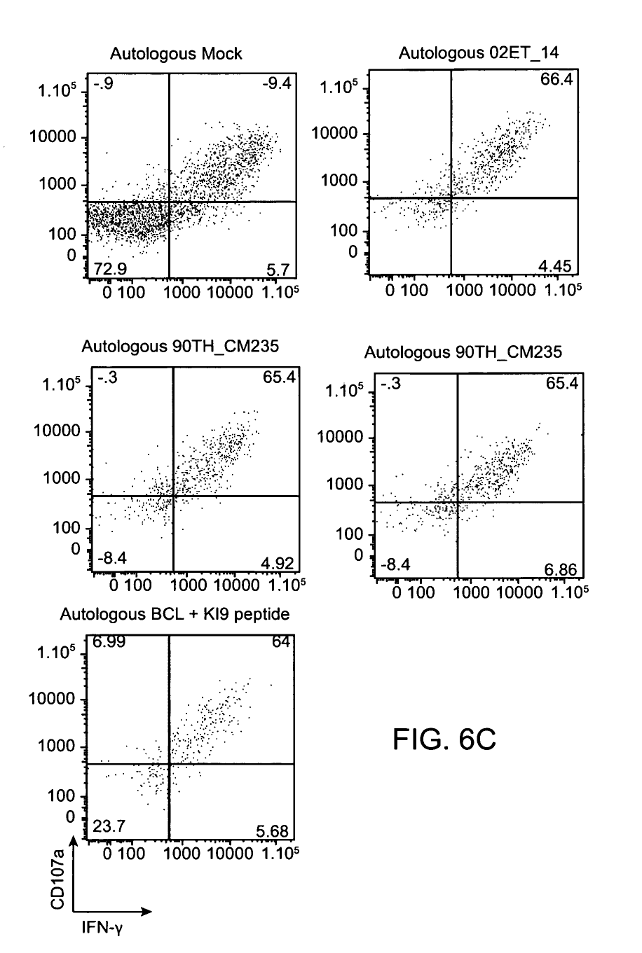

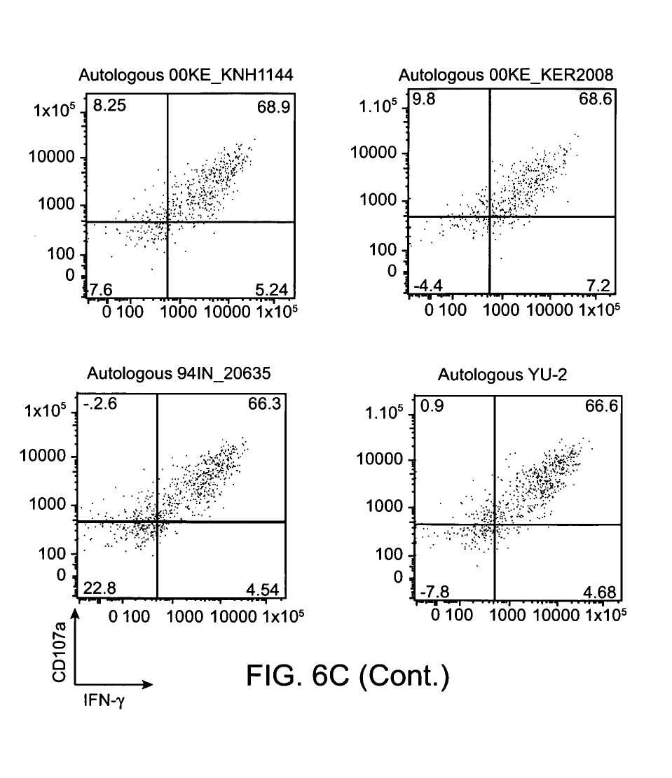

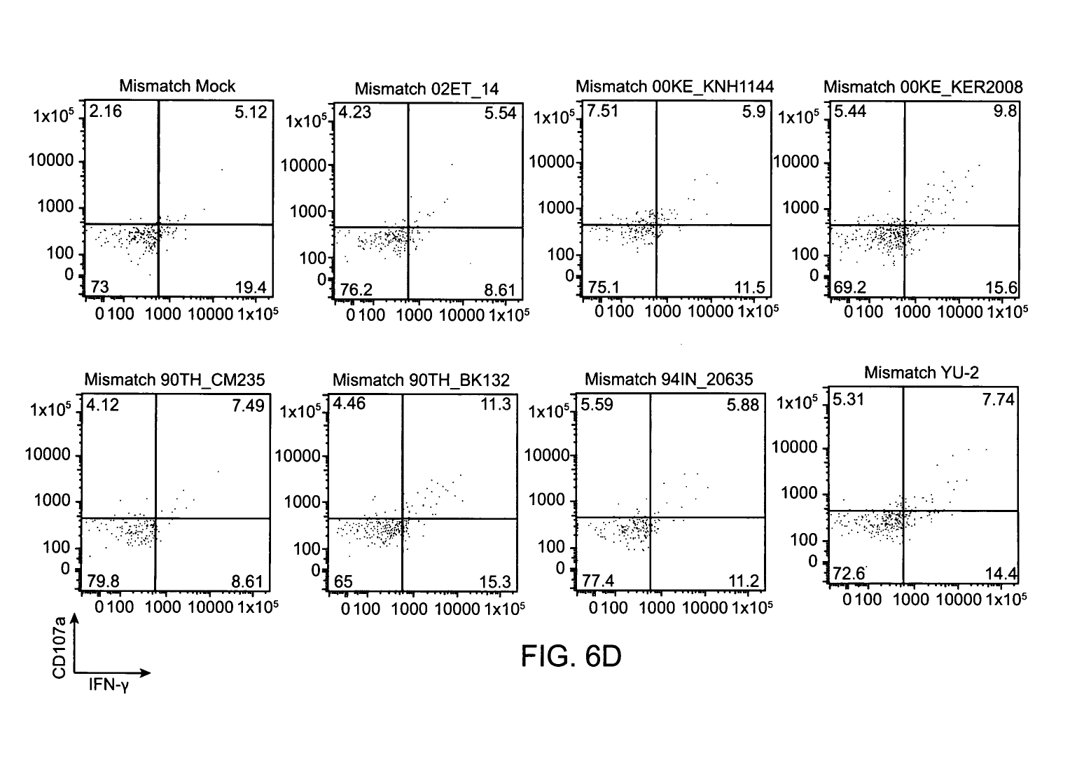

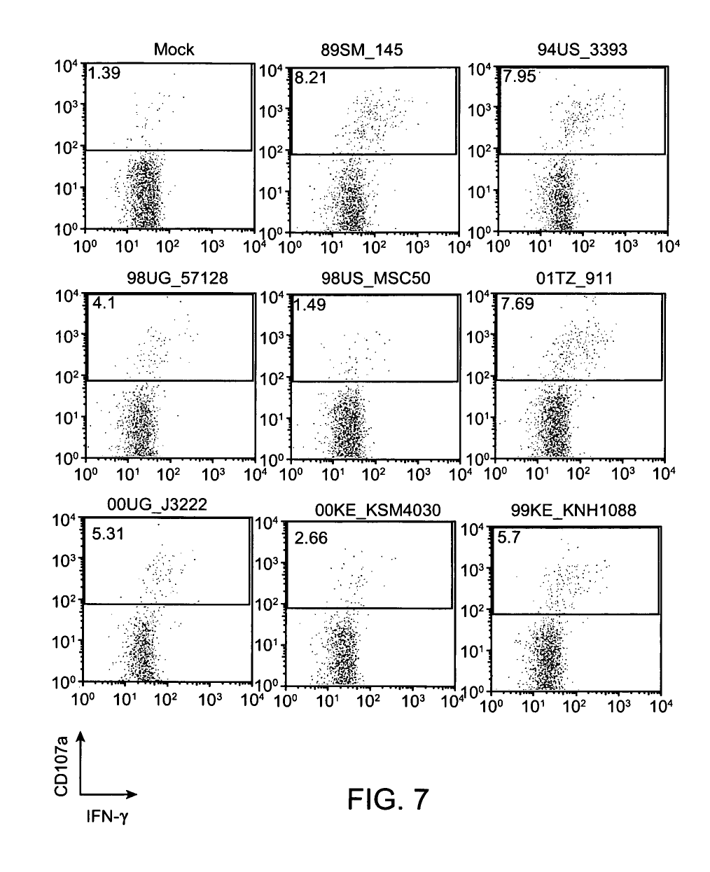

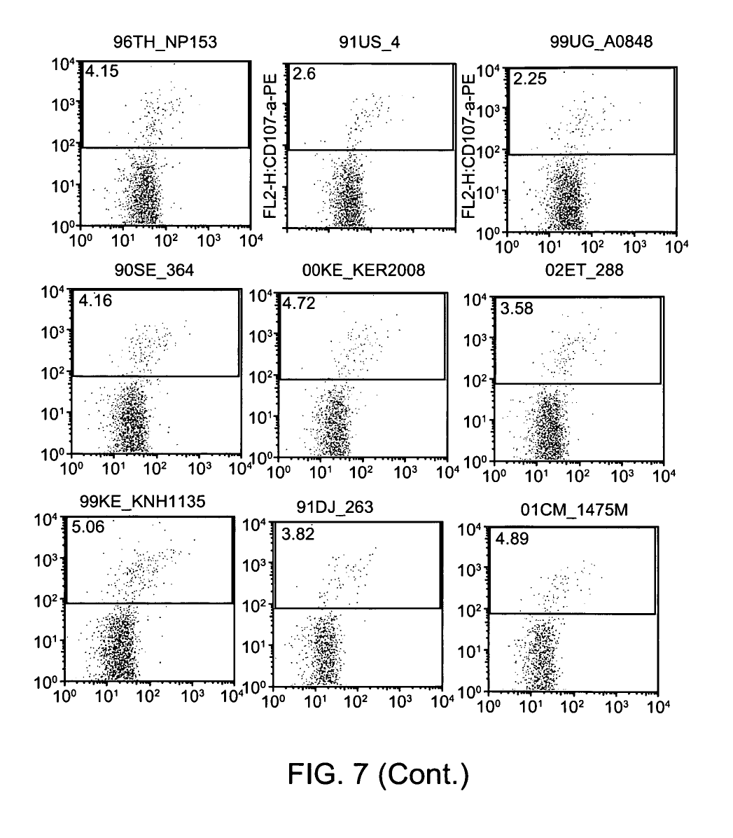

The L1-p150-specific clone L1-3O-KI9 was tested for recognition of autologous and HLA mismatched primary CD4⁺ T cells using the flow cytometry assay described above. The panel was tested in 3 separate experiments: “TO1”, “SF1”, and “TO2”. The dates of these experiments and viruses tested in each are given in the table presented in FIG. 18. Data from TO1 are displayed in FIG. 5, SF1 in FIG. 6, and TO2 in FIG. 7. In each experiment, a mock infection control was employed as a measure of the background responsiveness of clone L1-3O to uninfected autologous CD4⁺ T cells. Differing sets of additional controls were incorporated into each experiment, and differing readouts for responsiveness were used. In TO1 (FIG. 5) CD107a staining (degranulation) and TNF-α production were used as readouts for recognition.

Recognition of autologous targets infected with each of the HIV-1-isolates tested was observed, as well as recognition of HIV-2-infected autologous targets. HLA-mismatched CD4⁺ T cells were infected with HIV-1-1165MB and HIV-1-IIIB and the L1-3O clone was tested for recognition of these targets, which would indicate MHC-I-independent recognition. No recognition of these controls was observed. In parallel the ability of clone L1-3O to recognize its cognate peptide “KI9” presented either by autologous or HLA-mismatched CD4⁺ cell targets was tested. Recognition only of peptide pulsed autologous targets was observed. An additional control employed in this experiment was the testing of a CMV-pp65-specific CD8⁺ T cell clone derived from the same individual as L1-3O for recognition of HIV-1-infected autologous target cells. Lack of recognition with this CMV-pp65-specific clone was observed, further supporting the specific nature of the recognition of HIV-1-infected cells by the L1-p150-specific clone L1-3O (FIG. 5).

FIGS. 5A-E. L1-Specific T Cell Clone Recognition Assay with Diverse HIV-1 Panel and HIV-2. The recognition assay detailed in FIG. 3 was repeated with clone L1-3O using primary CD4⁺ T cells infected with a diverse panel of HIV-1 isolates, as well as with HIV-2 60145K (see the table presented in FIG. 18 for virus details). Shown is flow cytometry data gated on CD8⁺ cells (clone) plotting CD107a as a marker of degranulation by TNF-α. FIGS. 5A and 5B depict the response of the clone to autologous cells infected with the given viruses (mock=uninfected, mock+KI9 peptide=uninfected cells pulsed with 10 μg/ml of synthetic KI9 peptide). HLA-mismatched CD4⁺ T cell targets were infected with a subset of these viruses and tested as controls. The results, shown in FIG. 5C, demonstrate that clone L1-3O recognized autologous but not HLA-mismatched infected target cells. A CMV-pp65-specific CD8⁺ T cell clone obtained from the same individual as the L1-p150-specific clone L1-3O was also tested for recognition of autologous infected targets and demonstrated a lack of recognition (FIGS. 5D and E).

In experiment (FIG. 6A-D) SF1 CD107a staining (degranulation) and IFN-γ production were used as readouts for recognition. Both autologous and HLA-mismatched CD4⁺ cell targets were infected with each of the 7 viruses tested. Infections of comparable magnitude were observed in autologous and HLA-mismatched target cells for each of the viruses tested (FIGS. 6A,B). Clone L1-3O was tested for recognition of each of these targets. Recognition of autologous CD4⁺ cell targets infected with each of these viruses was observed, contrasted by a lack of recognition of HLA-mismatched target infected cells.

FIG. 6. L1-Specific T Cell Clone Diverse HIV-1 Panel Recognition Assay “SF1”. The L1-p150-specific CD8⁺ T cell clone was further tested for recognition of additional diverse isolates of HIV-1. The experimental setup is analogous to that given in FIG. 5, and details of viruses are available in the table presented in FIG. 18. Shown are A,B. Flow cytometry data showing HIV-1-Gag staining by CD4 staining as assessments of levels of HIV-1 infection in A. autologous and B. HLA-mismatched CD4⁺ T cell targets. C,D. Flow cytometry data, gated on CD8⁺ T cells (clone), showing IFN-γ staining by CD107a staining as an assessment of the responsiveness of clone cells to C. autologous and D. HLA-mismatched targets (“mock”=uninfected cells, BCL+KI9 peptide=autologous B cell line pulsed with 10 μg/ml of KI9 peptide).

In experiment TO2, CD107a staining (degranulation) and IFN-γ production were used as readouts for recognition, and tested clone L1-3O for recognition of autologous CD4⁺ target cells infected with an additional 17 HIV-1-isolates (FIG. 7). Target cells were infected with lower levels of HIV-1 than in SF1, resulting in lower levels of recognition. Some variability in the level of recognition of target cells infected with different isolates (from 1.49%-8.21% CD 107a⁺) was observed. It was tested whether the levels of responses exhibited by the clone correlated with levels of infection within CD4⁺ T cell target populations. The % of infected target cells was measured by flow cytometry staining with anti-HIV-1-Gag (Kc57-RD1, BD Biosciences) and plotted against % CD107a⁺ clone cells.

FIG. 7. L1-Specific T Cell Clone Diverse HIV-1 Panel Recognition Assay “TO2”. The L1-p150-specific CD8⁺ T cell clone was further tested for recognition of additional diverse isolates of HIV-1. The experimental setup is analogous to that given in FIGS. 5,6, and details of viruses are available in the table presented in FIG. 18.

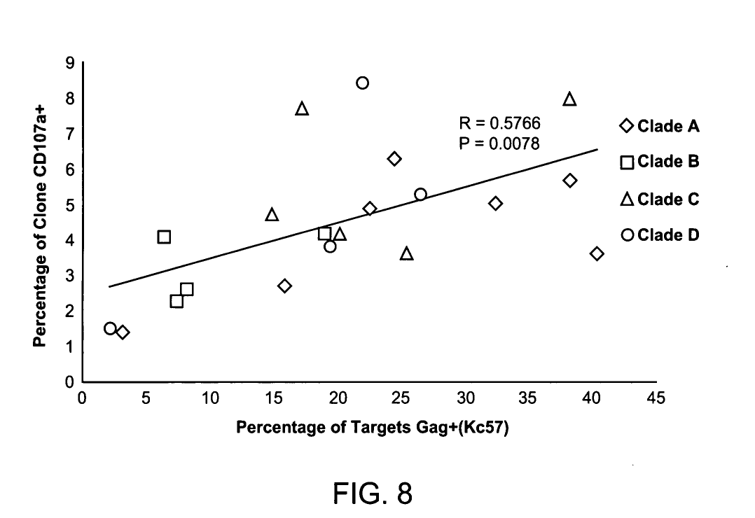

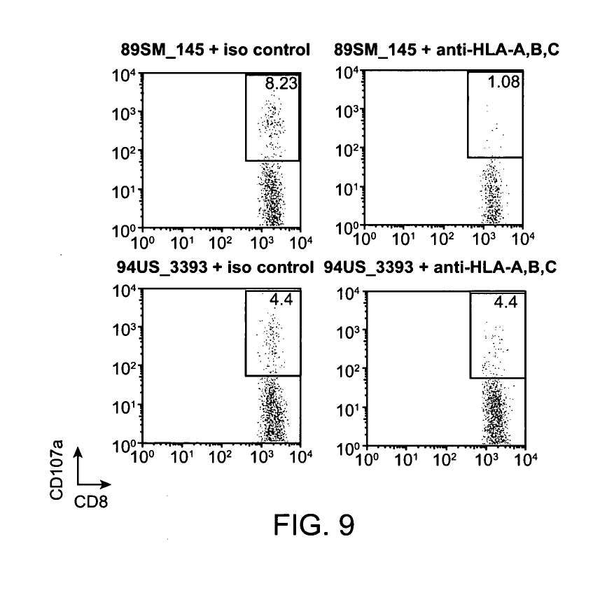

A strong correlation was observed between these two parameters (R=0.5766, p=0.0078, FIG. 8), indicating that differing levels of HIV-1-infection with these different virus stocks were a primary contributor to the variability in levels of clone responsiveness towards target cells. In addition, it was observed that the degree of recognition per infected target cell was not consistently higher or lower for any particular clade of virus (FIG. 8). As variations in viral sequence cluster with clades, this provides evidence that sequence variability in HIV-1 isolates is not a factor in the degree of recognition observed with the L1-p150-specific clone L1-3O. As an additional control in experiment TO2, it was tested whether pre-incubation of target cells infected with either HIV-1-89SM_{—}145 or HIV-1-94US_{—}3393 with 10 μg/ml of a blocking antibody to HLA-A,B,C (clone G46-2.6, BD Biosciences) would prevent recognition by clone L1-3O. This was compared to pre-incubation of infected target cells with an equal amount of IgG1 isotype control. A potent inhibition of recognition with HLA-A,B,C pre-incubation was observed (FIG. 9). Taken together, these data demonstrate that the L1-p150-KI9-specific CD8⁺ T cell clone “L1-3O” recognizes cells infected with HIV-1 or HIV-2, independent of sequence variability, in an MHC-I restricted manner.

FIG. 8. The Degree of Responsiveness of Clone L1-3O to Autologous Cells Infected with Diverse HIV-1-Isolates Correlates with the Level of Infection. The percentage of HIV-1-infected cells in target populations used for the recognition assay depicted in FIG. 7 were measured by flow cytometry, staining with a fluorochrome antibody to HIV-1-Gag (Kc57-RD1). Shown is the percentage of infected targets(x-axis) by the percentage of clone L1-3O responding by degranulating (CD 107a+).

FIG. 9. Blockade of Infected Cell Recognition by Pre-Incubation with Anti-HLA-A,B,C antibody. In the recognition assay depicted in FIG. 7 recognition of the viruses 89SM_{—}145, and 94 US_{—}3393 was also tested after autologous target cells were pre-incubated with 10 μg/ml of anti-HLA-A,B,C antibody (clone G46-2.6, BD Biosciences) or 10 μg/ml of IgG1 isotype control. Shown is flow cytometry data depicting CD8 staining by CD107a staining (as a marker of degranulation).

### Example 3

### L1-Specific T Cell Clones Mediate Killing of Cells Infected with Diverse Isolates of HIV-1

The L1-ORF-2-KVIYRFNAI (SEQ ID NO:10) epitope-specific clone ‘L1-3O’ was tested for killing of HIV-infected cells.

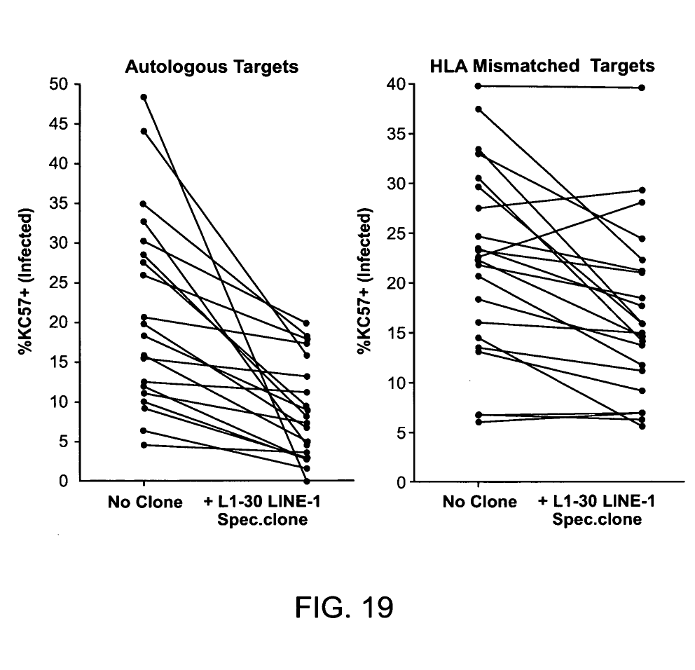

CD4⁺ T cell targets from two donors, the individual from whom the L1-3O clone was derived and an HLA-mismatched individual, were synchronously infected with a panel of diverse isolates of HIV-1 by magnetofection, and infections were allowed to proceed for 24 hours. The L1-specific CD8⁺ T cell clone L1-3O-KI9 was added to infected autologous and HLA-mismatched targets at a ratio of 1:15 clone:target. Control infection cultures were maintained in parallel without the addition of CD8⁺ T cells. Infections were allowed to proceed for an additional 18 hours at which point cells were surface stained with mAbs to CD4, and stained intracellularly with a mAb to HIV-1-Gag (Ki67, Beckman Coulter). Degree of infection was measured as % of cells staining Ki67+ in a flow cytometry assay. The data are shown in FIG. 19.

As shown in FIG. 19, the LINE-1-ORF2p-specific clone L1-3O-KI9 potently eliminated autologous cells infected with every isolate of HIV-1 tested. The reductions of infection levels in HLA-mismatched targets co-cultured with clone were lesser in magnitude than that observed in autologous targets. The data demonstrate that LINE-1-specific CD8⁺ T cells specifically recognize and eliminate autologous cell infected with diverse isolates of HIV1.

### Example 4

### L1-Specific T Cell Clones Mediate Killing of HIV-Infected Cells

CD8⁺ T cells were isolated from an HIV-infected individual exhibiting natural control of infection (HIV viral load<50 copies/ml bDNA) without therapy. These cells were expanded in vitro for 7 days with either: LINE-1-ORF1-RPNLRLIGV (SEQ ID NO:9), HIV-Gag pooled peptides (consensus peptides from NIH AIDS Reagent Program), or CMV pp65 pooled peptides (JPT Peptide Technologies) using peptide pulsed autologous B cell lymphoma lines. These expanded B cell lines were mixed in a 1:1 ratio with autologous CD4⁺ T cells which had either been infected with HIV-1-NL4-3, or maintained as a mock infection control. As a positive control, expanded lines were also mixed with HIV-1-NL4-3 infected autologous CD4⁺ T cells which had been pulsed with peptide (either RPNLRLIGV (SEQ ID NO:9), pooled Gag peptides, or pooled CMV pp65 peptides).

Effectors and targets were cultured together at 37° C., 5% CO₂ for 1 hour. 5 μg/ml of PE-conjugated anti-CD107a mAb (BD) was added at this stage (prior to 1 hour co-culture). Brefeldin A was then added to a final concentration of 10 μg/ml and incubations were allowed to proceed for an additional 5 hours. Cells were then surface stained with mAbs to CD4 and CD8, permeabilized using cytofix/cytoperm (Becton Dickinson; BD), and then stained for either interferon-gamma (IFN-γ) or tumor necrosis factor-alpha (TNF-α) using mAbs (BD). Flow cytometry was performed on a FACSCalibur instrument (BD).

The results indicate that HIV-1-NL4-3 infected autologous cells stimulate LINE-1-RPNLRLIGV (SEQ ID NO:9) expanded CD8+ T cells, as well as HIV-1-Gag expanded CD8⁺ T cells. HIV-1-NL4-3 infected autologous cells did not stimulate CMV-pp65 expanded CD8⁺ T cells (employed as a negative control). The data demonstrate that LINE-1-specific CD8⁺ T cells specifically recognize HIV-1-infected cells.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. An immunogenic composition comprising an isolated long interspersed nuclear element (LINE) polypeptide and a pharmaceutically acceptable carrier.

2. The immunogenic composition of claim 1, wherein the isolated LINE polypeptide comprises an amino acid sequence having at least about 75% amino acid sequence identity to any one of SEQ ID NOs:1-22.

3. The immunogenic composition of claim 1, wherein the isolated LINE polypeptide comprises an amino acid sequence set forth in any one of SEQ ID NOs:1-22.

4.-5. (canceled)

6. The immunogenic composition of claim 1, further comprising an adjuvant.

7. The immunogenic composition of claim 6, wherein the adjuvant comprises aluminum hydroxide, MF59, or monophosphoryl lipidA.

8. An immunogenic composition comprising a nucleic acid comprising a nucleotide sequence encoding a long interspersed nuclear element (LINE) polypeptide.

9. The immunogenic composition of claim 8, wherein the LINE polypeptide comprises an amino acid sequence as set forth in any one of SEQ ID NOs:1-22.

10.-11. (canceled)

12. The immunogenic composition of claim 8, wherein the nucleic acid is a recombinant vector.

13. The immunogenic composition of claim 12, wherein the recombinant vector is a recombinant viral vector.

14. A synthetic long interspersed nuclear element (LINE) polypeptide comprising an amino acid sequence having at least about 75% amino acid sequence identity to the amino acid sequence set forth in one of SEQ ID NOs:1-22.

15. (canceled)

16. The synthetic LINE polypeptide of claim 14, wherein the polypeptide is multimerized.

17. The synthetic LINE polypeptide of claim 14, wherein the polypeptide is linked to a carrier.

18.-21. (canceled)

22. A nucleic acid comprising a nucleotide sequence encoding the synthetic LINE polypeptide of claim 14.

23. (canceled)

24. A method of inducing a T lymphocyte response in an individual to a host cell infected with or at risk of infection with a pathogenic virus, the method comprising administering to the individual a composition comprising a LINE polypeptide or composition comprising a nucleic acid that comprises a nucleotide sequence encoding a LINE polypeptide.

25. The method of claim 24, wherein the T lymphocyte response comprises a CD8⁺ T cell response or a CD4⁺ T cell response.

26. The method of claim 24, wherein the T lymphocyte response comprises a mucosal T lymphocyte response.

27. The method of claim 24, wherein the pathogenic virus is a human immunodeficiency virus.

28. The method of claim 24, wherein the individual has not been infected with the pathogenic virus.

29. The method of claim 24, wherein the individual has been infected with the pathogenic virus.

30.-31. (canceled)

32. A method of treating a retrovirus infection in an individual, the method comprising administering to the individual an effective amount of a composition comprising a LINE polypeptide or composition comprising a nucleic acid that comprises a nucleotide sequence encoding a LINE polypeptide.

33. The method of claim 32, wherein said administering is effective to reduce viral load in the individual by at least about 10%.

34. The method of claim 32, wherein the retrovirus is a human immunodeficiency virus (HIV).

35. The method of claim 34, further comprising administering to the individual an effective amount of one or more of a nucleotide analog reverse transcriptase inhibitor, a nucleoside analog reverse transcriptase inhibitor, a non-nucleoside analog reverse transcriptase inhibitor, an HIV protease inhibitor, an HIV integrase inhibitor, and an HIV entry/fusion inhibitor.

36.-38. (canceled)

39. A method of monitoring a patient's response to a treatment for a retrovirus infection, the method comprising:
a) contacting a white blood cell (WBC) in vitro with synthetic long interspersed nuclear element (LINE) polypeptide, wherein the WBC is obtained from the patient at a first time point following the beginning of the treatment; and
b) detecting a cytokine secreted by the WBC in response to contact with the LINE polypeptide,
wherein a reduction in cytokine production by the WBC in response to contact with a LINE polypeptide, compared to the level of cytokine production by a control WBC in response to contact with the LINE polypeptide, indicates that the treatment is effective in treating the retrovirus infection, wherein the control WBC is obtained from the patient before the start of treatment, or at a time point during the treatment that is earlier than the first time point.


SEQUENCE LISTING (499 sequences)

SEQ ID NO: 1 - protein, 12 aa, Artificial Sequence
  Description of Artificial Sequence Synthetic peptide
MNEMKREGKFRE

SEQ ID NO: 2 - protein, 10 aa, Artificial Sequence
  Description of Artificial Sequence Synthetic peptide
SQLKELEKQE

SEQ ID NO: 3 - protein, 12 aa, Artificial Sequence
  Description of Artificial Sequence Synthetic peptide
MLRAAREKGWVT

SEQ ID NO: 4 - protein, 10 aa, Artificial Sequence
  Description of Artificial Sequence Synthetic peptide
KIDRLLARLI

SEQ ID NO: 5 - protein, 9 aa, Artificial Sequence
  Description of Artificial Sequence Synthetic peptide
LRAAREKGC

SEQ ID NO: 6 - protein, 13 aa, Artificial Sequence
  Description of Artificial Sequence Synthetic peptide
NGKQKKAGFAILV

SEQ ID NO: 7 - protein, 9 aa, Artificial Sequence
  Description of Artificial Sequence Synthetic peptide
DELREEGVR

SEQ ID NO: 8 - protein, 9 aa, Artificial Sequence
  Description of Artificial Sequence Synthetic peptide
TMRYHLTPV

SEQ ID NO: 9 - protein, 9 aa, Artificial Sequence
  Description of Artificial Sequence Synthetic peptide
RPNLRLIGV

SEQ ID NO: 10 - protein, 9 aa, Artificial Sequence
  Description of Artificial Sequence Synthetic peptide
KVIYRFNAI

SEQ ID NO: 11 - protein, 9 aa, Artificial Sequence
  Description of Artificial Sequence Synthetic peptide
IVYLENPIV

SEQ ID NO: 12 - protein, 9 aa, Artificial Sequence
  Description of Artificial Sequence Synthetic peptide
SLQEIWDYV

SEQ ID NO: 13 - protein, 9 aa, Artificial Sequence
  Description of Artificial Sequence Synthetic peptide
NLEECITRI

SEQ ID NO: 14 - protein, 9 aa, Artificial Sequence
  Description of Artificial Sequence Synthetic peptide
TPRHIIVRF

SEQ ID NO: 15 - protein, 9 aa, Artificial Sequence
  Description of Artificial Sequence Synthetic peptide
LLFNIVLEV

SEQ ID NO: 16 - protein, 9 aa, Artificial Sequence
  Description of Artificial Sequence Synthetic peptide
YTMEYYAAI

SEQ ID NO: 17 - protein, 9 aa, Artificial Sequence
  Description of Artificial Sequence Synthetic peptide
RARIAKSIL

SEQ ID NO: 18 - protein, 9 aa, Artificial Sequence
  Description of Artificial Sequence Synthetic peptide
APRFIKQVL

SEQ ID NO: 19 - protein, 9 aa, Artificial Sequence
  Description of Artificial Sequence Synthetic peptide
ISYPAKLSF

SEQ ID NO: 20 - protein, 9 aa, Artificial Sequence
  Description of Artificial Sequence Synthetic peptide
SSPATEQSW

SEQ ID NO: 21 - protein, 9 aa, Artificial Sequence
  Description of Artificial Sequence Synthetic peptide
KATVTKTAW

SEQ ID NO: 22 - protein, 9 aa, Artificial Sequence
  Description of Artificial Sequence Synthetic peptide
RVNRQPTTW

SEQ ID NO: 23 - protein, 1275 aa, Homo sapiens
MTGSNSHITILTLNINGLNSAIKRHRRASWIKSQDPSVCCIQETHLTCRDTHRLKIKGWRKIYQANGKQKKAGVAILVSDKTDFKPTKIKRDKEGHYIMVKGSIQQEELTILNIYAPNTGAPRFIKQVLSDLQRDLDSHTLIMGDFNTPLSTLDRSTRQKVNKDTQELNSALHQADLIDIYRTLHPKSTEYTFFLAPHHTYSKIDHIVGSKALLSKCKRTEIITNYLSDHSAIKLELRIKNLTQSRSTTWKLNNLLLNDYWVHNEMKAEIKMFFETNENKDTTYQNLWDAFKAVCRGKFIALNAYKRKQERSKIDTLTSQLKELEKQEQTHSKASRRQEITKIRAELKEIETQKTLQKINESRSWFFERINKIDRPLARLIKKKREKNQIDTIKNDKGDITTDPTEIQTTIREYYKHLYANKLENLEEMDTFLDTYTLPRLNQEEVESLNRPITGSEIVAIINSLPTKKSPGPDGFTAEFYQRYKEELVPFLLKLFQSIEKEGILPNSFYEASIILIPKPGRDTTKKENFRPISLMNIDAKILNKILANRIQQHIKKLIHHDQVGFIPGMQGWFNIRKSINVIQHINRAKDKNHMIISIDAEKAFDKIQQPFMLKTLNKLGIDGTYFKIIRAIYDKPTANIRLNGQKLEAFPLKTGTRQGCPLSPLLFNIVLEVLARAIRQEKEIKGIQLGKEEVKLSLFADDMIVYLENPIVSAQNLLKLISNFSKVSGYKINVQKSQAFLYTNNRQTESQIMSELPFTIASKRIKYLGIQLTRDVKDLFKENYKPLLKEIKEDTNKWKNIPCSWVGRINIVKMAILPKVIYRFNAIPIKLPMTFFTELEKTTLKFIWNQKRACIAKSILSQKNKAGGITLPDFKLYYKATVTKTAWYWYQNRDIDQWNRTEPSEITPHIYNYLIFDKPEKNKQWGKDSLFNKWCWENWLAICRKLKLDPFLTPYTKINSRWIKDLNVRPKTIKTLEENLGITIQDIGTGKDFMSKTPKAMATKAKIDKWDLIKLKSFCTAKETTIRVNRQPTTWEKIFATYSSDKGLISRIYNELKQIYKKKTNNPIKNWAKDMNRHFSKEDIYAAKKHMKKCSSSLAIREMQIKTTMRYHLTPVRMAIIKKSGNNRCWRGCGEIGTLLHCWWDCKLVQPLWKSVWRFLRDLELEIPFDPAIPLLGIYPNDYKSCCYKDTCTRMFIAALFTIAKTWNQPKCPTMIDWIKKMWHIYTMEYYAAIKNDEFMSFVGTWMKLETIIVSKLSQEQKTKHRIFSLIGGN

SEQ ID NO: 24 - protein, 338 aa, Homo sapiens [sequence omitted]

SEQ ID NO: 25 - protein, 1275 aa, Homo sapiens [sequence omitted]

SEQ ID NO: 26 - protein, 1275 aa, Homo sapiens [sequence omitted]

SEQ ID NO: 27 - protein, 338 aa, Homo sapiens [sequence omitted]

SEQ ID NO: 28 - protein, 338 aa, Homo sapiens [sequence omitted]

SEQ ID NO: 29 - protein, 1275 aa, Homo sapiens [sequence omitted]

SEQ ID NO: 30 - protein, 338 aa, Homo sapiens [sequence omitted]

SEQ ID NO: 31 - protein, 1275 aa, Homo sapiens [sequence omitted]

SEQ ID NO: 32 - protein, 338 aa, Homo sapiens [sequence omitted]

SEQ ID NO: 33 - protein, 1275 aa, Homo sapiens
MTGSNSHITILTLNVNGLNSPIKRHRLASWIKSQDPSVCCIQETHLTCRDTHRLKIKGWRKIYQANGKQKKAGVAILVSDKTDFKPTKIKRDKEGHYIMVKGSIQQEELTILNIYAPNTGAPRFIKQVLSDLQRDLDSHTLIMGDFNTPLSILDRSTRQKVNKDTQELNSALHQTDLIDIYRTLHPKSTEYTFFSAPHHTYSKIDHIVGSKALLSKCKRTEIITNYLSDHSAIKLELRIKNLTQSRSTTWKLNNLLLNDYWVHNEMKAEIKMFFETNENKDTTYQNLWDAFKAVCRGKFIALNAYKRKQERSKIDTLTSQLKELEKQEQTHSKASRRQEITKIRAELKEIETQKTLQKINESRSWFFERINKIDRPLARLIKKKREKNQIDTIKNDKGDITTDPTEIQTTIREYYKHLYANKLENLEEMDTFLDTYTLPRLNQEEVESLNRPITGSEIVAIINSLPTKKSPGPDGFTAEFYQRYKEELVPFLLKLFQSIEKEGILPNSFYEASIILIPKPGRDTTKKENFRPISLMNIDAKILNKILANRIQQHIKKLIHHDQVGFIPGMQGWFNIRKSINVIQHINRAKDKNHVIISIDAEKAFDKIQQPFMLKTLNKLGIDGMYLKIIRAIYDKPTANIILNGQKLEAFPLKTGTRQGCPLSPLLFNIVLEVLARAIRQEKEIKGIQLGKEEVKLSLFADDMIVYLENPIVSAQNLLKLISNFSKVSGYKINVQKSQAFLYNNNRQTESQIMGELPFTIASKRIKYLGIQLTRDVKDLFKENYKPLLKEIKEDTNKWKNIPCSWVGRINIVKMAILPKVIYRFNAIPIKLPMTFFTELEKTTLKFIWNQKRARIAKSILSQKNKAGGITLPDFKLYYKATVTKTAWYWYQNRDIDQWNRTEPSEIMPHIYNYLIFDKPEKNKQWGKDSLLNKWCWENWLAICRKLKLDPFLTPYTKINSRWIKDLNVKPKTIKTLEENLGITIQDIGVGKDFMSKTPKAMATKDKIDKWDLIKLKSFCTAKETTIRVNRQPTTWEKIFATYSSDKGLISRIYNELKQIYKKKTNNPIKKWAKDMNRHFSKEDIYAAKKHMKKCSSSLAIREMQIKTTMRYHLTPVRMAIIKKSGNNRCWRGCGEIGTLVHCWWDCKLVQPLWKSVWRFLRDLELEIPFDPAIPLLGIYPKDYKSCCYKDTCTRMFIAALFTIAKTWNQPNCPTMIDWIKKMWHIYTMEYYAAIKNDEFISFVGTWMKLETIILSKLSQEQKTKHRIFSLIGGN

SEQ ID NO: 34 - protein, 338 aa, Homo sapiens [sequence omitted]

SEQ ID NO: 35 - protein, 1275 aa, Homo sapiens [sequence omitted]

SEQ ID NO: 36 - protein, 337 aa, Homo sapiens [sequence omitted]

SEQ ID NO: 37 - protein, 338 aa, Homo sapiens [sequence omitted]

SEQ ID NO: 38 - protein, 1275 aa, Homo sapiens [sequence omitted]

SEQ ID NO: 39 - protein, 338 aa, Homo sapiens [sequence omitted]

SEQ ID NO: 40 - protein, 338 aa, Homo sapiens [sequence omitted]

SEQ ID NO: 41 - protein, 1275 aa, Homo sapiens [sequence omitted]

SEQ ID NO: 42 - protein, 7 aa, Homo sapiens
EMKREGK

SEQ ID NO: 43 - protein, 7 aa, Human immunodeficiency virus 1
EMKREGK

SEQ ID NO: 44 - protein, 8 aa, Homo sapiens
NEMKREGK

SEQ ID NO: 45 - protein, 8 aa, Human immunodeficiency virus 1
NEMEREGK

SEQ ID NO: 46 - protein, 8 aa, Human immunodeficiency virus 1
DEMKKEGK

SEQ ID NO: 47 - protein, 8 aa, Homo sapiens
QLKELEKQ

SEQ ID NO: 48 - protein, 8 aa, Human immunodeficiency virus 1
QIKELQKQ

SEQ ID NO: 49 - protein, 9 aa, Homo sapiens
SQLKELEKQ

SEQ ID NO: 50 - protein, 11 aa, Human immunodeficiency virus 1
SELQTKELQKQ

SEQ ID NO: 51 - protein, 12 aa, Homo sapiens
MLRAAREKGWVT

SEQ ID NO: 52 - protein, 14 aa, Human immunodeficiency virus 1
MLMICSAAEKGWVT

SEQ ID NO: 53 - protein, 12 aa, Human immunodeficiency virus 1
MIRSAAEKLWVT

SEQ ID NO: 54 - protein, 6 aa, Homo sapiens
EKGWVT

SEQ ID NO: 55 - protein, 6 aa, Human immunodeficiency virus 1
EKGWVT

SEQ ID NO: 56 - protein, 12 aa, Human immunodeficiency virus 1
MIRSAEEKLWVT

SEQ ID NO: 57 - protein, 10 aa, Homo sapiens
KIDRLLARLI

SEQ ID NO: 58 - protein, 10 aa, Human immunodeficiency virus 1
KIDRLLDRLI

SEQ ID NO: 59 - protein, 10 aa, Human immunodeficiency virus 1
KVDRLIARLI

SEQ ID NO: 60 - protein, 8 aa, Homo sapiens
KIDRLLAR

SEQ ID NO: 61 - protein, 8 aa, Human immunodeficiency virus 1
KIDRLLAR

SEQ ID NO: 62 - protein, 8 aa, Homo sapiens
RAAREKGC

SEQ ID NO: 63 - protein, 8 aa, Human immunodeficiency virus 1
RAARKKGC

SEQ ID NO: 64 - protein, 7 aa, Homo sapiens
KAGFAIL

SEQ ID NO: 65 - protein, 7 aa, Human immunodeficiency virus 1
KAGFAIL

SEQ ID NO: 66 - protein, 7 aa, Homo sapiens
AGFAILV

SEQ ID NO: 67 - protein, 7 aa, Human immunodeficiency virus 1
AGFAILI

SEQ ID NO: 68 - protein, 6 aa, Homo sapiens
AGFAIL

SEQ ID NO: 69 - protein, 6 aa, Human immunodeficiency virus 1
AGFAIL

SEQ ID NO: 70 - protein, 9 aa, Homo sapiens
DELREEGVR

SEQ ID NO: 71 - protein, 9 aa, Human immunodeficiency virus 1
EELKEEAVR

SEQ ID NO: 72 - protein, 6 aa, Homo sapiens
DELREE

SEQ ID NO: 73 - protein, 6 aa, Human immunodeficiency virus 1
DELREQ

SEQ ID NO: 74 - protein, 40 aa, Artificial Sequence
  Description of Artificial Sequence Synthetic peptide
GGGGGGGGGGGGGGGGGGGGGGGGGGGGGGGGGGGGGGGG

SEQ ID NO: 75 - protein, 40 aa, Artificial Sequence
  Description of Artificial Sequence Synthetic peptide
SSSSSSSSSSSSSSSSSSSSSSSSSSSSSSSSSSSSSSSS

SEQ ID NO: 76 - protein, 40 aa, Artificial Sequence
  Description of Artificial Sequence Synthetic peptide
AAAAAAAAAAAAAAAAAAAAAAAAAAAAAAAAAAAAAAAA

SEQ ID NO: 77 - protein, 12 aa, Artificial Sequence
  Synthetic peptide
MLRAAREKGRVT

SEQ ID NO: 78 - protein, 12 aa, Artificial Sequence
  Synthetic peptide
MLRAAREEGRVT

SEQ ID NO: 79 - protein, 10 aa, Artificial Sequence
  Synthetic peptide
KIDRPLARLI

SEQ ID NO: 80 - protein, 10 aa, Artificial Sequence
  Synthetic peptide
KIDRPLSRLI

SEQ ID NO: 81 - protein, 13 aa, Artificial Sequence
  Synthetic peptide
NGKQKKAGVAILV

SEQ ID NO: 82 - protein, 9 aa, Artificial Sequence
  Synthetic peptide
RPNLHLIGV

SEQ ID NO: 83 - protein, 9 aa, Artificial Sequence
  Synthetic peptide
KVIYRFSAI

SEQ ID NO: 84 - protein, 9 aa, Artificial Sequence
  Synthetic peptide
KVTYRFNTI

SEQ ID NO: 85 - protein, 9 aa, Artificial Sequence
  Synthetic peptide
IVYLENPMV

SEQ ID NO: 86 - protein, 9 aa, Artificial Sequence
  Synthetic peptide
IVCLKNPIV

SEQ ID NO: 87 - protein, 9 aa, Artificial Sequence
  Synthetic peptide
TPRHVIVRF

SEQ ID NO: 88 - protein, 9 aa, Artificial Sequence
  Synthetic peptide
TPRHILVRF

SEQ ID NO: 89 - protein, 9 aa, Artificial Sequence
  Synthetic peptide
TPRHILVKF

SEQ ID NO: 90 - protein, 9 aa, Artificial Sequence
  Synthetic peptide
RARMAKSIL

SEQ ID NO: 91 - protein, 9 aa, Artificial Sequence
  Synthetic peptide
RACIAKSIL

SEQ ID NO: 92 - protein, 9 aa, Artificial Sequence
  Synthetic peptide
RAHIAKSTL

SEQ ID NO: 93 - protein, 9 aa, Artificial Sequence
  Synthetic peptide
ISFPAKLSF

SEQ ID NO: 94 - protein, 9 aa, Artificial Sequence
  Synthetic peptide
ISYPATLGF

SEQ ID NO: 95 - protein, 9 aa, Artificial Sequence
  Synthetic peptide
SSPATDQSW

SEQ ID NO: 96 - protein, 9 aa, Artificial Sequence
  Synthetic peptide
SSLATEQSW

SEQ ID NO: 97 - protein, 9 aa, Artificial Sequence
  Synthetic peptide
KATVTKTVW

SEQ ID NO: 98 - protein, 9 aa, Artificial Sequence
  Synthetic peptide
KATVTKTAC

SEQ ID NO: 99 - protein, 9 aa, Artificial Sequence
  Synthetic peptide
RANRQPTTW

SEQ ID NO: 100 - protein, 9 aa, Artificial Sequence
  Synthetic peptide
RVNRQPTEW

SEQ ID NO: 101 - protein, 9 aa, Artificial Sequence
  Synthetic peptide
RVNRQATEW

SEQ ID NO: 102 - protein, 15 aa, Artificial Sequence
  Synthetic peptide
MGKKQNRKTGNSKTQ

SEQ ID NO: 103 - protein, 15 aa, Artificial Sequence
  Synthetic peptide
QNRKTGNSKTQSASP

SEQ ID NO: 104 - protein, 15 aa, Artificial Sequence
  Synthetic peptide
TGNSKTQSASPPPKE

SEQ ID NO: 105 - protein, 15 aa, Artificial Sequence
  Synthetic peptide
KTQSASPPPKERSSS

SEQ ID NO: 106 - protein, 15 aa, Artificial Sequence
  Synthetic peptide
ASPPPKERSSSPATE

SEQ ID NO: 107 - protein, 15 aa, Artificial Sequence
  Synthetic peptide
PKERSSSPATEQSWM

SEQ ID NO: 108 - protein, 15 aa, Artificial Sequence
  Synthetic peptide
SSSPATEQSWMENDF

SEQ ID NO: 109 - protein, 15 aa, Artificial Sequence
  Synthetic peptide
ATEQSWMENDFDELR

SEQ ID NO: 110 - protein, 15 aa, Artificial Sequence
  Synthetic peptide
SWMENDFDELREEGF

SEQ ID NO: 111 - protein, 15 aa, Artificial Sequence
  Synthetic peptide
NDFDELREEGFRRSN

SEQ ID NO: 112 - protein, 15 aa, Artificial Sequence
  Synthetic peptide
ELREEGFRRSNYSEL

SEQ ID NO: 113 - protein, 15 aa, Artificial Sequence
  Synthetic peptide
EGFRRSNYSELREDI

SEQ ID NO: 114 - protein, 15 aa, Artificial Sequence
  Synthetic peptide
RSNYSELREDIQTKG

SEQ ID NO: 115 - protein, 15 aa, Artificial Sequence
  Synthetic peptide
SELREDIQTKGKEVE

SEQ ID NO: 116 - protein, 15 aa, Artificial Sequence
  Synthetic peptide
EDIQTKGKEVENFEK

SEQ ID NO: 117 - protein, 15 aa, Artificial Sequence
  Synthetic peptide
TKGKEVENFEKNLEE

SEQ ID NO: 118 - protein, 15 aa, Artificial Sequence
  Synthetic peptide
EVENFEKNLEECITR

SEQ ID NO: 119 - protein, 15 aa, Artificial Sequence
  Synthetic peptide
FEKNLEECITRITNT

SEQ ID NO: 120 - protein, 15 aa, Artificial Sequence
  Synthetic peptide
LEECITRITNTEKCL

SEQ ID NO: 121 - protein, 15 aa, Artificial Sequence
  Synthetic peptide
ITRITNTEKCLKELM

SEQ ID NO: 122 - protein, 15 aa, Artificial Sequence
  Synthetic peptide
TNTEKCLKELMELKT

SEQ ID NO: 123 - protein, 15 aa, Artificial Sequence
  Synthetic peptide
KCLKELMELKTKARE

SEQ ID NO: 124 - protein, 15 aa, Artificial Sequence
  Synthetic peptide
ELMELKTKARELREE

SEQ ID NO: 125 - protein, 15 aa, Artificial Sequence
  Synthetic peptide
LKTKARELREECRSL

SEQ ID NO: 126 - protein, 15 aa, Artificial Sequence
  Synthetic peptide
ARELREECRSLRSRC

SEQ ID NO: 127 - protein, 15 aa, Artificial Sequence
  Synthetic peptide
REECRSLRSRCDQLE

SEQ ID NO: 128 - protein, 15 aa, Artificial Sequence
  Synthetic peptide
RSLRSRCDQLEERVS

SEQ ID NO: 129 - protein, 15 aa, Artificial Sequence
  Synthetic peptide
SRCDQLEERVSAMED

SEQ ID NO: 130 - protein, 15 aa, Artificial Sequence
  Synthetic peptide
QLEERVSAMEDEMNE

SEQ ID NO: 131 - protein, 15 aa, Artificial Sequence
  Synthetic peptide
RVSAMEDEMNEMKRE

SEQ ID NO: 132 - protein, 15 aa, Artificial Sequence
  Synthetic peptide
MEDEMNEMKREGKFR

SEQ ID NO: 133 - protein, 15 aa, Artificial Sequence
  Synthetic peptide
MNEMKREGKFREKRI

SEQ ID NO: 134 - protein, 15 aa, Artificial Sequence
  Synthetic peptide
KREGKFREKRIKRNE

SEQ ID NO: 135 - protein, 15 aa, Artificial Sequence
  Synthetic peptide
KFREKRIKRNEQSLQ

SEQ ID NO: 136 - protein, 15 aa, Artificial Sequence
  Synthetic peptide
KRIKRNEQSLQEIWD

SEQ ID NO: 137 - protein, 15 aa, Artificial Sequence
  Synthetic peptide
RNEQSLQEIWDYVKR

SEQ ID NO: 138 - protein, 15 aa, Artificial Sequence
  Synthetic peptide
SLQEIWDYVKRPNLR

SEQ ID NO: 139 - protein, 15 aa, Artificial Sequence
  Synthetic peptide
IWDYVKRPNLRLIGV

SEQ ID NO: 140 - protein, 15 aa, Artificial Sequence
  Synthetic peptide
VKRPNLRLIGVPESD

SEQ ID NO: 141 - protein, 15 aa, Artificial Sequence
  Synthetic peptide
NLRLIGVPESDVENG

SEQ ID NO: 142 - protein, 15 aa, Artificial Sequence
  Synthetic peptide
IGVPESDVENGTKLE

SEQ ID NO: 143 - protein, 15 aa, Artificial Sequence
  Synthetic peptide
ESDVENGTKLENTLQ

SEQ ID NO: 144 - protein, 15 aa, Artificial Sequence
  Synthetic peptide
ENGTKLENTLQDIIQ

SEQ ID NO: 145 - protein, 15 aa, Artificial Sequence
  Synthetic peptide
KLENTLQDIIQENFP

SEQ ID NO: 146 - protein, 15 aa, Artificial Sequence
  Synthetic peptide
TLQDIIQENFPNLAR

SEQ ID NO: 147 - protein, 15 aa, Artificial Sequence
  Synthetic peptide
IIQENFPNLARQANV

SEQ ID NO: 148 - protein, 15 aa, Artificial Sequence
  Synthetic peptide
NFPNLARQANVQIQE

SEQ ID NO: 149 - protein, 15 aa, Artificial Sequence
  Synthetic peptide
LARQANVQIQEIQRT

SEQ ID NO: 150 - protein, 15 aa, Artificial Sequence
  Synthetic peptide
ANVQIQEIQRTPQRY

SEQ ID NO: 151 - protein, 15 aa, Artificial Sequence
  Synthetic peptide
IQEIQRTPQRYSSRR

SEQ ID NO: 152 - protein, 15 aa, Artificial Sequence
  Synthetic peptide
QRTPQRYSSRRATPR

SEQ ID NO: 153 - protein, 15 aa, Artificial Sequence
  Synthetic peptide
QRYSSRRATPRHIIV

SEQ ID NO: 154 - protein, 15 aa, Artificial Sequence
  Synthetic peptide
SRRATPRHIIVRFTK

SEQ ID NO: 155 - protein, 15 aa, Artificial Sequence
  Synthetic peptide
TPRHIIVRFTKVEMK

SEQ ID NO: 156 - protein, 15 aa, Artificial Sequence
  Synthetic peptide
IIVRFTKVEMKEKML

SEQ ID NO: 157 - protein, 15 aa, Artificial Sequence
  Synthetic peptide
FTKVEMKEKMLRAAR

SEQ ID NO: 158 - protein, 15 aa, Artificial Sequence
  Synthetic peptide
EMKEKMLRAAREKGR

SEQ ID NO: 159 - protein, 15 aa, Artificial Sequence
  Synthetic peptide
KMLRAAREKGRVTLK

SEQ ID NO: 160 - protein, 15 aa, Artificial Sequence
  Synthetic peptide
AAREKGRVTLKGKPI

SEQ ID NO: 161 - protein, 15 aa, Artificial Sequence
  Synthetic peptide
KGRVTLKGKPIRLTA

SEQ ID NO: 162 - protein, 15 aa, Artificial Sequence
  Synthetic peptide
TLKGKPIRLTADLSA

SEQ ID NO: 163 - protein, 15 aa, Artificial Sequence
  Synthetic peptide
KPIRLTADLSAETLQ

SEQ ID NO: 164 - protein, 15 aa, Artificial Sequence
  Synthetic peptide
LTADLSAETLQARRE

SEQ ID NO: 165 - protein, 15 aa, Artificial Sequence
  Synthetic peptide
LSAETLQARREWGPI

SEQ ID NO: 166 - protein, 15 aa, Artificial Sequence
  Synthetic peptide
TLQARREWGPIFNIL

SEQ ID NO: 167 - protein, 15 aa, Artificial Sequence
  Synthetic peptide
RREWGPIFNILKEKN

SEQ ID NO: 168 - protein, 15 aa, Artificial Sequence
  Synthetic peptide
GPIFNILKEKNFQPR

SEQ ID NO: 169 - protein, 15 aa, Artificial Sequence
  Synthetic peptide
NILKEKNFQPRISYP

SEQ ID NO: 170 - protein, 15 aa, Artificial Sequence
  Synthetic peptide
EKNFQPRISYPAKLS

SEQ ID NO: 171 - protein, 15 aa, Artificial Sequence
  Synthetic peptide
QPRISYPAKLSFISE

SEQ ID NO: 172 - protein, 15 aa, Artificial Sequence
  Synthetic peptide
SYPAKLSFISEGEIK

SEQ ID NO: 173 - protein, 15 aa, Artificial Sequence
  Synthetic peptide
KLSFISEGEIKYFID

SEQ ID NO: 174 - protein, 15 aa, Artificial Sequence
  Synthetic peptide
ISEGEIKYFIDKQML

SEQ ID NO: 175 - protein, 15 aa, Artificial Sequence
  Synthetic peptide
EIKYFIDKQMLRDFV

SEQ ID NO: 176 - protein, 15 aa, Artificial Sequence
  Synthetic peptide
FIDKQMLRDFVTTRP

SEQ ID NO: 177 - protein, 15 aa, Artificial Sequence
  Synthetic peptide
QMLRDFVTTRPALKE

SEQ ID NO: 178 - protein, 15 aa, Artificial Sequence
  Synthetic peptide
DFVTTRPALKELLKE

SEQ ID NO: 179 - protein, 15 aa, Artificial Sequence
  Synthetic peptide
TRPALKELLKEALNM

SEQ ID NO: 180 - protein, 15 aa, Artificial Sequence
  Synthetic peptide
LKELLKEALNMERNN

SEQ ID NO: 181 - protein, 15 aa, Artificial Sequence
  Synthetic peptide
LKEALNMERNNRYQP

SEQ ID NO: 182 - protein, 15 aa, Artificial Sequence
  Synthetic peptide
LNMERNNRYQPLQNH

SEQ ID NO: 183 - protein, 13 aa, Artificial Sequence
  Synthetic peptide
RNNRYQPLQNHAK

SEQ ID NO: 184 - protein, 15 aa, Artificial Sequence
  Synthetic peptide
MTGSNSHITILTLNI

SEQ ID NO: 185 - protein, 15 aa, Artificial Sequence
  Synthetic peptide
NSHITILTLNINGLN

SEQ ID NO: 186 - protein, 15 aa, Artificial Sequence
  Synthetic peptide
TILTLNINGLNSAIK

SEQ ID NO: 187 - protein, 15 aa, Artificial Sequence
  Synthetic peptide
LNINGLNSAIKRHRL

SEQ ID NO: 188 - protein, 15 aa, Artificial Sequence
  Synthetic peptide
GLNSAIKRHRLASWI

SEQ ID NO: 189 - protein, 15 aa, Artificial Sequence
  Synthetic peptide
AIKRHRLASWIKSQD

SEQ ID NO: 190 - protein, 15 aa, Artificial Sequence
  Synthetic peptide
HRLASWIKSQDPSVC

SEQ ID NO: 191 - protein, 15 aa, Artificial Sequence
  Synthetic peptide
SWIKSQDPSVCCIQE

SEQ ID NO: 192 - protein, 15 aa, Artificial Sequence
  Synthetic peptide
SQDPSVCCIQETHLT

SEQ ID NO: 193 - protein, 15 aa, Artificial Sequence
  Synthetic peptide
SVCCIQETHLTCRDT

SEQ ID NO: 194 - protein, 15 aa, Artificial Sequence
  Synthetic peptide
IQETHLTCRDTHRLK

SEQ ID NO: 195 - protein, 15 aa, Artificial Sequence
  Synthetic peptide
HLTCRDTHRLKIKGW

SEQ ID NO: 196 - protein, 15 aa, Artificial Sequence
  Synthetic peptide
RDTHRLKIKGWRKIY

SEQ ID NO: 197 - protein, 15 aa, Artificial Sequence
  Synthetic peptide
RLKIKGWRKIYQANG

SEQ ID NO: 198 - protein, 15 aa, Artificial Sequence
  Synthetic peptide
KGWRKIYQANGKQKK

SEQ ID NO: 199 - protein, 15 aa, Artificial Sequence
  Synthetic peptide
KIYQANGKQKKAGVA

SEQ ID NO: 200 - protein, 15 aa, Artificial Sequence
  Synthetic peptide
ANGKQKKAGVAILVS

SEQ ID NO: 201 - protein, 15 aa, Artificial Sequence
  Synthetic peptide
QKKAGVAILVSDKTD

SEQ ID NO: 202 - protein, 15 aa, Artificial Sequence
  Synthetic peptide
GVAILVSDKTDFKPT

SEQ ID NO: 203 - protein, 15 aa, Artificial Sequence
  Synthetic peptide
LVSDKTDFKPTKIKR

SEQ ID NO: 204 - protein, 15 aa, Artificial Sequence
  Synthetic peptide
KTDFKPTKIKRDKEG

SEQ ID NO: 205 - protein, 15 aa, Artificial Sequence
  Synthetic peptide
KPTKIKRDKEGHYIM

SEQ ID NO: 206 - protein, 15 aa, Artificial Sequence
  Synthetic peptide
IKRDKEGHYIMVKGS

SEQ ID NO: 207 - protein, 15 aa, Artificial Sequence
  Synthetic peptide
KEGHYIMVKGSIQQE

SEQ ID NO: 208 - protein, 15 aa, Artificial Sequence
  Synthetic peptide
YIMVKGSIQQEELTI

SEQ ID NO: 209 - protein, 15 aa, Artificial Sequence
  Synthetic peptide
KGSIQQEELTILNIY

SEQ ID NO: 210 - protein, 15 aa, Artificial Sequence
  Synthetic peptide
QQEELTILNIYAPNT

SEQ ID NO: 211 - protein, 15 aa, Artificial Sequence
  Synthetic peptide
LTILNIYAPNTGAPR

SEQ ID NO: 212 - protein, 15 aa, Artificial Sequence
  Synthetic peptide
NIYAPNTGAPRFIKQ

SEQ ID NO: 213 - protein, 15 aa, Artificial Sequence
  Synthetic peptide
PNTGAPRFIKQVLSD

SEQ ID NO: 214 - protein, 15 aa, Artificial Sequence
  Synthetic peptide
APRFIKQVLSDLQRD

SEQ ID NO: 215 - protein, 15 aa, Artificial Sequence
  Synthetic peptide
IKQVLSDLQRDLDSH

SEQ ID NO: 216 - protein, 15 aa, Artificial Sequence
  Synthetic peptide
LSDLQRDLDSHTLIM

SEQ ID NO: 217 - protein, 15 aa, Artificial Sequence
  Synthetic peptide
QRDLDSHTLIMGDFN

SEQ ID NO: 218 - protein, 15 aa, Artificial Sequence
  Synthetic peptide
DSHTLIMGDFNTPLS

SEQ ID NO: 219 - protein, 15 aa, Artificial Sequence
  Synthetic peptide
LIMGDFNTPLSTLDR

SEQ ID NO: 220 - protein, 15 aa, Artificial Sequence
  Synthetic peptide
DFNTPLSTLDRSTRQ

SEQ ID NO: 221 - protein, 15 aa, Artificial Sequence
  Synthetic peptide
PLSTLDRSTRQKVNK

SEQ ID NO: 222 - protein, 15 aa, Artificial Sequence
  Synthetic peptide
LDRSTRQKVNKDTQE

SEQ ID NO: 223 - protein, 15 aa, Artificial Sequence
  Synthetic peptide
TRQKVNKDTQELNSA

SEQ ID NO: 224 - protein, 15 aa, Artificial Sequence
  Synthetic peptide
VNKDTQELNSALHQA

SEQ ID NO: 225 - protein, 15 aa, Artificial Sequence
  Synthetic peptide
TQELNSALHQADLID

SEQ ID NO: 226 - protein, 15 aa, Artificial Sequence
  Synthetic peptide
NSALHQADLIDIYRT

SEQ ID NO: 227 - protein, 15 aa, Artificial Sequence
  Synthetic peptide
HQADLIDIYRTLHPK

SEQ ID NO: 228 - protein, 15 aa, Artificial Sequence
  Synthetic peptide
LIDIYRTLHPKSTEY

SEQ ID NO: 229 - protein, 15 aa, Artificial Sequence
  Synthetic peptide
YRTLHPKSTEYTFFS

SEQ ID NO: 230 - protein, 15 aa, Artificial Sequence
  Synthetic peptide
HPKSTEYTFFSAPHH

SEQ ID NO: 231 - protein, 15 aa, Artificial Sequence
  Synthetic peptide
TEYTFFSAPHHTYSK

SEQ ID NO: 232 - protein, 15 aa, Artificial Sequence
  Synthetic peptide
FFSAPHHTYSKIDHI

SEQ ID NO: 233 - protein, 15 aa, Artificial Sequence
  Synthetic peptide
PHHTYSKIDHIVGSK

SEQ ID NO: 234 - protein, 15 aa, Artificial Sequence
  Synthetic peptide
YSKIDHIVGSKALLS

SEQ ID NO: 235 - protein, 15 aa, Artificial Sequence
  Synthetic peptide
DHIVGSKALLSKCKR

SEQ ID NO: 236 - protein, 15 aa, Artificial Sequence
  Synthetic peptide
GSKALLSKCKRTEII

SEQ ID NO: 237 - protein, 15 aa, Artificial Sequence
  Synthetic peptide
LLSKCKRTEIITNYL

SEQ ID NO: 238 - protein, 15 aa, Artificial Sequence
  Synthetic peptide
CKRTEIITNYLSDHS

SEQ ID NO: 239 - protein, 15 aa, Artificial Sequence
  Synthetic peptide
EIITNYLSDHSAIKL

SEQ ID NO: 240 - protein, 15 aa, Artificial Sequence
  Synthetic peptide
NYLSDHSAIKLELRI

SEQ ID NO: 241 - protein, 15 aa, Artificial Sequence
  Synthetic peptide
DHSAIKLELRIKNLT

SEQ ID NO: 242 - protein, 15 aa, Artificial Sequence
  Synthetic peptide
IKLELRIKNLTQSRS

SEQ ID NO: 243 - protein, 15 aa, Artificial Sequence
  Synthetic peptide
LRIKNLTQSRSTTWK

SEQ ID NO: 244 - protein, 15 aa, Artificial Sequence
  Synthetic peptide
NLTQSRSTTWKLNNL

SEQ ID NO: 245 - protein, 15 aa, Artificial Sequence
  Synthetic peptide
SRSTTWKLNNLLLND

SEQ ID NO: 246 - protein, 15 aa, Artificial Sequence
  Synthetic peptide
TWKLNNLLLNDYWVH

SEQ ID NO: 247 - protein, 15 aa, Artificial Sequence
  Synthetic peptide
NNLLLNDYWVHNEMK

SEQ ID NO: 248 - protein, 15 aa, Artificial Sequence
  Synthetic peptide
LNDYWVHNEMKAEIK

SEQ ID NO: 249 - protein, 15 aa, Artificial Sequence
  Synthetic peptide
WVHNEMKAEIKMFFE

SEQ ID NO: 250 - protein, 15 aa, Artificial Sequence
  Synthetic peptide
EMKAEIKMFFETNEN

SEQ ID NO: 251 - protein, 15 aa, Artificial Sequence
  Synthetic peptide
EIKMFFETNENKDTT

SEQ ID NO: 252 - protein, 15 aa, Artificial Sequence
  Synthetic peptide
FFETNENKDTTYQNL

SEQ ID NO: 253 - protein, 15 aa, Artificial Sequence
  Synthetic peptide
NENKDTTYQNLWDAF

SEQ ID NO: 254 - protein, 15 aa, Artificial Sequence
  Synthetic peptide
DTTYQNLWDAFKAVC

SEQ ID NO: 255 - protein, 15 aa, Artificial Sequence
  Synthetic peptide
QNLWDAFKAVCRGKF

SEQ ID NO: 256 - protein, 15 aa, Artificial Sequence
  Synthetic peptide
DAFKAVCRGKFIALN

SEQ ID NO: 257 - protein, 15 aa, Artificial Sequence
  Synthetic peptide
AVCRGKFIALNAYKR

SEQ ID NO: 258 - protein, 15 aa, Artificial Sequence
  Synthetic peptide
GKFIALNAYKRKQER

SEQ ID NO: 259 - protein, 15 aa, Artificial Sequence
  Synthetic peptide
ALNAYKRKQERSKID

SEQ ID NO: 260 - protein, 15 aa, Artificial Sequence
  Synthetic peptide
YKRKQERSKIDTLTS

SEQ ID NO: 261 - protein, 15 aa, Artificial Sequence
  Synthetic peptide
QERSKIDTLTSQLKE

SEQ ID NO: 262 - protein, 15 aa, Artificial Sequence
  Synthetic peptide
KIDTLTSQLKELEKQ

SEQ ID NO: 263 - protein, 15 aa, Artificial Sequence
  Synthetic peptide
LTSQLKELEKQEQTH

SEQ ID NO: 264 - protein, 15 aa, Artificial Sequence
  Synthetic peptide
LKELEKQEQTHSKAS

SEQ ID NO: 265 - protein, 15 aa, Artificial Sequence
  Synthetic peptide
EKQEQTHSKASRRQE

SEQ ID NO: 266 - protein, 15 aa, Artificial Sequence
  Synthetic peptide
QTHSKASRRQEITKI

SEQ ID NO: 267 - protein, 15 aa, Artificial Sequence
  Synthetic peptide
KASRRQEITKIRAEL

SEQ ID NO: 268 - protein, 15 aa, Artificial Sequence
  Synthetic peptide
RQEITKIRAELKEIE

SEQ ID NO: 269 - protein, 15 aa, Artificial Sequence
  Synthetic peptide
TKIRAELKEIETQKT

SEQ ID NO: 270 - protein, 15 aa, Artificial Sequence
  Synthetic peptide
AELKEIETQKTLQKI

SEQ ID NO: 271 - protein, 15 aa, Artificial Sequence
  Synthetic peptide
EIETQKTLQKINESR

SEQ ID NO: 272 - protein, 15 aa, Artificial Sequence
  Synthetic peptide
QKTLQKINESRSWFF

SEQ ID NO: 273 - protein, 15 aa, Artificial Sequence
  Synthetic peptide
QKINESRSWFFERIN

SEQ ID NO: 274 - protein, 15 aa, Artificial Sequence
  Synthetic peptide
ESRSWFFERINKIDR

SEQ ID NO: 275 - protein, 15 aa, Artificial Sequence
  Synthetic peptide
WFFERINKIDRPLAR

SEQ ID NO: 276 - protein, 15 aa, Artificial Sequence
  Synthetic peptide
RINKIDRPLARLIKK

SEQ ID NO: 277 - protein, 15 aa, Artificial Sequence
  Synthetic peptide
IDRPLARLIKKKREK

SEQ ID NO: 278 - protein, 15 aa, Artificial Sequence
  Synthetic peptide
LARLIKKKREKNQID

SEQ ID NO: 279 - protein, 15 aa, Artificial Sequence
  Synthetic peptide
IKKKREKNQIDTIKN

SEQ ID NO: 280 - protein, 15 aa, Artificial Sequence
  Synthetic peptide
REKNQIDTIKNDKGD

SEQ ID NO: 281 - protein, 15 aa, Artificial Sequence
  Synthetic peptide
QIDTIKNDKGDITTD

SEQ ID NO: 282 - protein, 15 aa, Artificial Sequence
  Synthetic peptide
IKNDKGDITTDPTEI

SEQ ID NO: 283 - protein, 15 aa, Artificial Sequence
  Synthetic peptide
KGDITTDPTEIQTTI

SEQ ID NO: 284 - protein, 15 aa, Artificial Sequence
  Synthetic peptide
TTDPTEIQTTIREYY

SEQ ID NO: 285 - protein, 15 aa, Artificial Sequence
  Synthetic peptide
TEIQTTIREYYKHLY

SEQ ID NO: 286 - protein, 15 aa, Artificial Sequence
  Synthetic peptide
TTIREYYKHLYANKL

SEQ ID NO: 287 - protein, 15 aa, Artificial Sequence
  Synthetic peptide
EYYKHLYANKLENLE

SEQ ID NO: 288 - protein, 15 aa, Artificial Sequence
  Synthetic peptide
HLYANKLENLEEMDT

SEQ ID NO: 289 - protein, 15 aa, Artificial Sequence
  Synthetic peptide
NKLENLEEMDTFLDT

SEQ ID NO: 290 - protein, 15 aa, Artificial Sequence
  Synthetic peptide
NLEEMDTFLDTYTLP

SEQ ID NO: 291 - protein, 15 aa, Artificial Sequence
  Synthetic peptide
MDTFLDTYTLPRLNQ

SEQ ID NO: 292 - protein, 15 aa, Artificial Sequence
  Synthetic peptide
LDTYTLPRLNQEEVE

SEQ ID NO: 293 - protein, 15 aa, Artificial Sequence
  Synthetic peptide
TLPRLNQEEVESLNR

SEQ ID NO: 294 - protein, 15 aa, Artificial Sequence
  Synthetic peptide
LNQEEVESLNRPITG

SEQ ID NO: 295 - protein, 15 aa, Artificial Sequence
  Synthetic peptide
EVESLNRPITGSEIV

SEQ ID NO: 296 - protein, 15 aa, Artificial Sequence
  Synthetic peptide
LNRPITGSEIVAIIN

SEQ ID NO: 297 - protein, 15 aa, Artificial Sequence
  Synthetic peptide
ITGSEIVAIINSLPT

SEQ ID NO: 298 - protein, 15 aa, Artificial Sequence
  Synthetic peptide
EIVAIINSLPTKKSP

SEQ ID NO: 299 - protein, 15 aa, Artificial Sequence
  Synthetic peptide
IINSLPTKKSPGPDG

SEQ ID NO: 300 - protein, 15 aa, Artificial Sequence
  Synthetic peptide
LPTKKSPGPDGFTAE

SEQ ID NO: 301 - protein, 15 aa, Artificial Sequence
  Synthetic peptide
KSPGPDGFTAEFYQR

SEQ ID NO: 302 - protein, 15 aa, Artificial Sequence
  Synthetic peptide
PDGFTAEFYQRYKEE

SEQ ID NO: 303 - protein, 15 aa, Artificial Sequence
  Synthetic peptide
TAEFYQRYKEELVPF

SEQ ID NO: 304 - protein, 15 aa, Artificial Sequence
  Synthetic peptide
YQRYKEELVPFLLKL

SEQ ID NO: 305 - protein, 15 aa, Artificial Sequence
  Synthetic peptide
KEELVPFLLKLFQSI

SEQ ID NO: 306 - protein, 15 aa, Artificial Sequence
  Synthetic peptide
VPFLLKLFQSIEKEG

SEQ ID NO: 307 - protein, 15 aa, Artificial Sequence
  Synthetic peptide
LKLFQSIEKEGILPN

SEQ ID NO: 308 - protein, 15 aa, Artificial Sequence
  Synthetic peptide
QSIEKEGILPNSFYE

SEQ ID NO: 309 - protein, 15 aa, Artificial Sequence
  Synthetic peptide
KEGILPNSFYEASII

SEQ ID NO: 310 - protein, 15 aa, Artificial Sequence
  Synthetic peptide
LPNSFYEASIILIPK

SEQ ID NO: 311 - protein, 15 aa, Artificial Sequence
  Synthetic peptide
FYEASIILIPKPGRD

SEQ ID NO: 312 - protein, 15 aa, Artificial Sequence
  Synthetic peptide
SIILIPKPGRDTTKK

SEQ ID NO: 313 - protein, 15 aa, Artificial Sequence
  Synthetic peptide
IPKPGRDTTKKENFR

SEQ ID NO: 314 - protein, 15 aa, Artificial Sequence
  Synthetic peptide
GRDTTKKENFRPISL

SEQ ID NO: 315 - protein, 15 aa, Artificial Sequence
  Synthetic peptide
TKKENFRPISLMNID

SEQ ID NO: 316 - protein, 15 aa, Artificial Sequence
  Synthetic peptide
NFRPISLMNIDAKIL

SEQ ID NO: 317 - protein, 15 aa, Artificial Sequence
  Synthetic peptide
ISLMNIDAKILNKIL

SEQ ID NO: 318 - protein, 15 aa, Artificial Sequence
  Synthetic peptide
NIDAKILNKILANRI

SEQ ID NO: 319 - protein, 15 aa, Artificial Sequence
  Synthetic peptide
KILNKILANRIQQHI

SEQ ID NO: 320 - protein, 15 aa, Artificial Sequence
  Synthetic peptide
KILANRIQQHIKKLI

SEQ ID NO: 321 - protein, 15 aa, Artificial Sequence
  Synthetic peptide
NRIQQHIKKLIHHDQ

SEQ ID NO: 322 - protein, 15 aa, Artificial Sequence
  Synthetic peptide
QHIKKLIHHDQVGFI

SEQ ID NO: 323 - protein, 15 aa, Artificial Sequence
  Synthetic peptide
KLIHHDQVGFIPGMQ

SEQ ID NO: 324 - protein, 15 aa, Artificial Sequence
  Synthetic peptide
HDQVGFIPGMQGWFN

SEQ ID NO: 325 - protein, 15 aa, Artificial Sequence
  Synthetic peptide
GFIPGMQGWFNIRKS

SEQ ID NO: 326 - protein, 15 aa, Artificial Sequence
  Synthetic peptide
GMQGWFNIRKSINVI

SEQ ID NO: 327 - protein, 15 aa, Artificial Sequence
  Synthetic peptide
WFNIRKSINVIQHIN

SEQ ID NO: 328 - protein, 15 aa, Artificial Sequence
  Synthetic peptide
RKSINVIQHINRAKD

SEQ ID NO: 329 - protein, 15 aa, Artificial Sequence
  Synthetic peptide
NVIQHINRAKDKNHM

SEQ ID NO: 330 - protein, 15 aa, Artificial Sequence
  Synthetic peptide
HINRAKDKNHMIISI

SEQ ID NO: 331 - protein, 15 aa, Artificial Sequence
  Synthetic peptide
AKDKNHMIISIDAEK

SEQ ID NO: 332 - protein, 15 aa, Artificial Sequence
  Synthetic peptide
NHMIISIDAEKAFDK

SEQ ID NO: 333 - protein, 15 aa, Artificial Sequence
  Synthetic peptide
ISIDAEKAFDKIQQP

SEQ ID NO: 334 - protein, 15 aa, Artificial Sequence
  Synthetic peptide
AEKAFDKIQQPFMLK

SEQ ID NO: 335 - protein, 15 aa, Artificial Sequence
  Synthetic peptide
FDKIQQPFMLKTLNK

SEQ ID NO: 336 - protein, 15 aa, Artificial Sequence
  Synthetic peptide
QQPFMLKTLNKLGID

SEQ ID NO: 337 - protein, 15 aa, Artificial Sequence
  Synthetic peptide
MLKTLNKLGIDGTYF

SEQ ID NO: 338 - protein, 15 aa, Artificial Sequence
  Synthetic peptide
LNKLGIDGTYFKIIR

SEQ ID NO: 339 - protein, 15 aa, Artificial Sequence
  Synthetic peptide
GIDGTYFKIIRAIYD

SEQ ID NO: 340 - protein, 15 aa, Artificial Sequence
  Synthetic peptide
TYFKIIRAIYDKPTA

SEQ ID NO: 341 - protein, 15 aa, Artificial Sequence
  Synthetic peptide
IIRAIYDKPTANIIL

SEQ ID NO: 342 - protein, 15 aa, Artificial Sequence
  Synthetic peptide
IYDKPTANIILNGQK

SEQ ID NO: 343 - protein, 15 aa, Artificial Sequence
  Synthetic peptide
PTANIILNGQKLEAF

SEQ ID NO: 344 - protein, 15 aa, Artificial Sequence
  Synthetic peptide
IILNGQKLEAFPLKT

SEQ ID NO: 345 - protein, 15 aa, Artificial Sequence
  Synthetic peptide
GQKLEAFPLKTGTRQ

SEQ ID NO: 346 - protein, 15 aa, Artificial Sequence
  Synthetic peptide
EAFPLKTGTRQGCPL

SEQ ID NO: 347 - protein, 15 aa, Artificial Sequence
  Synthetic peptide
LKTGTRQGCPLSPLL

SEQ ID NO: 348 - protein, 15 aa, Artificial Sequence
  Synthetic peptide
TRQGCPLSPLLFNIV

SEQ ID NO: 349 - protein, 15 aa, Artificial Sequence
  Synthetic peptide
CPLSPLLFNIVLEVL

SEQ ID NO: 350 - protein, 15 aa, Artificial Sequence
  Synthetic peptide
PLLFNIVLEVLARAI

SEQ ID NO: 351 - protein, 15 aa, Artificial Sequence
  Synthetic peptide
NIVLEVLARAIRQEK

SEQ ID NO: 352 - protein, 15 aa, Artificial Sequence
  Synthetic peptide
EVLARAIRQEKEIKG

SEQ ID NO: 353 - protein, 15 aa, Artificial Sequence
  Synthetic peptide
RAIRQEKEIKGIQLG

SEQ ID NO: 354 - protein, 15 aa, Artificial Sequence
  Synthetic peptide
QEKEIKGIQLGKEEV

SEQ ID NO: 355 - protein, 15 aa, Artificial Sequence
  Synthetic peptide
IKGIQLGKEEVKLSL

SEQ ID NO: 356 - protein, 15 aa, Artificial Sequence
  Synthetic peptide
QLGKEEVKLSLFADD

SEQ ID NO: 357 - protein, 15 aa, Artificial Sequence
  Synthetic peptide
EEVKLSLFADDMIVY

SEQ ID NO: 358 - protein, 15 aa, Artificial Sequence
  Synthetic peptide
LSLFADDMIVYLENP

SEQ ID NO: 359 - protein, 15 aa, Artificial Sequence
  Synthetic peptide
ADDMIVYLENPIVSA

SEQ ID NO: 360 - protein, 15 aa, Artificial Sequence
  Synthetic peptide
IVYLENPIVSAQNLL

SEQ ID NO: 361 - protein, 15 aa, Artificial Sequence
  Synthetic peptide
ENPIVSAQNLLKLIS

SEQ ID NO: 362 - protein, 15 aa, Artificial Sequence
  Synthetic peptide
VSAQNLLKLISNFSK

SEQ ID NO: 363 - protein, 15 aa, Artificial Sequence
  Synthetic peptide
NLLKLISNFSKVSGY

SEQ ID NO: 364 - protein, 15 aa, Artificial Sequence
  Synthetic peptide
LISNFSKVSGYKINV

SEQ ID NO: 365 - protein, 15 aa, Artificial Sequence
  Synthetic peptide
FSKVSGYKINVQKSQ

SEQ ID NO: 366 - protein, 15 aa, Artificial Sequence
  Synthetic peptide
SGYKINVQKSQAFLY

SEQ ID NO: 367 - protein, 15 aa, Artificial Sequence
  Synthetic peptide
INVQKSQAFLYTNNR

SEQ ID NO: 368 - protein, 15 aa, Artificial Sequence
  Synthetic peptide
KSQAFLYTNNRQTES

SEQ ID NO: 369 - protein, 15 aa, Artificial Sequence
  Synthetic peptide
FLYTNNRQTESQIMG

SEQ ID NO: 370 - protein, 15 aa, Artificial Sequence
  Synthetic peptide
NNRQTESQIMGELPF

SEQ ID NO: 371 - protein, 15 aa, Artificial Sequence
  Synthetic peptide
TESQIMGELPFTIAS

SEQ ID NO: 372 - protein, 15 aa, Artificial Sequence
  Synthetic peptide
IMGELPFTIASKRIK

SEQ ID NO: 373 - protein, 15 aa, Artificial Sequence
  Synthetic peptide
LPFTIASKRIKYLGI

SEQ ID NO: 374 - protein, 15 aa, Artificial Sequence
  Synthetic peptide
IASKRIKYLGIQLTR

SEQ ID NO: 375 - protein, 15 aa, Artificial Sequence
  Synthetic peptide
RIKYLGIQLTRDVKD

SEQ ID NO: 376 - protein, 15 aa, Artificial Sequence
  Synthetic peptide
LGIQLTRDVKDLFKE

SEQ ID NO: 377 - protein, 15 aa, Artificial Sequence
  Synthetic peptide
LTRDVKDLFKENYKP

SEQ ID NO: 378 - protein, 15 aa, Artificial Sequence
  Synthetic peptide
VKDLFKENYKPLLKE

SEQ ID NO: 379 - protein, 15 aa, Artificial Sequence
  Synthetic peptide
FKENYKPLLKEIKEE

SEQ ID NO: 380 - protein, 15 aa, Artificial Sequence
  Synthetic peptide
YKPLLKEIKEETNKW

SEQ ID NO: 381 - protein, 15 aa, Artificial Sequence
  Synthetic peptide
LKEIKEETNKWKNIP

SEQ ID NO: 382 - protein, 15 aa, Artificial Sequence
  Synthetic peptide
KEETNKWKNIPCSWV

SEQ ID NO: 383 - protein, 15 aa, Artificial Sequence
  Synthetic peptide
NKWKNIPCSWVGRIN

SEQ ID NO: 384 - protein, 15 aa, Artificial Sequence
  Synthetic peptide
NIPCSWVGRINIVKM

SEQ ID NO: 385 - protein, 15 aa, Artificial Sequence
  Synthetic peptide
SWVGRINIVKMAILP

SEQ ID NO: 386 - protein, 15 aa, Artificial Sequence
  Synthetic peptide
RINIVKMAILPKVIY

SEQ ID NO: 387 - protein, 15 aa, Artificial Sequence
  Synthetic peptide
VKMAILPKVIYRFNA

SEQ ID NO: 388 - protein, 15 aa, Artificial Sequence
  Synthetic peptide
ILPKVIYRFNAIPIK

SEQ ID NO: 389 - protein, 15 aa, Artificial Sequence
  Synthetic peptide
VIYRFNAIPIKLPMT

SEQ ID NO: 390 - protein, 15 aa, Artificial Sequence
  Synthetic peptide
FNAIPIKLPMTFFTE

SEQ ID NO: 391 - protein, 15 aa, Artificial Sequence
  Synthetic peptide
PIKLPMTFFTELEKT

SEQ ID NO: 392 - protein, 15 aa, Artificial Sequence
  Synthetic peptide
PMTFFTELEKTTLKF

SEQ ID NO: 393 - protein, 15 aa, Artificial Sequence
  Synthetic peptide
FTELEKTTLKFIWNQ

SEQ ID NO: 394 - protein, 15 aa, Artificial Sequence
  Synthetic peptide
EKTTLKFIWNQKRAR

SEQ ID NO: 395 - protein, 15 aa, Artificial Sequence
  Synthetic peptide
LKFIWNQKRARIAKS

SEQ ID NO: 396 - protein, 15 aa, Artificial Sequence
  Synthetic peptide
WNQKRARIAKSILSQ

SEQ ID NO: 397 - protein, 15 aa, Artificial Sequence
  Synthetic peptide
RARIAKSILSQKNKA

SEQ ID NO: 398 - protein, 15 aa, Artificial Sequence
  Synthetic peptide
AKSILSQKNKAGGIT

SEQ ID NO: 399 - protein, 15 aa, Artificial Sequence
  Synthetic peptide
LSQKNKAGGITLPDF

SEQ ID NO: 400 - protein, 15 aa, Artificial Sequence
  Synthetic peptide
NKAGGITLPDFKLYY

SEQ ID NO: 401 - protein, 15 aa, Artificial Sequence
  Synthetic peptide
GITLPDFKLYYKATV

SEQ ID NO: 402 - protein, 15 aa, Artificial Sequence
  Synthetic peptide
PDFKLYYKATVTKTA

SEQ ID NO: 403 - protein, 15 aa, Artificial Sequence
  Synthetic peptide
LYYKATVTKTAWYWY

SEQ ID NO: 404 - protein, 15 aa, Artificial Sequence
  Synthetic peptide
ATVTKTAWYWYQNRD

SEQ ID NO: 405 - protein, 15 aa, Artificial Sequence
  Synthetic peptide
KTAWYWYQNRDIDQW

SEQ ID NO: 406 - protein, 15 aa, Artificial Sequence
  Synthetic peptide
YWYQNRDIDQWNRTE

SEQ ID NO: 407 - protein, 15 aa, Artificial Sequence
  Synthetic peptide
NRDIDQWNRTEPSEI

SEQ ID NO: 408 - protein, 15 aa, Artificial Sequence
  Synthetic peptide
DQWNRTEPSEIMPHI

SEQ ID NO: 409 - protein, 15 aa, Artificial Sequence
  Synthetic peptide
RTEPSEIMPHIYNYL

SEQ ID NO: 410 - protein, 15 aa, Artificial Sequence
  Synthetic peptide
SEIMPHIYNYLIFDK

SEQ ID NO: 411 - protein, 15 aa, Artificial Sequence
  Synthetic peptide
PHIYNYLIFDKPEKN

SEQ ID NO: 412 - protein, 15 aa, Artificial Sequence
  Synthetic peptide
NYLIFDKPEKNKQWG

SEQ ID NO: 413 - protein, 15 aa, Artificial Sequence
  Synthetic peptide
FDKPEKNKQWGKDSL

SEQ ID NO: 414 - protein, 15 aa, Artificial Sequence
  Synthetic peptide
EKNKQWGKDSLFNKW

SEQ ID NO: 415 - protein, 15 aa, Artificial Sequence
  Synthetic peptide
QWGKDSLFNKWCWEN

SEQ ID NO: 416 - protein, 15 aa, Artificial Sequence
  Synthetic peptide
DSLFNKWCWENWLAI

SEQ ID NO: 417 - protein, 15 aa, Artificial Sequence
  Synthetic peptide
NKWCWENWLAICRKL

SEQ ID NO: 418 - protein, 15 aa, Artificial Sequence
  Synthetic peptide
WENWLAICRKLKLDP

SEQ ID NO: 419 - protein, 15 aa, Artificial Sequence
  Synthetic peptide
LAICRKLKLDPFLTP

SEQ ID NO: 420 - protein, 15 aa, Artificial Sequence
  Synthetic peptide
RKLKLDPFLTPYTKI

SEQ ID NO: 421 - protein, 15 aa, Artificial Sequence
  Synthetic peptide
LDPFLTPYTKINSRW

SEQ ID NO: 422 - protein, 15 aa, Artificial Sequence
  Synthetic peptide
LTPYTKINSRWIKDL

SEQ ID NO: 423 - protein, 15 aa, Artificial Sequence
  Synthetic peptide
TKINSRWIKDLNVKP

SEQ ID NO: 424 - protein, 15 aa, Artificial Sequence
  Synthetic peptide
SRWIKDLNVKPKTIK

SEQ ID NO: 425 - protein, 15 aa, Artificial Sequence
  Synthetic peptide
KDLNVKPKTIKTLEE

SEQ ID NO: 426 - protein, 15 aa, Artificial Sequence
  Synthetic peptide
VKPKTIKTLEENLGI

SEQ ID NO: 427 - protein, 15 aa, Artificial Sequence
  Synthetic peptide
TIKTLEENLGITIQD

SEQ ID NO: 428 - protein, 15 aa, Artificial Sequence
  Synthetic peptide
LEENLGITIQDIGVG

SEQ ID NO: 429 - protein, 15 aa, Artificial Sequence
  Synthetic peptide
LGITIQDIGVGKDFM

SEQ ID NO: 430 - protein, 15 aa, Artificial Sequence
  Synthetic peptide
IQDIGVGKDFMSKTP

SEQ ID NO: 431 - protein, 15 aa, Artificial Sequence
  Synthetic peptide
GVGKDFMSKTPKAMA

SEQ ID NO: 432 - protein, 15 aa, Artificial Sequence
  Synthetic peptide
DFMSKTPKAMATKDK

SEQ ID NO: 433 - protein, 15 aa, Artificial Sequence
  Synthetic peptide
KTPKAMATKDKIDKW

SEQ ID NO: 434 - protein, 15 aa, Artificial Sequence
  Synthetic peptide
AMATKDKIDKWDLIK

SEQ ID NO: 435 - protein, 15 aa, Artificial Sequence
  Synthetic peptide
KDKIDKWDLIKLKSF

SEQ ID NO: 436 - protein, 15 aa, Artificial Sequence
  Synthetic peptide
DKWDLIKLKSFCTAK

SEQ ID NO: 437 - protein, 15 aa, Artificial Sequence
  Synthetic peptide
LIKLKSFCTAKETTI

SEQ ID NO: 438 - protein, 15 aa, Artificial Sequence
  Synthetic peptide
KSFCTAKETTIRVNR

SEQ ID NO: 439 - protein, 15 aa, Artificial Sequence
  Synthetic peptide
TAKETTIRVNRQPTT

SEQ ID NO: 440 - protein, 15 aa, Artificial Sequence
  Synthetic peptide
TTIRVNRQPTTWEKI

SEQ ID NO: 441 - protein, 15 aa, Artificial Sequence
  Synthetic peptide
VNRQPTTWEKIFATY

SEQ ID NO: 442 - protein, 15 aa, Artificial Sequence
  Synthetic peptide
PTTWEKIFATYSSDK

SEQ ID NO: 443 - protein, 15 aa, Artificial Sequence
  Synthetic peptide
EKIFATYSSDKGLIS

SEQ ID NO: 444 - protein, 15 aa, Artificial Sequence
  Synthetic peptide
ATYSSDKGLISRIYN

SEQ ID NO: 445 - protein, 15 aa, Artificial Sequence
  Synthetic peptide
SDKGLISRIYNELKQ

SEQ ID NO: 446 - protein, 15 aa, Artificial Sequence
  Synthetic peptide
LISRIYNELKQIYKK

SEQ ID NO: 447 - protein, 15 aa, Artificial Sequence
  Synthetic peptide
IYNELKQIYKKKTNN

SEQ ID NO: 448 - protein, 15 aa, Artificial Sequence
  Synthetic peptide
LKQIYKKKTNNPIKK

SEQ ID NO: 449 - protein, 15 aa, Artificial Sequence
  Synthetic peptide
YKKKTNNPIKKWAKD

SEQ ID NO: 450 - protein, 15 aa, Artificial Sequence
  Synthetic peptide
TNNPIKKWAKDMNRH

SEQ ID NO: 451 - protein, 15 aa, Artificial Sequence
  Synthetic peptide
IKKWAKDMNRHFSKE

SEQ ID NO: 452 - protein, 15 aa, Artificial Sequence
  Synthetic peptide
AKDMNRHFSKEDIYA

SEQ ID NO: 453 - protein, 15 aa, Artificial Sequence
  Synthetic peptide
NRHFSKEDIYAAKKH

SEQ ID NO: 454 - protein, 15 aa, Artificial Sequence
  Synthetic peptide
SKEDIYAAKKHMKKC

SEQ ID NO: 455 - protein, 15 aa, Artificial Sequence
  Synthetic peptide
IYAAKKHMKKCSSSL

SEQ ID NO: 456 - protein, 15 aa, Artificial Sequence
  Synthetic peptide
KKHMKKCSSSLAIRE

SEQ ID NO: 457 - protein, 15 aa, Artificial Sequence
  Synthetic peptide
KKCSSSLAIREMQIK

SEQ ID NO: 458 - protein, 15 aa, Artificial Sequence
  Synthetic peptide
SSLAIREMQIKTTMR

SEQ ID NO: 459 - protein, 15 aa, Artificial Sequence
  Synthetic peptide
IREMQIKTTMRYHLT

SEQ ID NO: 460 - protein, 15 aa, Artificial Sequence
  Synthetic peptide
QIKTTMRYHLTPVRM

SEQ ID NO: 461 - protein, 15 aa, Artificial Sequence
  Synthetic peptide
TMRYHLTPVRMAIIK

SEQ ID NO: 462 - protein, 15 aa, Artificial Sequence
  Synthetic peptide
HLTPVRMAIIKKSGN

SEQ ID NO: 463 - protein, 15 aa, Artificial Sequence
  Synthetic peptide
VRMAIIKKSGNNRCW

SEQ ID NO: 464 - protein, 15 aa, Artificial Sequence
  Synthetic peptide
IIKKSGNNRCWRGCG

SEQ ID NO: 465 - protein, 15 aa, Artificial Sequence
  Synthetic peptide
SGNNRCWRGCGEIGT

SEQ ID NO: 466 - protein, 15 aa, Artificial Sequence
  Synthetic peptide
RCWRGCGEIGTLLHC

SEQ ID NO: 467 - protein, 15 aa, Artificial Sequence
  Synthetic peptide
GCGEIGTLLHCWWDC

SEQ ID NO: 468 - protein, 15 aa, Artificial Sequence
  Synthetic peptide
IGTLLHCWWDCKLVQ

SEQ ID NO: 469 - protein, 15 aa, Artificial Sequence
  Synthetic peptide
LHCWWDCKLVQPLWK

SEQ ID NO: 470 - protein, 15 aa, Artificial Sequence
  Synthetic peptide
WDCKLVQPLWKSVWR

SEQ ID NO: 471 - protein, 15 aa, Artificial Sequence
  Synthetic peptide
LVQPLWKSVWRFLRD

SEQ ID NO: 472 - protein, 15 aa, Artificial Sequence
  Synthetic peptide
LWKSVWRFLRDLELE

SEQ ID NO: 473 - protein, 15 aa, Artificial Sequence
  Synthetic peptide
VWRFLRDLELEIPFD

SEQ ID NO: 474 - protein, 15 aa, Artificial Sequence
  Synthetic peptide
LRDLELEIPFDPAIP

SEQ ID NO: 475 - protein, 15 aa, Artificial Sequence
  Synthetic peptide
ELEIPFDPAIPLLGI

SEQ ID NO: 476 - protein, 15 aa, Artificial Sequence
  Synthetic peptide
PFDPAIPLLGIYPNE

SEQ ID NO: 477 - protein, 15 aa, Artificial Sequence
  Synthetic peptide
AIPLLGIYPNEYKSC

SEQ ID NO: 478 - protein, 15 aa, Artificial Sequence
  Synthetic peptide
LGIYPNEYKSCCYKD

SEQ ID NO: 479 - protein, 15 aa, Artificial Sequence
  Synthetic peptide
PNEYKSCCYKDTCTR

SEQ ID NO: 480 - protein, 15 aa, Artificial Sequence
  Synthetic peptide
KSCCYKDTCTRMFIA

SEQ ID NO: 481 - protein, 15 aa, Artificial Sequence
  Synthetic peptide
YKDTCTRMFIAALFT

SEQ ID NO: 482 - protein, 15 aa, Artificial Sequence
  Synthetic peptide
CTRMFIAALFTIAKT

SEQ ID NO: 483 - protein, 15 aa, Artificial Sequence
  Synthetic peptide
FIAALFTIAKTWNQP

SEQ ID NO: 484 - protein, 15 aa, Artificial Sequence
  Synthetic peptide
LFTIAKTWNQPKCPT

SEQ ID NO: 485 - protein, 15 aa, Artificial Sequence
  Synthetic peptide
AKTWNQPKCPTMIDW

SEQ ID NO: 486 - protein, 15 aa, Artificial Sequence
  Synthetic peptide
NQPKCPTMIDWIKKM

SEQ ID NO: 487 - protein, 15 aa, Artificial Sequence
  Synthetic peptide
CPTMIDWIKKMWHIY

SEQ ID NO: 488 - protein, 15 aa, Artificial Sequence
  Synthetic peptide
IDWIKKMWHIYTMEY

SEQ ID NO: 489 - protein, 15 aa, Artificial Sequence
  Synthetic peptide
KKMWHIYTMEYYAAI

SEQ ID NO: 490 - protein, 15 aa, Artificial Sequence
  Synthetic peptide
HIYTMEYYAAIKNDE

SEQ ID NO: 491 - protein, 15 aa, Artificial Sequence
  Synthetic peptide
MEYYAAIKNDEFISF

SEQ ID NO: 492 - protein, 15 aa, Artificial Sequence
  Synthetic peptide
AAIKNDEFISFVGTW

SEQ ID NO: 493 - protein, 15 aa, Artificial Sequence
  Synthetic peptide
NDEFISFVGTWMKLE

SEQ ID NO: 494 - protein, 15 aa, Artificial Sequence
  Synthetic peptide
ISFVGTWMKLETIIL

SEQ ID NO: 495 - protein, 15 aa, Artificial Sequence
  Synthetic peptide
GTWMKLETIILSKLS

SEQ ID NO: 496 - protein, 15 aa, Artificial Sequence
  Synthetic peptide
KLETIILSKLSQEQK

SEQ ID NO: 497 - protein, 15 aa, Artificial Sequence
  Synthetic peptide
IILSKLSQEQKTKHR

SEQ ID NO: 498 - protein, 15 aa, Artificial Sequence
  Synthetic peptide
KLSQEQKTKHRIFSL

SEQ ID NO: 499 - protein, 15 aa, Artificial Sequence
  Synthetic peptide
EQKTKHRIFSLIGGN